(19) United States
(12) Patent Application Publication
(10) Pub. No.: US 2009/0169550 A1
(43) Pub. Date: Jul. 2, 2009

(54) THERAPY OF RITUXIMAB-REFRACTORY RHEUMATOID ARTHRITIS PATIENTS

(75) Inventor: WOLFGANG DUMMER, San Francisco, CA (US)

Correspondence Address: GENENTECH, INC., 1 DNA WAY, SOUTH SAN FRANCISCO, CA, 94080, US

(73) Assignee: Genentech, Inc., South San Francisco, CA (US)

(21) Appl. No.: 12/339,476

(22) Filed: Dec. 19, 2008

Related U.S. Application Data
(60) Provisional application No. 61/016,281, filed on Dec. 21, 2007.

(51) Int. Cl. A61K 39/395

(52) U.S. Cl. 424/133.1; 424/144.1

(57) ABSTRACT

A method is disclosed of treating a rituximab-refractory rheumatoid arthritis (RA) patient comprising administering an anti-CD20 antibody other than rituximab to the patient in an amount effective to treat the RA.

### CROSS REFERENCE TO RELATED APPLICATION

This non-provisional application filed under 37 CFR § 1.53(b), claims the benefit under 35 USC § 119(e) of U.S. Provisional Application Ser. No. 61/016,281 filed on 21 Dec. 2007, which is incorporated by reference in entirety.

### FIELD OF THE INVENTION

The present invention concerns methods for treating rituximab-refractory rheumatoid arthritis (RA) patients.

### BACKGROUND OF THE INVENTION

Lymphocytes are one of many types of white blood cells produced in the bone marrow during the process of hematopoiesis. There are two major populations of lymphocytes: B-lymphocytes (B cells) and T-lymphocytes (T cells).

B cells mature within the bone marrow and leave the marrow expressing an antigen-binding antibody on their cell surface. When a naïve B cell first encounters the antigen for which its membrane-bound antibody is specific, the cell begins to divide rapidly and its progeny differentiate into memory B cells and effector cells called “plasma cells.” Memory B cells have a longer life span and continue to express membrane-bound antibody with the same specificity as the original parent cell. Plasma cells do not produce membrane-bound antibody, but instead produce the antibody in a form that can be secreted. Secreted antibodies are the major effector molecules of humoral immunity.

B-cell-related disorders include autoimmune diseases. Physicians and scientists have identified more than 70 clinically distinct autoimmune diseases, including RA, multiple sclerosis (MS), vasculitis, immune-mediated diabetes, and lupus such as systemic lupus erythematosus (SLE). The chronic nature of these diseases leads to an immense social and financial burden.

Cytotoxic agents that target B-cell surface antigens are an important focus of B-cell-related cancer therapies. Such B-cell surface antigens, including CD19, CD20, CD22, and CD52, represent targets of therapeutic potential for treatment of lymphoma.

The CD20 antigen (also called human B-lymphocyte-restricted differentiation antigen, Bp35, or B1) is a four-pass, glycosylated integral membrane protein with a molecular weight of approximately 35 kD located on pre-B and mature B lymphocytes. The antigen is also expressed on greater than 90% of B-cell non-Hodgkin's lymphomas (NHL), but is not found on hematopoietic stem cells, pro-B cells, normal plasma cells, or other normal tissues. CD20 regulates early step(s) in the activation process for cell-cycle initiation and differentiation, and possibly functions as a calcium-ion channel. Undergoing phosphorylation in activated B cells, CD20 appears on the surface of B-lymphocytes at the pre-B-cell stage and is found on mature and memory B cells, but not plasma cells. CD20 has calcium-channel activity and may have a role in the development of B cells. The relationship between lysis of peripheral CD20⁺ B cells in vitro and rituximab activity in vivo is unclear. Rituximab displays antibody-dependent cellular cytotoxicity (ADCC) in vitro. Potent complement-dependent cytotoxic (CDC) activity has also been observed for rituximab on lymphoma cells and cell lines and in certain mouse xenograft models. Several anti-CD20 antibodies, including rituximab, have been shown to induce apoptosis in vitro when crosslinked by a secondary antibody or by other means.

Given the expression of CD20 in B-cell lymphomas, this antigen can serve as a candidate for “targeting” of such lymphomas. In essence, such targeting can be generalized as follows: antibodies specific to the CD20 surface antigen of B cells are administered to a patient. These anti-CD20 antibodies specifically bind to the CD20 antigen of (ostensibly) both normal and malignant B cells; the antibody bound to the CD20 surface antigen may lead to the destruction and depletion of neoplastic B cells. Additionally, chemical agents or radioactive labels having the potential to destroy the tumor can be conjugated to the anti-CD20 antibody such that the agent is specifically “delivered” to the neoplastic B cells. Irrespective of the approach, a primary goal is to destroy the tumor; the specific approach can be determined by the particular anti-CD20 antibody that is utilized, and thus, the available approaches to targeting the CD20 antigen can vary considerably.

The rituximab (RITUXAN®) antibody is a genetically engineered chimeric murine/human monoclonal antibody directed against the CD20 antigen. Rituximab is the antibody called “C2B8” in U.S. Pat. No. 5,736,137 (Anderson et al.). Rituximab is indicated for the treatment of patients with relapsed or refractory low-grade or follicular, CD20-positive, B-cell NHL. In vitro mechanism-of-action studies have demonstrated that rituximab binds human complement and lyses lymphoid B-cell lines through CDC. Additionally, it has significant activity in assays for ADCC. Rituximab has been shown to have anti-proliferative effects in tritiated thymidine-incorporation assays and to induce apoptosis directly, while other anti-CD19 and anti-CD20 antibodies do not. Rituximab sensitizes drug-resistant human B-cell lymphoma cell lines to the cytotoxic effects of doxorubicin and other toxins. In vivo preclinical studies have shown that rituximab depletes B cells from the peripheral blood, lymph nodes, and bone marrow of cynomolgus monkeys.

Rituximab was approved in the U.S. in November 1997 for the treatment of patients with relapsed or refractory low-grade or follicular CD20⁺ B-cell NHL at a dose of 375 mg/m² weekly for four doses. In April 2001, rituximab was additionally approved in the U.S. for treating low-grade NHL: re-treatment (weekly for four doses) and an additional dosing regimen (weekly for eight doses). Since approval, patients have been exposed to rituximab either as monotherapy or in combination with immunosuppressant or chemotherapeutic drugs. Patients have also been treated with rituximab as maintenance therapy for up to two years. Rituximab has been used in the treatment of malignant and nonmalignant plasma cell disorders.

Rituximab has also been studied in a variety of non-malignant autoimmune disorders. Such disorders include RA, lupus, immune thrombocytopenic purpura, pure red cell aplasia, autoimmune anemia, cold agglutinin disease, type B syndrome of severe insulin resistance, mixed cryoglobulinemia, myasthenia gravis, Wegener's granulomatosis, refractory pemphigus vulgaris, dermatomyositis, Sjögren's syndrome, active type-II mixed cryoglobulinemia, pemphigus vulgaris, autoimmune neuropathy, paraneoplastic opsoclonus-myoclonus syndrome, and relapsing-remitting multiple sclerosis (RRMS).

A Phase II study (WA16291) was conducted in patients with RA, providing 48-week follow-up data on safety and efficacy of rituximab. Patients were evenly randomized to four treatment arms: methotrexate, rituximab alone, rituximab plus methotrexate, and rituximab plus cyclophosphamide. The treatment regimen of rituximab was one gram administered intravenously on days 1 and 15. Infusions of rituximab were well tolerated by most RA patients, 36% of whom experienced at least one adverse event during their first infusion (compared with 30% of patients receiving placebo). Overall, the majority of adverse events was considered to be mild to moderate in severity and was well balanced across all treatment groups. Nineteen total serious adverse events occurred across the four arms over the 48 weeks, which were slightly more frequent in the rituximab/cyclophosphamide group. The incidence of infections was well balanced across all groups. The mean rate of serious infection in this RA patient population was 4.66 per 100 patient-years, which is lower than the rate of infections requiring hospital admission in RA patients (9.57 per 100 patient-years) reported in a community-based epidemiologic study.

The DANCER Phase IIb trial evaluated the efficacy of rituximab and methotrexate in disease-modifying anti-rheumatic drug (DMARD)-resistant RA patients, with rituximab given at doses of 500 mg or 1000 mg at days 1 and 15. The ACR responses for both doses of rituximab were statistically superior to placebo at 6 months. No difference between the two rituximab doses was seen, and analysis of the utility of the oral corticosteroids revealed no significant impact on ACR response.

The REFLEX Phase 3 trial evaluated the efficacy of rituximab and methotrexate in RA patients with an inadequate response to anti-TNF-alpha therapy, with rituximab given at a dose of 1000 mg. Patients treated with rituximab under the trial conditions had demonstrated improvements in the signs and symptoms of active disease, with a significant benefit over six months.

The reported safety profile of rituximab in a small number of patients with neurologic disorders, including autoimmune neuropathy, opsoclonus-myoclonus syndrome, and RRMS, was similar to that reported in oncology or RA. In an ongoing investigator-sponsored trial (IST) of rituximab in combination with interferon-beta or glatiramer acetate in patients with RRMS, one of ten treated patients was admitted to the hospital for overnight observation after experiencing moderate fever and rigors following the first infusion of rituximab, while the other nine patients completed the four-infusion regimen without any reported adverse events.

Patients with refractory ANCA-associated vasculitis (AAV) were given rituximab along with immunosuppressive medicaments such as intravenous cyclophosphamide, mycophenolate mofetil, azathioprine, or leflunomide, with apparent efficacy. Rituximab was given in four doses along with intravenous cyclophosphamide at 375 mg/m² per dose to patients with refractory systemic vasculitis. Nine patients with AAV were successfully treated with two or four weekly doses of 500 mg of rituximab. In 11 patients with refractory AAV, rituximab treatment or re-treatment with four weekly doses at 375 mg/m²/dose induced remission by B-cell depletion.

Other anti-CD20 antibodies include, e.g, the ⁹⁰Y-labeled 2B8 murine antibody designated “Y2B8” (ZEVALIN®) (Biogen-Idec, Inc.) (e.g., U.S. Pat. No. 5,736,137, Anderson et al.; ATCC deposit HB11388); murine IgG2a “B1” or “tositumomab,” optionally labeled with ¹³¹I to produce the “131I-B1” or “iodine I131 tositumomab” antibody (BEXXAR™) (Corixa; Coulter Pharmaceutical, Inc.) (e.g., U.S. Pat. No. 5,595,721, Kaminski et al.); murine monoclonal antibody “1F5” (e.g., Press et al. Blood, 69(2):584-591 (1987) and its variants, e.g., “framework patched” or humanized 1F5 (e.g., WO 2003/002607, Leung; ATCC deposit HB-96450); murine and chimeric 2H7 antibody (e.g., U.S. Pat. No. 5,677,180, Robinson et al.); humanized 2H7 antibodies such as rhuMAb2H7 and other versions (Genentech, Inc.) (e.g., WO 2004/056312, Adams et al., and other references noted below); the human antibody targeted at CD20 called 2F2, HUMAX-CD20™, or ofatumumab (GlaxoSmithKline; GenMab A/S) (e.g., Glennie and van de Winkel, Drug Discovery Today, 8:503-510 (2003); Cragg et al., Blood, 101: 1045-1052 (2003); and US 2004/0167319, Teeling et al.); human monoclonal antibodies against CD20 (GenMab A/S/Medarex, Inc.) (e.g., WO 2004/035607 and WO 2005/103081, Teeling et al.); antibodies to CD20 having complex N-glycoside-linked sugar chains bound to the Fc region (Kyowa Hakko) (e.g., US 2004/0093621, Shitara et al.); a chimerized or humanized monoclonal antibody binding to an extracellular epitope of CD20 (Biomedics Inc.) (e.g., WO 2006/106959, Numazaki et al.); monoclonal antibodies and fragments binding to CD20 (e.g., WO 2005/000901, Tedder et al.) such as HB20-3, HB20-4, HB20-25, and MB20-11; small, modular immunopharmaceuticals (SMIPs) binding to CD20 (Wyeth, Trubion Pharmaceuticals, Inc.), including TRU-015 (e.g., US 2005/0186216; US 2005/0202534; US 2005/0202028; US 2005/136049; and US 2005/0202023, Ledbetter et al., and US 2007/0059306, Grosmaire et al.); CD20-binding antibodies including the AME series of antibodies (Eli Lilly and Co., Applied Molecular Evolution, Inc.), such as AME 33 (e.g., US 2005/0025764, Watkins et al.) and AME 133 and AME 133v antibodies (e.g., US 2005/0136044, Watkins and Pancook) (see also, e.g., WO 2004/103404 and US 2006/0251652, Watkins et al.) and the anti-CD20 antibodies with Fc mutations (e.g., WO 2005/070963, Allan et al.); CD20-binding molecules such as those set forth in WO 2005/016969 and US 2005/0069545, Carr et al.); bispecific antibodies set forth in WO 2005/014618 (Chang et al.); humanized LL2 and similar antibodies (Immunomedics, Inc.) (e.g., U.S. Pat. No. 7,151,164 and US 2005/0106108, Hansen); A20 antibodies (Immunomedics, Inc.) such as chimeric A20 (cA20) or humanized A20 antibody (hA20, IMMUN-106T, veltuzumab) (e.g., US 2003/0219433, Hansen et al.); fully human antibodies against CD20 (Amgen/AstraZeneca) (e.g., WO 2006/130458, Gazit et al.); antibodies against CD20 (Avestha Gengraine Technologies Pvt Ltd.) (e.g., WO 2006/126069, Morawala); chimeric or humanized B-Ly1 antibodies to CD20 (Roche/GlycArt Biotechnology AG) such as GA101 (e.g., WO 2005/044859; US 2005/0123546; US 2004/0072290; and US 2003/0175884, Umana et al.); and monoclonal antibodies L27, G28-2, 93-1B3, B-C1, or NU-B2 available from the International Leukocyte Typing Workshop (e.g., Valentine et al., In: Leukocyte Typing III (McMichael, Ed., p. 440, Oxford University Press (1987)). This list provides representative anti-CD20 antibodies, but is not exhaustive.

Despite rituximab being effective in treating B-cell lymphomas, it has been reported that about 50% of patients with relapsed/refractory CD20⁺ follicular lymphomas do not respond to initial therapy with rituximab (innate resistance) (McLaughlin et al., J Clin Oncol., 16:2825-2833 (1998)); and close to 60% of prior rituximab-responding patients will no longer benefit from retreatment with rituximab (acquired resistance) (Davis et al., J Clin Oncol., 18:3135-3143 (2000)). Whether these forms of rituximab resistance are due to an adaptive property of the malignant B cell or to an impaired host's immune effector mechanisms is unclear.

Bello and Sotomayor, Hematology, 1:233-242 (2007) discuss and review the literature regarding tumor- and host-related mechanisms to explain rituximab resistance. In suggesting therapeutic strategies to overcome these barriers to anti-CD20 therapy, they address a newer generation of monoclonal antibodies, including AME 133v, GA101, ofatumumab, and veltuzumab, the development of which has been spurred by the widespread use of anti-CD20 monoclonal antibody therapy.

Other literature describing lymphomas refractory to rituximab (e.g., B-cell lymphomas and indolent NHL) include US 2005/0025764 (Watkins et al.); US 2005/0202023 (Ledbetter et al.); US 2006/0246004 (Lowman); U.S. Pat. No. 6,455,043; US2003/0026804; US2003/0206903 (last three Grillo-Lopez); US 2004/0192900 (Kunz et al.); WO 2006/094192 (Goldenberg et al.); US 2007/0059306 and WO 2007/14278 (Grosmaire et al.); and WO 2007/14238 (Bruge). Fleischmann R. et al. “Safety and Efficacy in Rheumatoid Arthritis (RA) Patients (Pts) Treated With Rituximab who develop Human Anti-Chimeric Antibodies (HACA)” (ACR Late Breaker Abstract 2007) reports a small number of RA patients developing high-titer anti-rituximab antibodies (HACA) that appeared to interfere with B-cell depletion.

A need exists for an effective means to treat RA and joint damage in those patients resistant, refractory, or otherwise not responsive to treatment with rituximab.

### SUMMARY OF THE INVENTION

Accordingly, the present invention provides a means for treating patients with RA and joint damage who are not responsive to rituximab. The invention is as claimed.

In one aspect, the invention provides a method of treating a rheumatoid arthritis (RA) patient who is not responsive to rituximab comprising administering an anti-CD20 antibody to the patient in an amount effective to treat the RA, wherein the anti-CD20 antibody is (1) ofatumumab comprising the variable light amino acid sequence in SEQ ID NO:2 and the variable heavy amino acid sequence in SEQ ID NO:4 or in SEQ ID NO:5; (2) veltuzumab comprising the variable light amino acid sequence in SEQ ID NO:7 and the variable heavy amino acid sequence in SEQ ID NO:8 or in SEQ ID NO:9; (3) an immunopharmaceutical comprising SEQ ID NO: 11; (4) a CD20-binding antibody comprising the variable light amino acid sequence in SEQ ID NO: 13 and the variable heavy amino acid sequence in SEQ ID NO: 15, or comprising the variable light amino acid sequence in SEQ ID NO: 17 and the variable heavy amino acid sequence in SEQ ID NO: 18, or comprising SEQ ID NO: 19; or (5) a humanized type II anti-CD20 IgG1 antibody with bisected afucosylated carbohydrates in its Fc region and comprising the variable light amino acid sequence in SEQ ID NO:21 and the variable heavy amino acid sequence in SEQ ID NO:23.

In another aspect, the invention provides a method of treating a rheumatoid arthritis (RA) patient who is not responsive to rituximab comprising administering an anti-CD20 antibody to the patient in an amount effective to treat the RA, wherein the anti-CD20 antibody is (1) ofatumumab having the light- and heavy-chain variable regions of SEQ ID NOS:2 and 4, respectively, or of SEQ ID NOS:2 and 5, respectively; (2) veltuzumab having the light- and heavy-chain variable regions of SEQ ID NOS:7 and 8, respectively, or of SEQ ID NOS:7 and 9, respectively; (3) an immunopharmaceutical having SEQ ID NO:11; (4) a CD20-binding antibody having the light- and heavy-chain variable regions of SEQ ID NOS:13 and 15, respectively, or having the light- and heavy-chain variable regions of SEQ ID NOS:17 and 18, respectively, or having SEQ ID NO:19; or (5) a humanized type II anti-CD20 IgG1 antibody with bisected afucosylated carbohydrates in its Fc region and having the light- and heavy-chain variable regions of SEQ ID NOS:21 and 23, respectively.

In a further aspect, the invention involves the use of an anti-CD20 antibody that is (1) ofatumumab comprising the variable light amino acid sequence in SEQ ID NO:2 and the variable heavy amino acid sequence in SEQ ID NO:4 or in SEQ ID NO:5; (2) veltuzumab comprising the variable light amino acid sequence in SEQ ID NO:7 and the variable heavy amino acid sequence in SEQ ID NO:8 or in SEQ ID NO:9; (3) an immunopharmaceutical comprising SEQ ID NO:11; (4) a CD20-binding antibody comprising the variable light amino acid sequence in SEQ ID NO:13 and the variable heavy amino acid sequence in SEQ ID NO:15, or comprising the variable light amino acid sequence in SEQ ID NO:17 and the variable heavy amino acid sequence in SEQ ID NO:18, or comprising SEQ ID NO:19; or (5) a humanized type II anti-CD20 IgG1 antibody with bisected afucosylated carbohydrates in its Fc region and comprising the variable light amino acid sequence in SEQ ID NO:21 and the variable heavy amino acid sequence in SEQ ID NO:23, in the manufacture of a pharmaceutical composition for treating a rheumatoid arthritis patient who is not responsive to rituximab.

In one embodiment, the anti-CD20 antibody is ofatumumab. Most preferably, the ofatumumab comprises the variable heavy amino acid sequence in SEQ ID NO:4. Alternatively, it comprises the variable heavy amino acid sequence in SEQ ID NO:5.

In another embodiment, the anti-CD20 antibody is veltuzumab. In one particular aspect, the veltuzumab comprises the variable heavy amino acid sequence in SEQ ID NO:8. In another specific aspect, the veltuzumab comprises the variable heavy amino acid sequence in SEQ ID NO:9.

In a further embodiment, the anti-CD20 antibody is the immunopharmaceutical (i.e., TRU-015).

In a still further embodiment, the anti-CD20 antibody is the CD20-binding antibody comprising SEQ ID NOS:13 and 15 or SEQ ID NOS:17 and 18 or SEQ ID NO:19. In one aspect, the CD20-binding antibody comprises the variable light amino acid sequence in SEQ ID NO:13 and the variable heavy amino acid sequence in SEQ ID NO:15.

In another aspect, the CD20-binding antibody comprises the variable light amino acid sequence in SEQ ID NO:17 and the variable heavy amino acid sequence in SEQ ID NO:18. In a further aspect, the CD20-binding antibody comprises SEQ ID NO:19.

In a fifth overall embodiment, the anti-CD20 antibody is the humanized type II anti-CD20 IgG1 antibody.

In a preferred aspect, the anti-CD20 antibody is not conjugated with a cytotoxic agent.

In other preferred aspects, the anti-CD20 antibody is administered intravenously or subcutaneously. In a specific aspect, it is administered subcutaneously.

In another preferred embodiment, the effective amount of the anti-CD20 antibody results in a clinical improvement as determined by assessing the number of tender or swollen joints, conducting a global clinical assessment of the patient, assessing erythrocyte sedimentation rate, assessing the amount of C-reactive protein level, or using composite measures of disease activity.

The anti-CD20 antibody is administered in a weight-based or fixed dose, depending on clinical parameters, for example. Preferred is the fixed dose. In a preferred aspect of the fixed dosing, the anti-CD20 antibody is administered in a dose of between about 50 and 4000 mg, more preferably between about 75 and 3000 mg, more preferably between about 100 and 2000 mg, more preferably between about 100 and 1000 mg, still more preferably between about 150 and 1000 mg, still more preferably between about 200 and 1000 mg, and most preferably the dose is about 200, 300, 400, 500, 600, 700, 800, 900, 1000 mg, or 2000 mg.

In another preferred embodiment, the antibody is administered at a frequency of one to four doses within a period of about one month. In another preferred aspect, the antibody is administered in two to three doses. In still further aspects, the antibody is administered within a period of about 2 to 3 weeks.

In another aspect, the method further comprises administering an effective amount of one or more second medicaments with the antibody, wherein the antibody is a first medicament. The second medicament is one medicament or more than one medicament. The second medicament is preferably an immunosuppressive agent, a DMARD, a different antibody against CD20 than the first medicament, a pain-control agent, an integrin antagonist, a non-steroidal anti-inflammatory drug (NSAID), a cytokine antagonist, a bisphosphonate, or a combination thereof.

In one aspect, the second medicament is a DMARD, preferably selected from the group consisting of auranofin, chloroquine, D-penicillamine, injectable gold, oral gold, hydroxychloroquine, sulfasalazine, myocrisin and methotrexate (MTX).

In another aspect, the second medicament is a NSAID, preferably selected from the group consisting of fenbufen, naprosyn, diclofenac, etodolac, indomethacin, aspirin, and ibuprofen.

In a further aspect, the second medicament is an immunosuppressive agent selected from the group consisting of etanercept, infliximab, adalimumab, leflunomide, anakinra, azathioprine, and cyclophosphamide.

In a still further aspect, the second medicament is selected from the group consisting of anti-a4, etanercept, infliximab, etanercept, adalimumab, kinaret, efalizumab, osteoprotegerin (OPG), anti-receptor activator of NFκB ligand (anti-RANKL), anti-receptor activator of NFκB-Fc (RANK-Fc), pamidronate, alendronate, actonel, zolendronate, rituximab, a 2H7 antibody, clodronate, MTX, azulfidine, hydroxychloroquine, doxycycline, leflunomide, sulfasalazine (SSZ), prednisolone, interleukin-1 receptor antagonist, prednisone, and methylprednisolone.

In yet another aspect, the second medicament is selected from the group consisting of infliximab, MTX, a combination of infliximab with MTX, etanercept, a corticosteroid, cyclosporin A, azathioprine, auranofin, hydroxychloroquine (HCQ), a combination of prednisolone with MTX and SSZ, a combination of MTX with SSZ and HCQ, a combination of cyclophosphamide with azathioprine and HCQ, and a combination of adalimumab with MTX. More preferably, the corticosteroid is prednisone, prednisolone, methylprednisolone, hydrocortisone, or dexamethasone.

The most preferred second medicament is MTX. The MTX is preferably administered perorally or parenterally.

In another preferred aspect of the method herein, the patient has exhibited an inadequate response to one or more anti-tumor necrosis factor-alpha inhibitors. In such a method the anti-CD20 antibody is preferably administered as a single dose or as two doses, with more preferably each dose being between about 200 mg and 1000 mg. Still more preferably, the dose is about 200 mg×2, about 300 mg×2, about 500 mg×2, about 700 mg×2, or about 1000 mg×2 on days 1 and 15 at the start of the treatment, preferably intravenously or subcutaneously.

In another preferred embodiment, the RA is early RA or incipient RA.

In an additional aspect, the method further comprises re-treating the patient by administering an effective amount of the anti-CD20 antibody to the patient. Preferably, the re-treatment is commenced at least about 24 weeks after the first administration of the antibody. In another embodiment, a further re-treatment is commenced, preferably at at least about 24 weeks after the second administration of the anti-CD20 antibody.

Preferably, joint damage has been reduced after the re-treatment and/or clinical improvement is observed in the patient before re-treatment. Preferably, such clinical improvement is determined by assessing the number of tender or swollen joints, conducting a global clinical assessment of the patient, assessing erythrocyte sedimentation rate, assessing the amount of C-reactive protein level, or using composite measures of disease activity.

In another aspect, the invention provides a method for treating joint damage in a subject who is not responsive to rituximab comprising administering to the subject an anti-CD20 antibody that is (1) ofatumumab comprising the variable light amino acid sequence in SEQ ID NO:2 and the variable heavy amino acid sequence in SEQ ID NO:4 or in SEQ ID NO:5; (2) veltuzumab comprising the variable light amino acid sequence in SEQ ID NO:7 and the variable heavy amino acid sequence in SEQ ID NO:8 or in SEQ ID NO:9; (3) an immunopharmaceutical comprising SEQ ID NO:11; (4) a CD20-binding antibody comprising the variable light amino acid sequence in SEQ ID NO:13 and the variable heavy amino acid sequence in SEQ ID NO:15, or comprising the variable light amino acid sequence in SEQ ID NO:17 and the variable heavy amino acid sequence in SEQ ID NO:18, or comprising SEQ ID NO:19; or (5) a humanized type II anti-CD20 IgG1 antibody with bisected afucosylated carbohydrates in its Fc region and comprising the variable light amino acid sequence in SEQ ID NO:21 and the variable heavy amino acid sequence in SEQ ID NO:23, wherein the amount of anti-CD20 antibody administered is effective in achieving a reduction in the joint damage.

In another aspect, the invention involves the use of an anti-CD20 antibody that is (1) ofatumumab comprising the variable light amino acid sequence in SEQ ID NO:2 and the variable heavy amino acid sequence in SEQ ID NO:4 or in SEQ ID NO:5; (2) veltuzumab comprising the variable light amino acid sequence in SEQ ID NO:7 and the variable heavy amino acid sequence in SEQ ID NO:8 or in SEQ ID NO:9; (3) an immunopharmaceutical comprising SEQ ID NO:11; (4) a CD20-binding antibody comprising the variable light amino acid sequence in SEQ ID NO:13 and the variable heavy amino acid sequence in SEQ ID NO:15, or comprising the variable light amino acid sequence in SEQ ID NO:17 and the variable heavy amino acid sequence in SEQ ID NO:18, or comprising SEQ ID NO:19; or (5) a humanized type II anti-CD20 IgG1 antibody with bisected afucosylated carbohydrates in its Fc region and comprising the variable light amino acid sequence in SEQ ID NO:21 and the variable heavy amino acid sequence in SEQ ID NO:23, in the manufacture of a pharmaceutical composition for treating joint damage in a subject who is not responsive to rituximab.

In this method, preferably radiographic testing is used to determine the extent of joint damage reduction. This test is preferably done at least about one month after administering the anti-CD20 antibody, more preferably at least about two months after administering the antibody.

In specific aspects, the anti-CD20 antibody is ofatumumab, or veltuzumab, or the immunopharmaceutical, or the CD20-binding antibody comprising SEQ ID NOS:13 and 15 or SEQ ID NOS:17 and 18 or SEQ ID NO:19, or the humanized type II anti-CD20 IgG1 antibody.

More preferably, the ofatumumab comprises the variable heavy amino acid sequence in SEQ ID NO:4. Alternatively, it comprises the variable heavy amino acid sequence in SEQ ID NO:5. In one particular aspect, the veltuzumab comprises the variable heavy amino acid sequence in SEQ ID NO:8. In another specific aspect, the veltuzumab comprises the variable heavy amino acid sequence in SEQ ID NO:9. In one aspect, the CD20-binding antibody comprises the variable light amino acid sequence in SEQ ID NO:13 and the variable heavy amino acid sequence in SEQ ID NO:15. In another aspect, the CD20-binding antibody comprises the variable light amino acid sequence in SEQ ID NO:17 and the variable heavy amino acid sequence in SEQ ID NO:18. In a further aspect, the CD20-binding antibody comprises SEQ ID NO:19.

Preferably, the joint damage treatment method further comprises administering an effective amount of one or more second medicaments with the anti-CD20 antibody, wherein the anti-CD20 antibody is a first medicament. The second medicament may be more than one medicament. The second medicament is preferably an immunosuppressive agent, a DMARD, a different antibody against CD20 than the first medicament, an integrin antagonist, a NSAID, a cytokine antagonist, a bisphosphonate, or a combination thereof.

In a still further embodiment, the invention provides a method for advertising an anti-CD20 antibody or a pharmaceutically acceptable composition thereof comprising promoting, to a target audience, the use of an anti-CD20 antibody that is (1) ofatumumab comprising the variable light amino acid sequence in SEQ ID NO:2 and the variable heavy amino acid sequence in SEQ ID NO:4 or in SEQ ID NO:5; (2) veltuzumab comprising the variable light amino acid sequence in SEQ ID NO:7 and the variable heavy amino acid sequence in SEQ ID NO:8 or in SEQ ID NO:9; (3) an immunopharmaceutical comprising SEQ ID NO:11; (4) a CD20-binding antibody comprising the variable light amino acid sequence in SEQ ID NO:13 and the variable heavy amino acid sequence in SEQ ID NO:15, or comprising the variable light amino acid sequence in SEQ ID NO:17 and the variable heavy amino acid sequence in SEQ ID NO:18, or comprising SEQ ID NO:19; or (5) a humanized type II anti-CD20 IgG1 antibody with bisected afucosylated carbohydrates in its Fc region and comprising the variable light amino acid sequence in SEQ ID NO:21 and the variable heavy amino acid sequence in SEQ ID NO:23, or a pharmaceutical composition thereof, for treating a rheumatoid arthritis patient who is not responsive to rituximab.

The invention herein also contemplates a method of monitoring the treatment of bone or soft tissue joint damage in a subject with RA or joint damage who is not responsive to rituximab comprising administering an effective amount of one of the anti-CD20 antibodies herein to the subject and measuring by imaging techniques such as MRI or radiography after at least about 3 months, preferably about 24 weeks, from the administration whether the bone or soft tissue joint damage has been reduced over baseline prior to the administration, wherein a decrease versus baseline in the subject after treatment indicates the anti-CD20 antibody is having an effect on the joint damage. Preferably, the degree of reduction versus baseline is measured a second time after the administration of the anti-CD20 antibody.

In yet another aspect, the invention provides a method of determining whether to continue administering an anti-CD20 antibody herein to a subject with bone or soft tissue joint damage who is not responsive to rituximab comprising measuring reduction in joint damage in the subject, using imaging techniques, such as radiography and/or MRI, after administration of the anti-CD20 antibody a first time, measuring reduction in joint damage in the subject, using imaging techniques such as radiography and/or MRI after administration of the anti-CD20 antibody a second time, comparing imaging findings in the subject at the first time and at the second time, and if the score is less at the second time than at the first time, continuing administration of the anti-CD20 antibody.

### DETAILED DESCRIPTION OF THE PREFERRED EMBODIMENTS

### I. Definitions

A “B cell” is a lymphocyte that matures within the bone marrow, and includes a naïve B cell, memory B cell, or effector B cell (plasma cell). The B cell herein is a normal or non-malignant B cell.

A “B-cell malignancy” is a malignancy involving B cells. Examples include Hodgkin's disease, including lymphocyte predominant Hodgkin's disease (LPHD); NHL; follicular center cell (FCC) lymphoma; acute lymphocytic leukemia (ALL); chronic lymphocytic leukemia (CLL); hairy cell leukemia; plasmacytoid lymphocytic lymphoma; mantle cell lymphoma; AIDS or HIV-related lymphoma; multiple myeloma; central nervous system (CNS) lymphoma; post-transplant lymphoproliferative disorder (PTLD); Waldenstrom's macroglobulinemia (lymphoplasmacytic lymphoma); mucosa-associated lymphoid tissue (MALT) lymphoma; and marginal zone lymphoma/leukemia.

The “CD20” antigen, or “CD20,” is an about 35-kDa, non-glycosylated phosphoprotein found on the surface of greater than 90% of B cells from peripheral blood or lymphoid organs. CD20 is present on both normal B cells as well as malignant B cells, but is not expressed on stem cells. Other names for CD20 in the literature include “B-lymphocyte-restricted antigen” and “Bp35.” The CD20 antigen is described in Clark et al., Proc. Natl. Acad. Sci. (USA), 82:1766 (1985), for example. The preferred CD20 antigen is human CD20.

The term “antibody” herein is used in the broadest sense and specifically covers monoclonal antibodies, polyclonal antibodies, multispecific antibodies (e.g. bispecific antibodies) formed from at least two intact antibodies, and antibody fragments, so long as they exhibit the desired biological activity.

An “isolated” antibody is one that has been identified and separated and/or recovered from a component of its natural environment. Contaminant components of its natural environment are materials that would interfere with research, diagnostic or therapeutic uses for the antibody, and may include enzymes, hormones, and other proteinaceous or nonproteinaceous solutes. In some embodiments, an antibody is purified (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of, for example, a spinning cup sequenator, or (3) to homogeneity by SDS-PAGE under reducing or nonreducing conditions using, for example, Coomassie blue or silver stain. Isolated antibody includes the antibody in situ within recombinant cells since at least one component of the antibody's natural environment will not be present. Ordinarily, however, isolated antibody will be prepared by at least one purification step.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light-chain variable domain is aligned with the variable domain of the heavy chain. Particular amino acid residues are believed to form an interface between the light-chain and heavy-chain variable domains.

The term “anti-CD20 antibodies” as used herein refers to (1) ofatumumab ((HUMAX-CD20M), an IgG1 human MAb that binds to a different CD20 epitope than rituximab; (2) veltuzumab (IMMUN-106™ or hA20), a humanized engineered antibody with complementarity-determining regions (CDRs) of murine origin and with 90% of the human framework regions identical to epratuzumab (a humanized anti-CD22 IgG1 antibody); (3) a small, modular immunopharmaceutical (SMIP) (herein called immunopharmaceutical) having SEQ ID NO:11 (also known as TRU-015); (4) a CD20-binding molecule that is an antibody comprising SEQ ID NOS:13 and 15 (Lilly AME 33) or SEQ ID NOS:17 and 18 (Lilly AME 133) or SEQ ID NO:19 (Lilly AME 133v, otherwise known as LY2469298, which binds with an increased affinity to the FcγRIIIa (CD16)); and (5) a humanized type II anti-CD20 antibody of the isotype IgG1 with a glycoengineered Fc portion (bisected afucosylated carbohydrates in the Fc region) and a modified elbow hinge, known as GA101. All of these antibodies are further described below, including the full-length or variable-region sequences thereof and defining literature.

The “variable region” or “variable domain” of an antibody refers to the amino-terminal domain of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light-chain and the heavy-chain variable domains. The more highly conserved portions of variable domains are called the framework regions (FR). The variable domains of native heavy and light chains each comprise four FR regions, largely adopting a β-sheet configuration, connected by three HVRs, which form loops connecting, and in some cases forming part of, the β-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in ADCC.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG1, IgG₂, IgG₃, IgG₄, IgA₁, and IgA₂. The heavy-chain constant domains that correspond to the different classes of immunoglobulins are called α, δ, ε, γ, and μ, respectively. The subunit structures and three-dimensional configurations of different classes of immunoglobulins are well known and described generally in, for example, Abbas et al. Cellular and Mol. Immunology, 4th ed. (W. B. Saunders, Co., 2000). An antibody may be part of a larger fusion molecule, formed by covalent or non-covalent association of the antibody with one or more other proteins or peptides.

The terms “full-length antibody,” “intact antibody,” and “whole antibody” are used herein interchangeably to refer to an antibody in its substantially intact form, not antibody fragments as defined below. The terms particularly refer to an antibody with heavy chains that contain an Fc region.

A “naked antibody” for the purposes herein is an antibody that is not conjugated to a cytotoxic moiety or radiolabel.

“Antibody fragments” comprise a portion of an intact antibody, preferably comprising the antigen-binding region thereof. Examples of antibody fragments include Fab, Fab′, F(ab′)₂, and Fv fragments; diabodies; linear antibodies; single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Fv” is the minimum antibody fragment that contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three HVRs of each variable domain interact to define an antigen-binding site on the surface of the VH-VL dimer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain, including one or more cysteines from the antibody-hinge region. Fab′-SH is the designation herein for Fab′, in which the cysteine residue(s) of the constant domains bear a free thiol group. F(ab′)₂ antibody fragments originally were produced as pairs of Fab′ fragments that have hinge cysteines between them. Other chemical couplings of antibody fragments are also known.

“Single-chain Fv” or “scFv” antibody fragments comprise the VH and VL domains of an antibody, wherein these domains are present in a single polypeptide chain. Generally, the scFv polypeptide further comprises a polypeptide linker between the VH and VL domains that enables the scFv to form the desired structure for antigen binding. For a review of scFv, see, e.g., Pluckthün, in The Pharmacology of Monoclonal Antibodies, vol. 113, Rosenburg and Moore eds. (Springer-Verlag, New York: 1994), pp 269-315.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO 1993/01161; Hudson et al., Nat. Med., 9:129-134 (2003); and Hollinger et al., Proc. Natl. Acad. Sci. USA, 90: 6444-6448 (1993). Triabodies and tetrabodies are also described in Hudson et al., Nat. Med., 9:129-134 (2003).

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target-binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal-antibody preparation is directed against a single determinant on an antigen. In addition to their specificity, monoclonal-antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein., Nature, 256:495-97 (1975); Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas, 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol., 222: 581-597 (1992); Sidhu et al., J. Mol. Biol., 338(2): 299-310 (2004); Lee et al., J. Mol. Biol., 340(5): 1073-1093 (2004); Fellouse, Proc. Natl. Acad. Sci. USA, 101(34): 12467-12472 (2004); and Lee et al., J. Immunol. Methods, 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA, 90: 2551 (1993); Jakobovits et al., Nature, 362: 255-258 (1993); Bruggemann et al., Year in Immunol., 7:33 (1993); U.S. Pat. Nos. 5,545,807; 5,545,806; 5,569,825; 5,625,126; 5,633,425; and 5,661,016; Marks et al., Bio/Technology, 10: 779-783 (1992); Lonberg et al., Nature, 368: 856-859 (1994); Morrison, Nature, 368: 812-813 (1994); Fishwild et al., Nature Biotechnol., 14: 845-851 (1996); Neuberger, Nature Biotechnol., 14: 826 (1996); and Lonberg and Huszar, Intern. Rev. Immunol., 13: 65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (e.g., U.S. Pat. No. 4,816,567 and Morrison et al., Proc. Natl. Acad. Sci. USA, 81:6851-6855 (1984)). Chimeric antibodies include PRIMATIZED® antibodies wherein the antigen-binding region of the antibody is derived from an antibody produced by, e.g., immunizing macaque monkeys with the antigen of interest.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a HVR of the recipient are replaced by residues from a HVR of a non-human species (donor antibody) such as mouse, rat, rabbit, or non-human primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all, or substantially all, of the FRs are those of a human immunoglobulin sequence. The humanized antibody optionally will also comprise at least a portion of an immunoglobulin constant region (Fc), typically that of a human immunoglobulin. For further details, see, e.g., Jones et al., Nature, 321:522-525 (1986); Riechmann et al., Nature, 332:323-329 (1988); and Presta, Curr. Op. Struct. Biol., 2:593-596 (1992). See also, for example, Vaswani and Hamilton, Ann. Allergy, Asthma & Immunol., 1: 105-115 (1998); Harris, Biochem. Soc. Transactions, 23:1035-1038 (1995); Hurle and Gross, Curr. Op. Biotech., 5:428-433 (1994); and U.S. Pat. Nos. 6,982,321 and 7,087,409.

A “human antibody” is one that possesses an amino-acid sequence that corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

The term “hypervariable region,” “HVR,” or “HV,” when used herein refers to the regions of an antibody-variable domain that are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity, 13:37-45 (2000) and Johnson and Wu in Methods in Molecular Biology, 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature, 363:446-448 (1993) and Sheriff et al., Nature Struct. Biol., 3:733-736 (1996).

A number of HVR delineations are in use and are encompassed herein. The HVRs that are Kabat complementarity-determining regions (CDRs) are based on sequence variability and are the most commonly used (Kabat et al., supra). Chothia refers instead to the location of the structural loops (Chothia and Lesk, J. Mol. Biol., 196:901-917 (1987)). The AbM HVRs represent a compromise between the Kabat CDRs and Chothia structural loops, and are used by Oxford Molecular's AbM antibody-modeling software. The “contact” HVRs are based on an analysis of the available complex crystal structures. The residues from each of these HVRs are noted below.

| Loop | Kabat | AbM | Chothia | Contact | |
|---|---|---|---|---|---|
| L1 | L24-L34 | L24-L34 | L26-L32 | L30-L36 | |
| L2 | L50-L56 | L50-L56 | L50-L52 | L46-L55 | |
| L3 | L89-L97 | L89-L97 | L91-L96 | L89-L96 | |
| H1 | H31-H35B | H26-H35B | H26-H32 | H30-H35B | (Kabat Numbering) |
| H1 | H31-H35 | H26-H35 | H26-H32 | H30-H35 | (Chothia Numbering) |
| H2 | H50-H65 | H50-H58 | H53-H55 | H47-H58 | |
| H3 | H95-H102 | H95-H102 | H96-H101 | H93-H101 | |

HVRs may comprise “extended HVRs” as follows: 24-36 or 24-34 (L1), 46-56 or 50-56 (L2), and 89-97 or 89-96 (L3) in the VL, and 26-35 (H1), 50-65 or 49-65 (H2), and 93-102, 94-102, or 95-102 (H3) in the VH. The variable-domain residues are numbered according to Kabat et al., supra, for each of these extended-HVR definitions.

“Framework” or “FR” residues are those variable-domain residues other than the HVR residues as herein defined.

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino-acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat-numbered sequence.

An “affinity-matured” antibody is one with one or more alterations in one or more HVRs thereof that result in an improvement in the affinity of the antibody for antigen, compared to a parent antibody that does not possess those alteration(s). In one embodiment, an affinity-matured antibody has nanomolar or even picomolar affinities for the target antigen. Affinity-matured antibodies are produced by procedures known in the art. For example, Marks et al., Bio/Technology, 10:779-783 (1992) describes affinity maturation by VH- and VL-domain shuffling. Random mutagenesis of HVR and/or framework residues is described by, for example: Barbas et al., Proc Nat. Acad. Sci. USA, 91:3809-3813 (1994); Schier et al., Gene, 169:147-155 (1995); Yelton et al., J. Immunol., 155:1994-2004 (1995); Jackson et al., J. Immunol., 154(7):3310-9 (1995); and Hawkins et al., J. Mol. Biol., 226:889-896 (1992).

“Growth-inhibitory” antibodies are those that prevent or reduce proliferation of a cell expressing an antigen to which the antibody binds. For example, the antibody may prevent or reduce proliferation of B cells in vitro and/or in vivo.

Antibodies that “induce apoptosis” are those that induce programmed cell death, e.g. of a B cell, as determined by standard apoptosis assays, such as binding of annexin V, fragmentation of DNA, cell shrinkage, dilation of endoplasmic reticulum, cell fragmentation, and/or formation of membrane vesicles (called apoptotic bodies).

Antibody “effector functions” refer to those biological activities attributable to the Fc region (a native-sequence Fc region or amino-acid-sequence-variant Fc region) of an antibody, and vary with the antibody isotype. Examples of antibody effector functions include: C1q binding and CDC; Fc-receptor binding; ADCC; phagocytosis; down-regulation of cell-surface receptors (e.g., B-cell receptor); and B-cell activation.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue.

Unless indicated otherwise herein, the numbering of the residues in an immunoglobulin heavy chain is that of the EU index as in Kabat et al., supra. The “EU index as in Kabat” refers to the residue numbering of the human IgG1 EU antibody.

A “functional Fc region” possesses an “effector function” of a native-sequence Fc region. Exemplary “effector functions” include C1q binding; CDC; Fc-receptor binding; ADCC; phagocytosis; down-regulation of cell-surface receptors (e.g., B-cell receptor), etc. Such effector functions generally require the Fc region to be combined with a binding domain (e.g. an antibody-variable domain) and can be assessed using various assays as disclosed, for example, in definitions herein.

A “native-sequence Fc region” comprises an amino acid sequence identical to the amino acid sequence of an Fc region found in nature. Native-sequence human Fc regions include a native-sequence human IgG1 Fc region (non-A and A allotypes); native-sequence human IgG2 Fc region; native-sequence human IgG3 Fc region; and native-sequence human IgG4 Fc region, as well as naturally occurring variants thereof.

A “variant Fc region” comprises an amino acid sequence that differs from that of a native-sequence Fc region by virtue of at least one amino acid modification, preferably one or more amino acid substitution(s). Preferably, the variant Fc region has at least one amino acid substitution compared to a native-sequence Fc region or to the Fc region of a parent polypeptide, e.g., from about one to about ten amino acid substitutions, and preferably from about one to about five amino acid substitutions in a native-sequence Fc region or in the Fc region of the parent polypeptide. The variant Fc region herein will preferably possess at least about 80% homology with a native-sequence Fc region and/or with an Fc region of a parent polypeptide, and most preferably at least about 90% homology therewith, more preferably at least about 95% homology therewith.

The term “Fc-region-comprising antibody” refers to an antibody that comprises an Fc region. The C-terminal lysine (residue 447 according to the EU numbering system) of the Fc region may be removed, for example, during purification of the antibody or by recombinant engineering the nucleic acid encoding the antibody. Accordingly, a composition comprising an antibody having an Fc region according to this invention can comprise an antibody with K447, with all K447 removed, or a mixture of antibodies with and without the K447 residue.

“Fc receptor” or “FcR” describes a receptor that binds to the Fc region of an antibody. In some embodiments, an FcR is a native-human FcR. In some embodiments, an FcR is one that binds an IgG antibody (a gamma receptor) and includes receptors of the FcγRI, FcγRII, and FcγRIII subclasses, including allelic variants and alternatively spliced forms of those receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see, e.g., Daëron, Annu. Rev. Immunol., 15:203-234 (1997)). FcRs are reviewed, for example, in Ravetch and Kinet, Annu. Rev. Immunol, 9:457-92 (1991); Capel et al., Immunomethods, 4:25-34 (1994); and de Haas et al., J. Lab. Clin. Med., 126:330-41 (1995).

The term “Fc receptor” or “FcR” also includes the neonatal receptor, FcRn, which is responsible for the transfer of maternal IgGs to the fetus (Guyer et al., J. Immunol., 117:587 (1976) and Kim et al., J. Immunol., 24:249 (1994)) and regulation of homeostasis of immunoglobulins. Methods of measuring binding to FcRn are known (see, e.g., Ghetie and Ward, Immunology Today, 18 (12):592-598 (1997); Ghetie et al., Nature Biotechnology, 15 (7):637-640 (1997); Hinton et al., J. Biol. Chem., 279(8):6213-6216 (2004); and WO 2004/92219 (Hinton et al.)).

Binding to human FcRn in vivo and serum half-life of human FcRn high-affinity binding polypeptides can be assayed, e.g., in transgenic mice or transfected human cell lines expressing human FcRn, or in primates to which the polypeptides with a variant Fc region are administered. WO 2000/42072 (Presta) describes antibody variants with improved or diminished binding to FcRs. See, also, for example, Shields et al., J. Biol. Chem., 9(2): 6591-6604 (2001).

“Human effector cells” are leukocytes that express one or more FcRs and perform effector functions. In certain embodiments, the cells express at least FcγRIII and perform ADCC effector function(s). Examples of human leukocytes that mediate ADCC include peripheral blood mononuclear cells (PBMC), natural-killer (NK) cells, monocytes, cytotoxic T cells, and neutrophils. The effector cells may be isolated from a native source, e.g., from blood.

“Antibody-dependent cell-mediated cytotoxicity” or “ADCC” refers to a form of cytotoxicity in which secreted Ig bound onto Fc receptors (FcRs) present on certain cytotoxic cells (e.g., NK cells, neutrophils, and macrophages) enables these cytotoxic effector cells to bind specifically to an antigen-bearing target cell and subsequently kill the target cell with cytotoxins. The primary cells for mediating ADCC, NK cells, express FcγRIII only, whereas monocytes express FcγRI, FcγRII, and FcγRIII. FcR expression on hematopoietic cells is summarized in Table 3 on page 464 of Ravetch and Kinet, Annu. Rev. Immunol., 9:457-492 (1991). To assess ADCC activity of a molecule of interest, an in vitro ADCC assay, such as that described in U.S. Pat. No. 5,500,362, 5,821,337 or 6,737,056 may be performed. Useful effector cells for such assays include PBMC and NK cells. Alternatively, or additionally, ADCC activity of the molecule of interest may be assessed in vivo, e.g., in an animal model such as that disclosed in Clynes et al., Proc. Natl. Acad. Sci. USA, 95:652-656 (1998).

“Complement-dependent cytotoxicity” or “CDC” refers to the lysis of a target cell in the presence of complement. Activation of the classical complement pathway is initiated by the binding of the first component of the complement system (C1q) to antibodies (of the appropriate subclass), which are bound to their cognate antigen. To assess complement activation, a CDC assay, e.g., as described in Gazzano-Santoro et al., J. Immunol. Methods, 202:163 (1996), may be performed. Polypeptide variants with altered Fc-region amino acid sequences (polypeptides with a variant Fc region) and increased or decreased C1q binding capability are described, e.g., in U.S. Pat. No. 6,194,551 and WO 1999/51642. See, also, e.g., Idusogie et al., J. Immunol., 164: 4178-4184 (2000).

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity that reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative and exemplary embodiments for measuring binding affinity are described in the following.

In one embodiment, the “Kd” or “Kd value” according to this invention is measured by a radiolabeled antigen-binding assay (RIA) performed with the Fab version of an antibody of interest and its antigen as described by the following assay. Solution-binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol., 293:865-881 (1999)). To establish conditions for the assay, microtiter plates (DYNEX Technologies, Inc.) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate, 100 pM or 26 pM [¹²⁵I]-antigen is mixed with serial dilutions of a Fab of interest (e.g., consistent with assessment of the anti-VEGF antibody, Fab-12, in Presta et al., Cancer Res., 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% TWEEN-20™ surfactant in PBS. When the plates have dried, 150 μl/well of scintillant (MICROSCINT-20; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, the Kd or Kd value is measured by surface-plasmon resonance assays using a BIACORE®-2000 or a BIACORE®-3000 instrument (BIAcore, Inc., Piscataway, N.J.) at 25° C. with immobilized antigen CM5 chips at ˜10 response units (RU). Briefly, carboxymethylated dextran biosensor chips (CM5, BIAcore Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately ten RU of coupled protein. Following the injection of antigen, 1 M ethanolamine is injected to block unreacted groups. For kinetics measurements, two-fold serial dilutions of Fab (0.78 nM to 500 nM) are injected in PBS with 0.05% TWEEN 20™ surfactant (PBST) at 25° C. at a flow rate of approximately 25 μl/min. Association rates (kₒₙ) and dissociation rates (k_{off}) are calculated using a simple one-to-one Langmuir binding model (BIAcore® Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol., 293:865-881 (1999). If the on-rate exceeds 10⁶ M⁻¹s⁻¹ by the surface-plasmon resonance assay above, then the on-rate can be determined by using a fluorescent quenching technique that measures the increase or decrease in fluorescence-emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow-equipped spectrophotometer (Aviv Instruments) or a 8000-series SLM-AMINCO™ spectrophotometer (ThermoSpectronic) with a stirred cuvette.

An “on-rate,” “rate of association,” “association rate,” or “kₒₙ” according to this invention can also be determined as described above using a BIACORE®-2000 or a BIACORE®-3000 system (BIAcore, Inc., Piscataway, N.J.).

The term “substantially similar” or “substantially the same,” as used herein, denotes a sufficiently high degree of similarity between two numeric values (for example, one associated with an antibody of the invention and the other associated with a reference/comparator antibody), such that one of skill in the art would consider the difference between the two values to be of little or no biological and/or statistical significance within the context of the biological characteristic measured by said values (e.g., Kd values). The difference between said two values is, for example, less than about 50%, less than about 40%, less than about 30%, less than about 20%, and/or less than about 10% as a function of the reference/comparator value.

The phrase “substantially reduced,” or “substantially different,” as used herein, denotes a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., Kd values). The difference between said two values is, for example, greater than about 10%, greater than about 20%, greater than about 30%, greater than about 40%, and/or greater than about 50% as a function of the value for the reference/comparator molecule.

The terms “rituximab” or “RITUXAN®” herein refer to the genetically engineered chimeric murine/human monoclonal antibody directed against the CD20 antigen and designated “C2B8” in U.S. Pat. No. 5,736,137, including fragments thereof that retain the ability to bind CD20.

Purely for the purposes herein and unless indicated otherwise, “2H7” or “2H7 antibody” refers to a humanized anti-CD20 antibody with the sequences provided immediately below and/or described in US 2006/0034835 and WO 2004/056312 (both Lowman et al.); US 2006/0188495 (Barron et al.); and US 2006/0246004 (Adams et al.). Briefly, humanization of the murine anti-human CD20 antibody, 2H7 (also referred to herein as m2H7, m for murine), was carried out in a series of site-directed mutagenesis steps. The murine 2H7 antibody variable region sequences and the chimeric 2H7 with the mouse V and human C have been described, e.g., in U.S. Pat. Nos. 5,846,818 and 6,204,023. The CDR residues of 2H7 were identified by comparing the amino acid sequence of the murine 2H7 variable domains (disclosed in U.S. Pat. No. 5,846,818) with the sequences of known antibodies (Kabat et al., Sequences of Proteins of Immunological Interest, Ed. 5 (Public Health Service, National Institutes of Health, Bethesda, Md., 1991)). The CDRs for the light and heavy chains were defined based on sequence hypervariability (Kabat et al., supra). With synthetic oligonucleotides, site-directed mutagenesis (Kunkel, Proc. Natl. Acad. Sci. USA, 82:488-492 (1985)) was used to introduce all six of the murine 2H7CDRs into a complete human Fab framework corresponding to a consensus sequence V_{κ}I, V_{H}III (V_{L} kappa subgroup I, V_{H} subgroup E) contained on plasmid pVX4 (see FIG. 2 in WO 2004/056312). Further modifications of the V regions (CDR and/or FR) were made in the phagemid pVX4 by site-directed mutagenesis. Plasmids for expression of full-length IgG's were constructed by subcloning the V_{L} and V_{H} domains of chimeric 2H7Fab as well as humanized Fab versions 2 to 6 into previously described pRK vectors for mammalian cell expression (Gorman et al., DNA Prot. Eng. Tech., 2:3-10 (1990)).

The following 2H7 antibodies are included within the definition herein:

(1) A humanized antibody comprising the VL sequence:

(SEQ ID NO:24)

DIQMTQSPSSLSASVGDRVTITCRASSSVSYMHWYQQKPGKAPKPLIYAP
SNLASGVPSRFSGSGSGTDFTLTISSLQPEDFATYYCQQWSFNPPTFGQG
TKVEIKR;

and the VH sequence:

(SEQ ID NO:25)

EVQLVESGGGLVQPGGSLRLSCAASGYTFTSYNMHWVRQAPGKGLEWVGA
IYPGNGDTSYNQKFKGRFTISVDKSKNTLYLQMNSLRAEDTAVYYCARVV
YYSNSYWYFDVWGQGTLVTVSS.

(2) A humanized antibody comprising the VL sequence:

(SEQ ID NO:26)

DIQMTQSPSSLSASVGDRVTITCRASSSVSYLHWYQQKPGKAPKPLIYAP
SNLASGVPSRFSGSGSGTDFTLTISSLQPEDFATYYCQQWAFNPPTFGQG
TKVEIKR;

and the VH sequence:

(SEQ ID NO:27)

EVQLVESGGGLVQPGGSLRLSCAASGYTFTSYNMHWVRQAPGKGLEWVGA
IYPGNGATSYNQKFKGRFTISVDKSKNTLYLQMNSLRAEDTAVYYCARVV
YYSASYWYFDVWGQGTLVTVSS.

(3) A humanized antibody comprising the VL sequence:

(SEQ ID NO:26)

DIQMTQSPSSLSASVGDRVTITCRASSSVSYLHWYQQKPGKAPKPLIYAP
SNLASGVPSRFSGSGSGTDFTLTISSLQPEDFATYYCQQWAFNPPTFGQG
TKVEIKR;

and the VH sequence:

(SEQ ID NO:28)

EVQLVESGGGLVQPGGSLRLSCAASGYTFTSYNMHWVRQAPGKGLEWVGA
IYPGNGATSYNQKFKGRFTISVDKSKNTLYLQMNSLRAEDTAVYYCARVV
YYSYRYWYFDVWGQGTLVTVSS.

(4) A humanized antibody comprising a full-length light (L) chain having the sequence of SEQ ID NO:29, and a full-length heavy (H) chain having the sequence of one of SEQ ID NO:30, SEQ ID NO:31, or SEQ ID NO:38, wherein the sequences are indicated below.
(5) A humanized antibody comprising a full-length light (L) chain having the sequence of SEQ ID NO:32, and a full-length heavy (H) chain having the sequence of one of SEQ ID NO:33, SEQ ID NO:34, SEQ ID NO:35, SEQ ID NO:36, or SEQ ID NO:37, wherein the sequences are indicated below.

SEQ ID NO:29:
DIQMTQSPSSLSASVGDRVTITCRASSSVSYMHWYQQKPGKAPKPLIYAP
SNLASGVPSRFSGSGSGTDFTLTISSLQPEDFATYYCQQWSFNPPTFGQG
TKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC
SEQ ID NO: 30:
EVQLVESGGGLVQPGGSLRLSCAASGYTFTSYNMHWVRQAPGKGLEWVGA
IYPGNGDTSYNQKFKGRETISVDKSKNTLYLQMNSLRAEDTAVYYCARVV
YYSNSYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
G
SEQ ID NO:31:
EVQLVESGGGLVQPGGSLRLSCAASGYTFTSYNMHWVRQAPGKGLEWVGA
IYPGNGDTSYNQKFKGRFTISVDKSKNTLYLQMNSLRAEDTAVYYCARVV
YYSNSYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCKVSNKALPAPIAATISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
G
SEQ ID NO:32:
DIQMTQSPSSLSASVGDRVTITCRASSSVSYLHWYQQKPGKAPKPLIYAP
SNLASGVPSRFSGSGSGTDFTLTISSLQPEDFATYYCQQWAFNPPTFGQG
TKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC
SEQ ID NO:33:
EVQLVESGGGLVQPGGSLRLSCAASGYTFTSYNMHWVRQAPGKGLEWVGA
IYPGNGATSYNQKFKGRFTISVDKSKNTLYLQMNSLRAEDTAVYYCARVV
YYSASYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCKVSNKALPAPIAATISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
G
SEQ ID NO:34:
EVQLVESGGGLVQPGGSLRLSCAASGYTFTSYNMHWVRQAPGKGLEWVGA
IYPGNGATSYNQKFKGRFTISVDKSKNTLYLQMNSLRAEDTAVYYCARVV
YYSASYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCAVSNKALPAPIEATISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
G
SEQ ID NO:35:
EVQLVESGGGLVQPGGSLRLSCAASGYTFTSYNMHWVRQAPGKGLEWVGA
IYPGNGATSYNQKFKGRFTISVDKSKNTLYLQMNSLRAEDTAVYYCARVV
YYSASYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCKVSNAALPAPIAATISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
G
SEQ ID NO:36:
EVQLVESGGGLVQPGGSLRLSCAASGYTFTSYNMHWVRQAPGKGLEWVGA
IYPGNGATSYNQKFKGRFTISVDKSKNTLYLQMNSLRAEDTAVYYCARVV
YYSASYWYFDVWGQGTLVTVSSASTKGPSVFPLAPSSKSTSGGTAALGCL
VKDYEPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCKVSNAALPAPIAATLSKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHWHYTQKSLSLSP
G
SEQ ID NO:37:
EVQLVESGGGLVQPGGSLRLSCAASGYTFTSYNMHWVRQAPGKGLEWVGA
IYPGNGATSYNQKFKGRFTISVDKSKNTLYLQMNSLRAEDTAVYYCARVV
YYSYRYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKV EPKSCDKTHTCPPCPAPELLGGPSVFLFP
PKPKDTLMISRTPEVTCVVVDVSHEDPEVKFNWYVDGVEVHNAKTKPREE
QYNATYRVVSVLTVLHQDWLNGKEYKCKVSNAALPAPIAATISKAKGQPR
EPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLS
PG
SEQ ID NO:38:
EVQLVESGGGLVQPGGSLRLSCAASGYTFTSYNMHWVRQAPGKGLEWVGA
IYPGNGDTSYNQKFKGRFTISVDKSKNTLYLQMNSLRAEDTAVYYCARVV
YYSNSYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCKVSNAALPAPIAATISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
G

The murine anti-human CD20 antibody, m2H7 comprises the variable region sequences:

VL sequence:

(SEQ ID NO:39)

QIVLSQSPAI LSASPGEKVT MTCRASSSVS YMHWYQQKPG
SSPKPWIYAP SNLASGVPARFSGSGSGTSY SLTISRVEAE
DAATYYCQQWSFNPPTFGAG TKLELK
VH sequence:

(SEQ ID NO:40)

QAYLQQSGAELVRPGASVKMSCKASGYTFTSYNMHWVKQTPRQGLEWIGA
IYPGNGDTSYNQKFKGKATLTVDKSSSTAYMQLSSLTSEDSAVYFCARVV
YYSNSYWYFDVWGTGTTVTVS

In the anti-CD20 antibodies that comprise an Fc region, the C-terminal lysine (residue 447 according to the EU numbering system) of the Fc region may be removed, for example, during purification of the antibody or by recombinant engineering of the nucleic acid encoding the antibody polypeptide. For example, hA20 can comprise an Fc region including the K447 residue, or with all the K447 residues removed, or a mixture of antibodies having Fc regions with and without the K447 residue.

In certain embodiments, the anti-CD20 antibody useful herein further comprises amino acid alterations in the IgG Fc and exhibits increased binding affinity for human FcRn over an antibody having wild-type IgG Fc, by at least about 60 fold, preferably at least about 70 fold, more preferably at least about 80 fold, even more preferably at least about 100 fold, still more preferably at least about 125 fold, and most preferably at least about 150 fold to about 170 fold.

The N-glycosylation site in IgG is at Asn297 in the CH2 domain. Included for use in therapy herein are compositions of any eligible anti-CD20 antibodies herein having an Fc region, wherein about 80-100% (and preferably about 90-99%) of the antibody in the composition comprises a mature core carbohydrate structure that lacks fucose, attached to the Fc region of the glycoprotein, or has reduced fucose content.

“Mammal” for purposes of treatment refers to any animal classified as a mammal, including humans, domestic and farm animals, and zoo, sports, or pet animals, such as dogs, horses, cats, cows, etc. Preferably, the mammal is human.

The expression “effective amount” refers to an amount of a medicament that is effective for treating RA or joint damage. This would include an amount that is effective in achieving a reduction in RA or joint damage as compared to baseline prior to administration of such amount as determined, e.g., by radiographic or other testing. An effective amount of a second medicament may serve not only to treat the RA or joint damage in conjunction with an anti-CD20 antibody herein, but also serve to treat undesirable effects, including side-effects or symptoms or other conditions accompanying RA or joint damage, including a concomitant or underlying disease or disorder.

As used herein, “rheumatoid arthritis” or “RA” refers to a recognized disease state that may be diagnosed according to the 2000 revised American Rheumatoid Association criteria for the classification of RA, or any similar criteria. The term includes not only active and early RA, but also incipient RA, as defined below. Physiological indicators of RA include symmetric joint swelling that is characteristic though not invariable in RA. Fusiform swelling of the proximal interphalangeal (PIP) joints of the hands as well as metacarpophalangeal (MCP), wrists, elbows, knees, ankles, and metatarsophalangeal (MTP) joints are commonly affected and swelling is easily detected. Pain on passive motion is the most sensitive test for joint inflammation, and inflammation and structural deformity often limits the range of motion for the affected joint. Typical visible changes include ulnar deviation of the fingers at the MCP joints, hyperextension, or hyperflexion of the MCP and PIP joints, flexion contractures of the elbows, and subluxation of the carpal bones and toes. In addition to being resistant to rituximab, the subject with RA may be resistant to DMARDs, in that the DMARDs are not effective or fully effective in treating symptoms, or may have experienced an inadequate response to previous or current treatment with TNF-α inhibitors such as etanercept, infliximab and/or adalimumab because of toxicity or inadequate efficacy (for example, etanercept for 3 months at 25 mg twice a week or at least 4 infusions of infliximab at 3 mg/kg).

A patient with “active rheumatoid arthritis” means a patient with active and not latent symptoms of RA. Subjects with “early active rheumatoid arthritis” are those subjects with active RA diagnosed for at least 8 weeks but no longer than four years, according to the revised 1987 ACR criteria for the classification of RA.

Subjects with “early rheumatoid arthritis” are those subjects with RA diagnosed for at least eight weeks but no longer than four years, according to the revised 1987 ACR criteria for classification of RA. RA includes, for example, juvenile-onset RA, juvenile idiopathic arthritis (JIA), or juvenile RA (JRA).

Patients with “incipient RA” have early polyarthritis that does not fully meet ACR criteria for a diagnosis of RA. They include patients with positive anti-cyclic citrullinated peptide (anti-CCP) antibodies who present with polyarthritis, but do not yet have a diagnosis of RA, and are at high risk for going on to develop bona fide ACR criteria RA (95% probability).

“Joint damage” is used in the broadest sense and refers to damage or partial or complete destruction to any part of one or more joints, including the connective tissue and cartilage, where damage includes structural and/or functional damage of any cause, and may or may not cause joint pain/arthalgia. It includes, without limitation, joint damage associated with or resulting from inflammatory as well as non-inflammatory joint disease. This damage may be caused by any condition, such as an autoimmune disease, especially arthritis, and most especially RA. Exemplary such conditions include acute and chronic arthritis, RA including juvenile-onset RA, JIA, or JRA, and stages such as rheumatoid synovitis, gout or gouty arthritis, acute immunological arthritis, chronic inflammatory arthritis, degenerative arthritis, type II collagen-induced arthritis, infectious arthritis, septic arthritis, Lyme arthritis, proliferative arthritis, psoriatic arthritis, Still's disease, vertebral arthritis, osteoarthritis, arthritis chronica progrediente, arthritis deformans, polyarthritis chronica primaria, reactive arthritis, menopausal arthritis, estrogen-depletion arthritis, and ankylosing spondylitis/rheumatoid spondylitis), rheumatic autoimmune disease other than RA, and significant systemic involvement secondary to RA (including but not limited to vasculitis, pulmonary fibrosis or Felty's syndrome). For purposes herein, joints are points of contact between elements of a skeleton (of a vertebrate such as an animal) with the parts that surround and support it and include, but are not limited to, for example, hips, joints between the vertebrae of the spine, joints between the spine and pelvis (sacroiliac joints), joints where the tendons and ligaments attach to bones, joints between the ribs and spine, shoulders, knees, feet, elbows, hands, fingers, ankles, and toes, but especially joints in the hands and feet.

An “autoimmune disease” herein is a disease or disorder arising from and directed against an individual's own tissues or organs or a co-segregate or manifestation thereof or resulting condition therefrom. In many of these autoimmune and inflammatory disorders, a number of clinical and laboratory markers may exist, including, but not limited to, hypergammaglobulinemia, high levels of autoantibodies, antigen-antibody complex deposits in tissues, benefit from corticosteroid or immunosuppressive treatments, and lymphoid cell aggregates in affected tissues. Without being limited to any one theory regarding B-cell mediated autoimmune disease, it is believed that B cells demonstrate a pathogenic effect in human autoimmune diseases through a multitude of mechanistic pathways, including autoantibody production, immune complex formation, dendritic and T-cell activation, cytokine synthesis, direct chemokine release, and providing a nidus for ectopic neo-lymphogenesis. Each of these pathways may participate to different degrees in the pathology of autoimmune diseases.

The “autoimmune disease” herein can be an organ-specific disease (i.e., the immune response is specifically directed against an organ system such as the endocrine system, the hematopoietic system, the skin, the cardiopulmonary system, the gastrointestinal and liver systems, the renal system, the thyroid, the ears, the neuromuscular system, the central nervous system, etc.) or a systemic disease which can affect multiple organ systems (for example, SLE, RA, polymyositis, etc.). Preferred such diseases include autoimmune rheumatologic disorders (such as, for example, RA, Sjögren's syndrome, scleroderma, lupus such as SLE and lupus nephritis, polymyositis/dermatomyositis, cryoglobulinemia, anti-phospholipid antibody syndrome, and psoriatic arthritis), autoimmune gastrointestinal and liver disorders (such as, for example, inflammatory bowel diseases (e.g., ulcerative colitis and Crohn's disease), autoimmune gastritis and pernicious anemia, autoimmune hepatitis, primary biliary cirrhosis, primary sclerosing cholangitis, and celiac disease), vasculitis (such as, for example, ANCA-negative vasculitis and AAV, including Churg-Strauss vasculitis, Wegener's granulomatosis, and microscopic polyangiitis), autoimmune neurological disorders (such as, for example, MS, opsoclonus myoclonus syndrome, myasthenia gravis, neuromyelitis optica, Parkinson's disease, Alzheimer's disease, and autoimmune polyneuropathies), renal disorders (such as, for example, glomerulonephritis, Goodpasture's syndrome, and Berger's disease), autoimmune dermatologic disorders (such as, for example, psoriasis, urticaria, hives, pemphigus vulgaris, bullous pemphigoid, and cutaneous lupus erythematosus), hematologic disorders (such as, for example, thrombocytopenic purpura, thrombotic thrombocytopenic purpura, post-transfusion purpura, and autoimmune hemolytic anemia), atherosclerosis, uveitis, autoimmune hearing diseases (such as, for example, inner ear disease and hearing loss), Behcet's disease, Raynaud's syndrome, organ transplant, and autoimmune endocrine disorders (such as, for example, diabetic-related autoimmune diseases such as insulin-dependent diabetes mellitus (IDDM), Addison's disease, and autoimmune thyroid disease (e.g., Graves' disease and thyroiditis)). More preferred such diseases include, for example, RA, ulcerative colitis, AAV, lupus, MS, Sjögren's syndrome, Graves' disease, IDDM, pernicious anemia, thyroiditis, and glomerulonephritis.

“Treatment” of a subject herein refers to both therapeutic treatment and prophylactic or preventative measures. Those in need of treatment include those already with RA or joint damage as well as those in which the RA or joint damage or the progress of RA or joint damage is to be prevented. Hence, the subject may have been diagnosed as having the RA or joint damage or may be predisposed or susceptible to the RA or joint damage, or may have RA or joint damage that is likely to progress in the absence of treatment. Treatment is successful herein if the RA or joint damage is alleviated or healed, or progression of RA or joint damage, including its signs and symptoms and structural damage, is halted or slowed down as compared to the condition of the subject prior to administration.

Successful treatment further includes complete or partial prevention of RA or of the development of joint or structural damage. For purposes herein, slowing down or reducing RA or joint damage or the progression of joint damage is the same as arrest, decrease, or reversal of the RA or joint damage. The effectiveness of treatment of RA in the method can, for example, be determined by using the ACR and/or European League Against Rheumatism (EULAR) clinical response parameters in the patients with RA, or by assaying a molecular determinant of the degree of RA in the patient.

A clinician may use any of several methods known in the art to measure the effectiveness of a particular dosage scheme of an anti-CD20 antibody herein. For example, x-ray technology can be used to determine the extent of joint destruction and damage in the patient, and the scale of ACR20, ACR50, and ACR70 can be used to determine relative effective responsiveness to the therapy. Dosage regimens may be adjusted to provide the optimum desired response (e.g., a therapeutic response). For example, a dose may be administered, several divided doses may be administered over time or the dose may be proportionally reduced or increased as indicated by exigencies of the therapeutic situation.

An “effective response” of a patient or a patient's “responsiveness” to treatment with an anti-CD20 antibody herein and similar wording refers to the clinical or therapeutic benefit imparted to a RA patient not responsive to rituximab from or as a result of the treatment with an anti-CD20 antibody herein. Such benefit includes cellular or biological responses, a complete response, a partial response, a stable disease (without progression or relapse), or a response with a later relapse of the patient from or as a result of the treatment with an anti-CD20 antibody herein. For example, an effective response can be a higher ACR50 in a patient.

A “subject” herein is any single human subject, including a patient, eligible for treatment that is experiencing or has experienced one or more signs, symptoms, or other indicators of RA or joint damage, whether, for example, newly diagnosed or previously diagnosed and now experiencing a non-response, such as a recurrence or relapse, from rituximab treatment. Intended to be included as a subject are any subjects involved in clinical research trials. The subject may be naïve to a second medicament being used when the treatment herein is started, i.e., the subject may not have been previously treated with, for example, an immunosuppressive agent such as methotrexate (MTX) at “baseline” (i.e., at a set point in time before the administration of a first dose of one of the anti-CD20 antibodies in the treatment method herein, such as the day of screening the subject before treatment is commenced). Such “naïve” subjects are generally considered to be candidates for treatment with such second medicament.

“Clinical improvement” refers to prevention of further progress of RA or joint damage or any improvement in RA or joint damage as a result of treatment, as determined by various testing, including radiographic testing. Thus, clinical improvement may, for example, be determined by assessing the number of tender or swollen joints, the Psoriasis Assessment Severity Index, a global clinical assessment of the subject, assessing erythrocyte sedimentation rate, or assessing the amount of C-reactive protein level.

For purposes herein, a subject is in “remission” if he/she has no symptoms of RA or active joint damage, such as those detectable by the methods disclosed herein, and has had no progression of RA or joint damage as assessed at baseline or at a certain point of time during treatment. Those who are not in remission include, for example, those experiencing a worsening or progression of RA or joint damage. Such subjects experiencing a return of symptoms, including active RA or joint damage, are those who are “non-responsive” or have “relapsed” or had a “recurrence.”

The expression “not responsive to,” as it relates to the reaction of subjects or patients to rituximab (or other drugs to which they are not responsive as set forth herein), describes those subjects or patients who, upon administration of rituximab (or such other drugs), did not exhibit any or adequate signs of treatment of the RA, or they exhibited a clinically unacceptably high degree of toxicity to the rituximab (or other drugs), or they did not maintain the signs of treatment after first being administered the rituximab (or other drugs), with the word “treatment” being used in this context as defined herein. The phrase “not responsive” includes a description of those subjects who are resistant and/or refractory to the previously administered rituximab (or other drug), and includes the situations in which a subject or patient has progressed while receiving the rituximab (or other drug) that he or she is being given, and in which a subject or patient has progressed within 12 months (more preferably, within six months) after completing a regimen involving the rituximab (or other drug) to which he or she is no longer responsive.

The “non-responsiveness” to rituximab (or other drug) thus includes subjects who continue to have active disease following previous or current treatment therewith. For instance, a patient may have active disease activity after about one to three months of therapy with the rituximab (or other drug) to which they are non-responsive. Such responsiveness may be assessed by a clinician skilled in treating the disorder in question. The descriptor “non-responsive” also refers to responders to a first round of rituximab (or other drug), but not to a second or later course of such drug in a re-treatment regimen. It includes secondary non-responders who respond to a first clinical endpoint, but not to secondary endpoints. It includes those patients who have depleted B cells but do not respond to, for example, rituximab treatment, as well as patients who do not have depleted B cells. It also includes patients who show less than an about 20% improvement in ACR response, for example, at week 24 or at later weeks.

For purposes of non-response to medicament(s), a subject who experiences “a r clinically unacceptably high level of toxicity” from previous or current treatment with one or more medicaments (such as a TNF-alpha inhibitor) experiences one or more negative side-effects or adverse events associated therewith that are considered by an experienced clinician to be significant, such as, for example, immune reactions, including developing autoantibodies and human anti-chimeric antibodies (HACA), as determined by standard tests in the literature such as, e.g., enzyme-linked immunosorbent assays on serum (Prometheus Laboratories, San Diego, Calif.) (see, for example, Baert et al., N Engl J Med, 348:601-608 (2003), serious infections, congestive heart failure, demyelination (leading to MS), significant hypersensitivity, neuropathological events, high degrees of autoimmunity, a cancer such as endometrial cancer, NHL, tuberculosis, breast cancer, prostate cancer, lung cancer, ovarian cancer, melanoma, etc.

A “symptom” of RA or joint damage is any morbid phenomenon or departure from the normal in structure, function, or sensation, experienced by the subject and indicative of RA or joint damage, such as those noted above, including tender or swollen joints.

“Total modified Sharp score” means a score obtained for assessment of radiographs using the method according to Sharp, as modified by Genant, Am. J. Med., 30: 35-47 (1983). The primary assessment will be the change in the total Sharp-Genant score from screening. The Sharp-Genant score combines an erosion score and a joint space narrowing score of both hands and feet. Joint damage is measured in this test scoring by a mean change of less than the score at baseline (when the patient is screened or tested before first administration of an anti-CD20 antibody herein).

The term “cytotoxic agent” as used herein refers to a substance that inhibits or prevents the function of cells and/or causes destruction of cells. The term is intended to include radioactive isotopes (e.g., At211, I131, I125, Y90, Re186, Re188, Sm153, Bi212, P32, and radioactive isotopes of Lu), chemotherapeutic agents, and toxins such as small-molecule toxins or enzymatically active toxins of bacterial, fungal, plant or animal origin, or fragments thereof.

A “chemotherapeutic agent” is a chemical compound useful in the treatment of cancer. Examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethiylenethiophosphoramide, and trimethylolomelamine; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as MTX and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®, Rhône-Poulenc Rorer, Antony, France); chlorambucil; gemcitabine; 6-thioguanine; mercaptopurine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; XELODA® (capecitabine); ibandronate; CPT-11; topoisomerase inhibitor RFS 2000; difluoromethylomithine (DFMO); retinoic acid; esperamicins; capecitabine; and pharmaceutically acceptable salts, acids or derivatives of any of the above.

The term “immunosuppressive agent” as used herein for adjunct therapy refers to substances that act to suppress or mask the immune system of the mammal being treated herein. This would include substances that suppress cytokine production, down-regulate or suppress self-antigen expression, or mask the MHC antigens. Examples of such agents include 2-amino-6-aryl-5-substituted pyrimidines (see U.S. Pat. No. 4,665,077); NSAIDs; ganciclovir, tacrolimus, glucocorticoids such as cortisol or aldosterone, anti-inflammatory agents such as a cyclooxygenase inhibitor, a 5-lipoxygenase inhibitor, or a leukotriene receptor antagonist; purine antagonists such as azathioprine or mycophenolate mofetil (MMF); alkylating agents such as cyclophosphamide; bromocryptine; danazol; dapsone; glutaraldehyde (which masks the MHC antigens, as described in U.S. Pat. No. 4,120,649); anti-idiotypic antibodies for MHC antigens and MHC fragments; cyclosporin A; steroids such as corticosteroids or glucocorticosteroids or glucocorticoid analogs, e.g., prednisone, methylprednisolone, including SOLU-MEDROL® methylprednisolone sodium succinate, and dexamethasone; dihydrofolate reductase inhibitors such as MTX (oral or subcutaneous); anti-malarial agents such as chloroquine and hydroxychloroquine; sulfasalazine; leflunomide; cytokine antagonists such as cytokine antibodies or cytokine receptor antibodies including anti-interferon-α, -β, or -γ antibodies, anti-TNF-α antibodies (infliximab (REMICADE®) or adalimumab), anti-TNF-α immunoadhesin (etanercept), anti-TNF-β antibodies, anti-interleukin-2 (IL-2) antibodies and anti-IL-2 receptor antibodies, and anti-IL-6 receptor antibodies and antagonists (such as ACTEMRA™ (tocilizumab)); anti-LFA-1 antibodies, including anti-CD11a and anti-CD18 antibodies; anti-L3T4 antibodies; heterologous anti-lymphocyte globulin; pan-T antibodies, preferably anti-CD3 or anti-CD4/CD4a antibodies; soluble peptide containing a LFA-3 binding domain (WO 1990/08187); streptokinase; transforming growth factor-β (TGF-β); streptodornase; RNA or DNA from the host; FK506; RS-61443; chlorambucil; deoxyspergualin; rapamycin; T-cell receptor (Cohen et al., U.S. Pat. No. 5,114,721); T-cell receptor fragments (Offner et al., Science, 251: 430-432 (1991); WO 90/11294; Ianeway, Nature, 341: 482 (1989); and WO 91/01133); BAFF antagonists such as anti-BAFF antibodies and anti-BR3 antibodies and zTNF4 antagonists (for review, see Mackay and Mackay, Trends Immunol., 23:113-5 (2002)); biologic agents that interfere with T cell helper signals, such as anti-CD40 receptor or anti-CD40 ligand (CD154), including blocking antibodies to CD40-CD40 ligand (e.g., Durie et al., Science, 261: 1328-30 (1993); Mohan et al., J. Immunol., 154: 1470-80 (1995)) and CTLA4-Ig (Finck et al., Science, 265: 1225-7 (1994)); and T-cell receptor antibodies (EP 340,109) such as T10B9. Some immunosuppressive agents herein are also DMARDs, such as MTX. Examples of preferred immunosuppressive agents herein include cyclophosphamide, chlorambucil, azathioprine, leflunomide, MMF, or MTX.

The term “cytokine” is a generic term for proteins released by one cell population that act on another cell as intercellular mediators. Examples of such cytokines are lymphokines, monokines; interleukins (ILs) such as IL-1, IL-1a, IL-1b, IL-2, IL-3, IL-4, IL-5, IL-6, IL-7, IL-8, IL-9, IL-1, IL-12, IL-15, including PROLEUKIN® rIL-2; a tumor necrosis factor such as TNF-α or TNF-β; and other polypeptide factors including LIF and kit ligand (KL). As used herein, the term cytokine includes proteins from natural sources or from recombinant cell culture and biologically active equivalents of the native-sequence cytokines, including synthetically produced small-molecule entities and pharmaceutically acceptable derivatives and salts thereof. A “cytokine antagonist” is a molecule that inhibits or antagonizes such cytokines by any mechanism, including, for example, antibodies to the cytokine, antibodies to the cytokine receptor, and immunoadhesins.

The term “integrin” refers to a receptor protein that allows cells both to bind and respond to the extracellular matrix and is involved in a variety of cellular functions such as wound healing, cell differentiation, homing of tumor cells and apoptosis. They are part of a large family of cell adhesion receptors that are involved in cell-extracellular matrix and cell-cell interactions. Functional integrins consist of two transmembrane glycoprotein subunits, called α and β, which are non-covalently bound. The α subunits all share some homology to each other, as do the β subunits. The receptors always contain one a chain and one β chain. Examples include α6β1, α3β1, α7β1, the α4 chain such as α4β1, the β7 chain such as the β7 integrin subunit of α4β7 and/or αEβ7, LFA-1 etc. As used herein, the term “integrin” includes proteins from natural sources or from recombinant cell culture and biologically active equivalents of the native-sequence integrin, including synthetically produced small-molecule entities and pharmaceutically acceptable derivatives and salts thereof.

An “integrin antagonist” is a molecule that inhibits or antagonizes such integrins by any mechanism, including, for example, antibodies to the integrin. Examples of “integrin antagonists or antibodies” herein include an LFA-1 antibody, such as efalizumab (RAPTIVA®) commercially available from Genentech, or other CD11/11a and CD18 antibodies, or an a 4 integrin antibody such as natalizumab (ANTEGREN®) available from Biogen-IDEC, or diazacyclic phenylalanine derivatives (WO 2003/89410), phenylalanine derivatives (WO 2003/70709, WO 2002/28830, WO 2002/16329 and WO 2003/53926), phenylpropionic acid derivatives (WO 2003/10135), enamine derivatives (WO 2001/79173), propanoic acid derivatives (WO 2000/37444), alkanoic acid derivatives (WO 2000/32575), substituted phenyl derivatives (U.S. Pat. Nos. 6,677,339 and 6,348,463), aromatic amine derivatives (U.S. Pat. No. 6,369,229), ADAM disintegrin domain polypeptides (US 2002/0042368), antibodies to αvβ3 integrin (EP 633945), anti-β7 antibodies such as rhuMAb β7 (US 2006/0093601) and MLN-02 (Millennium Pharmaceuticals), anti-a4 antibodies such as TYSABR1 ® (Biogen-IDEC-Elan), T0047 (GSK/Tanabe), CDP-323 (oral) (UCB), aza-bridged bicyclic amino acid derivatives (WO 2002/02556), etc.

For the purposes herein, “tumor necrosis factor alpha” or “TNF-α” refers to a human TNF-α molecule comprising the amino acid sequence as described in Pennica et al., Nature, 312:721 (1984) or Aggarwal et al., JBC, 260:2345 (1985). A “TNF-α inhibitor” herein is an agent that inhibits, to some extent, a biological function of TNF-α, generally through binding to TNF-α and neutralizing its activity. Examples of TNF-α inhibitors specifically contemplated herein are etanercept (ENBREL®), infliximab (REMICADE®), and adalimumab (HUMIRA™).

Examples of “disease-modifying anti-rheumatic drugs” or “DMARDs” include hydroxycloroquine, sulfasalazine, MTX, leflunomide, etanercept, infliximab (plus oral and subcutaneous MTX), azathioprine, D-penicillamine, gold salts (oral), gold salts (intramuscular), minocycline, cyclosporine including cyclosporine A and topical cyclosporine, staphylococcal protein A (Goodyear and Silverman, J. Exp. Med., 197(9): 1125-39 (2003)), including salts and derivatives thereof, etc. A preferred DMARD herein is MTX.

Examples of “non-steroidal anti-inflammatory drugs” or “NSAIDs” include aspirin, acetylsalicylic acid, ibuprofen, naproxen, indomethacin, sulindac, tolmetin, COX-2 inhibitors such as celecoxib (CELEBREX®; 4-(5-(4-methylphenyl)-3-(trifluoromethyl)-1H-pyrazol-1-yl)benzenesulfonamide and valdecoxib (BEXTRA®), and meloxicam (MOBIC®), including salts and derivatives thereof, etc. Preferably, they are aspirin, naproxen, ibuprofen, indomethacin, or tolmetin.

“Corticosteroid” refers to any one of several synthetic or naturally occurring substances with the general chemical structure of steroids that mimic or augment the effects of the naturally occurring corticosteroids. Examples of synthetic corticosteroids include prednisone, prednisolone (including methylprednisolone, such as SOLU-MEDROL® methylprednisolone sodium succinate), dexamethasone or dexamethasone triamcinolone, hydrocortisone, and betamethasone. The preferred corticosteroids herein are prednisone, methylprednisolone, hydrocortisone, or dexamethasone.

A “medicament” is an active drug to treat RA or joint damage or the signs or symptoms or side effects of RA or joint damage.

The term “pharmaceutical formulation” refers to a sterile preparation that is in such form as to permit the biological activity of the medicament to be effective, and which contains no additional components that are unacceptably toxic to a subject to which the formulation would be administered.

A “sterile” formulation is aseptic or free from all living microorganisms and their spores.

A “package insert” is used to refer to instructions customarily included in commercial packages of therapeutic products or medicaments, that contain information about the indications, usage, dosage, administration, contraindications, other therapeutic products to be combined with the packaged product, and/or warnings concerning the use of such therapeutic products or medicaments, etc.

A “kit” is any manufacture (e.g., a package or container) comprising at least one medicament for treatment of RA or joint damage. The manufacture is preferably promoted, distributed, or sold as a unit for performing the methods of the present invention.

A “target audience” is a group of people or an institution to whom or to which a particular medicament is being promoted or intended to be promoted, as by marketing or advertising, especially for particular uses, treatments, or indications, such as individual patients, patient populations, readers of newspapers, medical literature, and magazines, television or internet viewers, radio or internet listeners, physicians, drug companies, etc.

The word “label” when used herein refers to a compound or composition that is conjugated or fused directly or indirectly to a reagent such as a nucleic acid probe or an antibody and facilitates detection of the reagent to which it is conjugated or fused. The label may itself be detectable (e.g., radioisotope labels or fluorescent labels) or, in the case of an enzymatic label, may catalyze chemical alteration of a substrate compound or composition that is detectable. The term is intended to encompass direct labeling of a probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

### II. Modes for Carrying Out the Invention

The subject or patient herein has been previously administered rituximab to treat the RA and was not responsive to that rituximab treatment.

The present invention provides, in one embodiment, a method for treating such non-responsive RA patient with an anti-CD20 antibody other than rituximab that is selected from a particular group. This group consists of:
(1) ofatumumab (with sequences of one light-chain variable region and two alternative heavy-chain variable regions given below),
(2) veltuzumab (with sequences of one light-chain and one of two heavy-chain variable regions given below),
(3) a small, modular immunopharmaceutical (SMIP) (otherwise known as an immunopharmaceutical or TRU-015) (with sequence given below),
(4) one of three CD20-binding antibodies (otherwise known as AME 33, AME 133, and AME 133v) (with light- and heavy-chain variable region sequences given below for the first two antibodies and the full sequence for the third antibody), and
(5) a humanized type II anti-CD20 IgG1 antibody with bisected afucosylated carbohydrates in its Fc region (otherwise known as GA101) (with sequences of light- and heavy-chain variable regions given below).

In another aspect, the invention provides a method for treating joint damage in a subject that is not responsive to rituximab comprising administering to the subject one of the anti-CD20 antibodies above, wherein the amount of anti-CD20 antibody administered is effective in achieving a reduction in the joint damage.

Optionally in such method, at least about one month, preferably at least about two months, and more preferably at least about 52 weeks after the administration, the subject is tested for reduction in the joint damage. For example, the subject is given an imaging test (such as a radiographic test) that measures a reduction in the joint damage as compared to baseline prior to the administration, indicating that the subject has been successfully treated, which test preferably measures a total modified Sharp score.

In another preferred embodiment, the joint damage is caused by arthritis, preferably RA, and more preferably early or incipient RA. Preferably, in this method regarding the about 52-week assessment, a second medicament is administered in an effective amount, wherein the anti-CD20 antibody is a first medicament. In one aspect, the second medicament is more than one medicament. In another aspect, the second medicament is t one of those set forth above, including an immunosuppressive agent, a DMARD, a different antibody against CD20 than the first medicament, an integrin antagonist, a NSAID, a cytokine antagonist, a bisphosphonate, or a combination thereof, most preferably MTX.

In a further aspect, the treatment methods herein further comprise re-treating the patient or subject by providing an additional administration to the patient or subject of an anti-CD20 antibody herein in an amount effective to treat RA or achieve a continued or maintained reduction in joint damage as compared to the effect of a prior administration of the anti-CD20 antibody. Preferably, the re-treatment is started at least about 24 weeks after the first administration of the antibody. In another preferred embodiment, one or more further re-treatments are commenced, more preferably at least about 24 weeks after the second administration of the antibody. In one aspect of this embodiment, the anti-CD20 antibody is additionally administered to the subject after the first re-treatment even if there is no clinical improvement in the subject at the time of RA testing or another imaging testing after a prior administration. In a preferred aspect, however, RA or joint damage has been reduced after the second or subsequent re-treatment as compared to the extent of RA or joint damage after the first assessment after re-treatment such as imaging assessment.

### III. Description of Anti-CD20 Antibodies

### Ofatumumab (HUMAX-CD2 ™)

The polynucleotide encoding the light-chain variable region of ofatumumab has the following sequence:

(SEQ ID NO:1)

ATGGAAGCCC CAGCTCAGCT TCTCTTCCTC CTGCTACTCT
GGCTCCCAGA TACCACCGGA GAAATTGTGT TGACACAGTC
TCCAGCCACC CTGTCTTTGT CTCCAGGGGA AAGAGCCACC
CTCTCCTGCA GGGCCAGTCA GAGTGTTAGC AGCTACTTAG
CCTGGTACCA ACAGAAACCT GGCCAGGCTC CCAGGCTCCT
CATCTATGAT GCATCCAACA GGGCCACTGG CATCCCAGCC
AGGTTCAGTG GCAGTGGGTC TGGGACAGAC TTCACTCTCA
CCATCAGCAG CCTAGAGCCT GAAGATTTTG CAGTTTATTA
CTGTCAGCAG CGTAGCAACT GGCCGATCAC CTTCGGCCAA
GGGACACGAC TGGAGATTAA AC

The polypeptide representing the light-chain variable region of ofatumumab has the following sequence:

(SEQ ID NO:2)

Met Glu Ala Pro Ala Gln Leu Leu Phe Leu Leu Leu
Leu Trp Leu Pro Asp Thr Thr Gly Glu Ile Val Leu
Thr Gln Ser Pro Ala Thr Leu Ser Leu Ser Pro Gly
Glu Arg Ala Thr Leu Ser Cys Arg Ala Ser Gln Ser
Val Ser Ser Tyr Leu Ala Trp Tyr Gln Gln Lys Pro
Gly Gln Ala Pro Arg Leu Leu Ile Tyr Asp Ala Ser
Asn Arg Ala Thr Gly Ile Pro Ala Arg Phe Ser Gly
Ser Gly Ser Gly Thr Asp Phe Thr Leu Thr Ile Ser
Ser Leu Glu Pro Glu Asp Phe Ala Val Tyr Tyr Cys
Gln Gln Arg Ser Asn Trp Pro Ile Thr Phe Gly Gln
Gly Thr Arg Leu Glu Ile Lys

The polynucleotide encoding a first (and preferred) heavy-chain variable region of ofatumumab has the following sequence:

(SEQ ID NO:3)

ATGGAGTTGG GACTGAGCTG GATTTTCCTT TTGGCTATTT
TAAAAGGTGT CCAGTGTGAA GTGCAGCTGG TGGAGTCTGG
GGGAGGCTTG GTACAGCCTG GCAGGTCCCT GAGACTCTCC
TGTGCAGCCT CTGGATTCAC CTTTAATGAT TATGCCATGC
ACTGGGTCCG GCAAGCTCCA GGGAAGGGCC TGGAGTGGGT
CTCAACTATT AGTTGGAATA GTGGTTCCAT AGGCTATGCG
GACTCTGTGA AGGGCCGATT CACCATCTCC AGAGACAACG
CCAAGAAGTC CCTGTATCTG CAAATGAACA GTCTGAGAGC
TGAGGACACG GCCTTGTATT ACTGTGCAAA AGATATACAG
TACGGCAACT ACTACTACGG TATGGACGTC TGGGGCCAAG
GGACCACGGT CACCGTCTCC TCAG

The polypeptide representing a first (and preferred) heavy-chain variable region of ofatumumab has the following sequence:

(SEQ ID NO:4)

Met Glu Leu Gly Leu Ser Trp Ile Phe Leu Leu Ala
Ile Leu Lys Gly Val Gln Cys Glu Val Gln Leu Val
Glu Ser Gly Gly Gly Leu Val Gln Pro Gly Arg Ser
Leu Arg Leu Ser Cys Ala Ala Ser Gly Phe Thr Phe
Asn Asp Tyr Ala Met His Trp Val Arg Gln Ala Pro
Gly Lys Gly Leu Glu Trp Val Ser Thr Ile Ser Trp
Asn Ser Gly Ser Ile Gly Tyr Ala Asp Ser Val Lys
Gly Arg Phe Thr Ile Ser Arg Asp Asn Ala Lys Lys
Ser Leu Tyr Leu Gln Met Asn Ser Leu Arg Ala Glu
Asp Thr Ala Leu Tyr Tyr Cys Ala Lys Asp Ile Gln
Tyr Gly Asn Tyr Tyr Tyr Gly Met Asp Val Trp Gly
Gln Gly Thr Thr Val Thr Val Ser Ser

The polypeptide representing a second heavy-chain variable region of ofatumumab has the following sequence:

(SEQ ID NO:5)

Met Phe Leu Gly Leu Ser Trp Ile Phe Leu Leu Ala
Ile Leu Lys Gly Val Gln Cys Glu Val Gln Leu Val
Glu Ser Gly Gly Gly Leu Val Gln Pro Gly Arg Ser
Leu Arg Leu Ser Cys Ala Ala Ser Gly Phe Thr Phe
Asn Asp Tyr Ala Met His Trp Val Arg Gln Ala Pro
Gly Lys Gly Leu Glu Trp Val Ser Thr Ile Ser Trp
Asn Ser Gly Ser Ile Gly Tyr Ala Asp Ser Val Lys
Gly Arg Phe Thr Ile Ser Arg Asp Asn Ala Lys Lys
Ser Leu Tyr Leu Gln Met Asn Ser Leu Arg Ala Glu
Asp Thr Ala Leu Tyr Tyr Cys Ala Lys Asp Ile Gln
Tyr Gly Asn Tyr Tyr Tyr Gly Met Asp Val Trp Gly
Gln Gly Thr Thr Val Thr Val Ser Ser

See also the sequences for the variable regions of ofatumumab (HuMax-CD20™ (2F2)) that are set forth as SEQ ID NOS:1-4 of US 2004/0167319, the disclosure of which is specifically incorporated herein by reference. In particular, the first variable heavy-region sequences (as defined herein) are SEQ ID NOS:1 and 2 (nucleotide and amino acid, respectively) of that patent application and the variable light-region sequences are SEQ ID NOS:3 and 4 (nucleotide and amino acid, respectively). The amino acid sequences of the second heavy-chain variable region and the light-chain variable region are also depicted in FIG. 53 of US 2004/0167319 with their designated CDR regions.

### Veltuzumab:

The sequences for the polypeptides representing (1) the signal peptide for all variable regions, (2) the light-chain variable region, and (3) two alternative heavy-chain variable regions of veltuzumab are shown below:

### Signal Sequence for Light-Chain and Heavy-Chain Variable Regions:

| | |
|---|---|
| M G W S C I I L F L V A T A T G V H S | (SEQ ID NO:6) |

### Light-Chain Variable Region:

(SEQ ID NO:7)

D I Q L T Q S P S S L S A S V G D R V T M T C R A
S S S V S Y I H W F Q Q K P G K A P K P W I Y A T
S N L A S G V P V R F S G S G S G T D Y T F T I S
S L Q P E D I A T Y Y C Q Q W T S N P P T F G G G
T K L E I K

(underlined portions in sequential order are CDR1, CDR2, and CDR3, respectively)

### Heavy-Chain Variable Region 1:

(SEQ ID NO:8)

Q V Q L Q Q S G A E V K K P G S S V K V S C K A S
G Y T F T S Y N M H W V K Q A P G Q G L E W I G A
I Y P G N G D T S Y N Q K F K G K A T L T A D E S
T N T A Y M E L S S L R S E D T A F Y Y C A R S T
Y Y G G D W Y F D V W G Q G T T V T V S

(underlined portions in sequential order are CDR1, CDR2, and CDR3, respectively)

### Heavy-Chain Variable Region 2:

(SEQ ID NO:9)

Q V Q L Q Q S G A E V K K P G S S V K V S C K A S
G Y T F S S Y N M H W V R Q A P G Q G L E W M G A
I Y P G N G D T S Y N Q K F K G R A T I T A D E S
T N T A Y M E L S S L R S E D T A F Y F C A R S T
Y Y G G D W Y F D V W G Q G T T V T V S

(underlined portions in sequential order are CDR1, CDR2, and CDR3, respectively) See also FIGS. 5A, 5B, and 5C, respectively, of US 2003/0219433, the disclosure of which is specifically incorporated herein by reference.

### Immunopharmaceutical (TRU-015):

The polynucleotide encoding the polypeptide TRU-015 has the following sequence:

(SEQ ID NO:10)

AAGCTTGCCG CCATGGATTT TCAAGTGCAG ATTTTCAGCT
TCCTGCTAAT CAGTGCTTCA GTCATAATGT CCAGAGGACA
AATTGTTCTC TCCCAGTCTC CAGCAATCCT GTCTGCATCT
CCAGGGGAGA AGGTCACAAT GACTTGCAGG GCCAGCTCAA
GTGTAAGTTA CATGCACTGG TACCAGCAGA AGCCAGGATC
CTCCCCCAAA CCCTGGATTT ATGCCCCATC CAACCTGGCT
TCTGGAGTCC CTGCTCGCTT CAGTGGCAGT GGGTCTGGGA
CCTCTTACTC TCTCACAATC AGCAGAGTGG AGGCTGAAGA
TGCTGCCACT TATTACTGCC AGCAGTGGAG TTTTAACCCA
CCCACGTTCG GTGCTGGGAC CAAGCTGGAG CTGAAAGATG
GCGGTGGCTC GGGCGGTGGT GGATCTGGAG GAGGTGGGAG
CTCTCAGGCT TATCTACAGC AGTCTGGGGC TGAGTCGGTG
AGGCCTGGGG CCTCAGTGAA GATGTCCTGC AAGGCTTCTG
GCTACACATT TACCAGTTAC AATATGCACT GGGTAAAGCA
GACACCTAGA CAGGGCCTGG AATGGATTGG AGCTATTTAT
CCAGGAAATG GTGATACTTC CTACAATCAG AAGTTCAAGG
GCAAGGCCAC ACTGACTGTA GACAAATCCT CCAGCACAGC
CTACATGCAG CTCAGCAGCC TGACATCTGA AGACTCTGCG
GTCTATTTCT GTGCAAGAGT GGTGTACTAT AGTAACTCTT
ACTGGTACTT CGATGTCTGG GGCACAGGGA CCACGGTCAC
CGTCTCTGAT CAGGAGCCCA AATCTTGTGA CAAAACTCAC
ACATCTCCAC CGTGCTCAGC ACCTGAACTC CTGGGTGGAC
CGTCAGTCTT CCTCTTCCCC CCAAAACCCA AGGACACCCT
CATGATCTCC CGGACCCCTG AGGTCACATG CGTGGTGGTG
GACGTGAGCC ACGAAGACCC TGAGGTCAAG TTCAACTGGT
ACGTGGACGG CGTGGAGGTG CATAATGCCA AGACAAAGCC
GCGGGAGGAG CAGTACAACA GCACGTACCG TGTGGTCAGC
GTCCTCACCG TCCTGCACCA GGACTGGCTG AATGGCAAGG
AGTACAAGTG CAAGGTCTCC AACAAAGCCC TCCCAGCCCC
CATCGAGAAA ACCATCTCCA AAGCCAAAGG GCAGCCCCGA
GAACCACAGG TGTACACCCT GCCCCCATCC CGGGATGAGC
TGACCAAGAA CCAGGTCAGC CTGACCTGCC TGGTCAAAGG
CTTCTATCCA AGCGACATCG CCGTGGAGTG GGAGAGCAAT
GGGCAGCCGG AGAACAACTA CAAGACCACG CCTCCCGTGC
TGGACTCCGA CGGCTCCTTC TTCCTCTACA GCAAGCTCAC
CGTGGACAAG AGCAGGTGGC AGCAGGGGAA CGTCTTCTCA
TGCTCCGTGA TGCATGAGGC TCTGCACAAC CACTACACGC
AGAAGAGCCT CTCCCTGTCT CCGGGTAAAT GATCTAGA

The polypeptide TRU-015 has the following sequence:

(SEQ ID NO: 11)
Met Asp Phe Gln Val Gln Ile Phe Ser Phe Leu Leu Ile Ser Ala Ser Val Ile Met Ser Arg Gly
Gln Ile Val Leu Ser Gln Ser Pro Ala Ile Leu Ser Ala Ser Pro Gly Glu Lys Val Thr Met Thr
Cys Arg Ala Ser Ser Ser Val Ser Tyr Met His Trp Tyr Gln Gln Lys Pro Gly Ser Ser Pro Lys
Pro Trp Ile Tyr Ala Pro Ser Asn Leu Ala Ser Gly Val Pro Ala Arg Phe Ser Gly Ser Gly Ser
Gly Thr Ser Tyr Ser Leu Thr Ile Ser Arg Val Glu Ala Glu Asp Ala Ala Thr Tyr Tyr Cys Gln
Gln Trp Ser Phe Asn Pro Pro Thr Phe Gly Ala Gly Thr Lys Leu Glu Leu Lys Asp Gly Gly
Gly Ser Gly Gly Gly Gly Ser Gly Gly Gly Gly Ser Ser Gln Ala Tyr Leu Gln Gln Ser Gly Ala
Glu Ser Val Arg Pro Gly Ala Ser Val Lys Met Ser Cys Lys Ala Ser Gly Tyr Thr Phe Thr Ser
Tyr Asn Met His Trp Val Lys Gln Thr Pro Arg Gln Gly Leu Glu Trp Ile Gly Ala Ile Tyr Pro
Gly Asn Gly Asp Thr Ser Tyr Asn Gln Lys Phe Lys Gly Lys Ala Thr Leu Thr Val Asp Lys
Ser Ser Ser Thr Ala Tyr Met Gln Leu Ser Ser Leu Thr Ser Glu Asp Ser Ala Val Tyr Phe Cys
Ala Arg Val Val Tyr Tyr Ser Asn Ser Tyr Trp Tyr Phe Asp Val Trp Gly Thr Gly Thr Thr Val
Thr Val Ser Asp Gln Glu Pro Lys Ser Cys Asp Lys Thr His Thr Ser Pro Pro Cys Ser Ala Pro
Glu Leu Leu Gly Gly Pro Ser Val Phe Leu Phe Pro Pro Lys Pro Lys Asp Thr Leu Met Ile Ser
Arg Thr Pro Glu Val Thr Cys Val Val Val Asp Val Ser His Glu Asp Pro Glu Val Lys Phe Asn
Trp Tyr Val Asp Gly Val Glu Val His Asn Ala Lys Thr Lys Pro Arg Glu Glu Gln Tyr Asn Ser
Thr Tyr Arg Val Val Ser Val Leu Thr Val Leu His Gln Asp Trp Leu Asn Gly Lys Glu Tyr Lys
Cys Lys Val Ser Asn Lys Ala Leu Pro Ala Pro Ile Glu Lys Thr Ile Ser Lys Ala Lys Gly Gln Pro
Arg Glu Pro Gln Val Tyr Thr Leu Pro Pro Ser Arg Asp Glu Leu Thr Lys Asn Gln Val Ser Leu
Thr Cys Leu Val Lys Gly Phe Tyr Pro Ser Asp Ile Ala Val Glu Trp Glu Ser Asn Gly Gln Pro
Glu Asn Asn Tyr Lys Thr Thr Pro Pro Val Leu Asp Ser Asp Gly Ser Phe Phe Leu Tyr Ser Lys
Leu Thr Val Asp Lys Ser Arg Trp Gln Gln Gly Asn Val Phe Ser Cys Ser Val Met
His Glu Ala Leu His Asn His Tyr Thr Gln Lys Ser Leu Ser Leu Ser Pro Gly Lys

See also SEQ ID NOS:3 and 4, respectively, of US 2007/0059306, the disclosure of which is specifically incorporated herein by reference, for the nucleotide and amino acid sequences, respectively, of TRU-015.

### CD20-Binding Antibodies:

### AME 33:

The polynucleotide encoding the light-chain variable region of the AME 33 antibody has the following sequence:

(SEQ ID NO: 12)

GAAATTGTGT TGACGCAGTC TCCAGGCACC CTGTCTTTGT
CTCCAGGGGA AAGAGCCACC CTCTCCTGCA GGGCCAGCTC
AAGTGTACCG TACATCCACT GGTACCAGCA GAAACCTGGC
CAGGCTCCCA GGCTCCTCAT CTATGCCACA TCCGCTCTGG
CTTCTGGCAT CCCAGACAGG TTCAGTGGCA GTGGGTCTGG
GACAGACTTC ACTCTCACCA TCAGCAGACT GGAGCCTGAA
GATTTTGCAG TGTATTACTG TCAGCAGTGG CTGAGTAACC
CACCCACTTT TGGCCAGGGG ACCAAGCTGG AGATCAAA

The polypeptide representing the light-chain variable region of the AME 33 antibody has the following sequence:

| | |
|---|---|
| Glu Ile Val Leu Thr Gln Ser Pro Gly Thr Leu Ser Leu Ser Pro Gly Glu Arg Ala | (SEQ ID NO: 13) |
| Thr Lou Ser Cys Arg Ala Ser Ser Ser Val Pro Tyr Ile His Trp Tyr Gln Gln Lys | |
| Pro Gly Gln Ala Pro Arg Leu Leu Ile Tyr Ala Thr Ser Ala Leu Ala Ser Gly Ile | |
| Pro Asp Arg Phe Ser Gly Ser Gly Ser Gly Thr Asp Phe Thr Leu Thr Ile Ser Arg | |
| Leu Glu Pro Glu Asp Phe Ala Val Tyr Tyr Cys Gln Gln Trp Leu Ser Asn Pro | |
| Pro Thr Phe Gly Gln Gly Thr Lys Leu Glu Ile Lys | |

The polynucleotide encoding the heavy-chain variable region of the AME 33 antibody has the following sequence:

(SEQ ID NO: 14)

GAGGTGCAGC TGGTGCAGTC TGGAGCAGAG GTGAAAAAGC
CCGGGGAGTC TCTGAAGATC TCCTGTAAGG GTTCTGGCCG
TACATTTACC AGTTACAATA TGCACTGGGT GCGCCAGATG
CCCGGGAAAG GCCTGGAGTG GATGGGGGCT ATTTATCCCT
TGACGGGTGA TACTTCCTAC AATCAGAAGT CGAAACTCCA
GGTCACCATC TCAGCCGACA AGTCCATCAG CACCGCCTAC
CTGCAGTGGA GCAGCCTGAA GGCCTCGGAC ACCGCCATGT
ATTACTGTGC GAGATCGACT TACGTGGGCG GTGACTGGCA
GTTCGATGTC TGGGGCAAGG GGACCACGGT CACCGTCTCC
TCA

The polypeptide representing the heavy-chain variable region of the AME 33 antibody has the following sequence:

| | |
|---|---|
| Glu Val Gln Leu Val Gln Ser Gly Ala Glu Val Lys Lys Pro Gly Glu Ser Leu Lys | (SEQ ID NO: 15) |
| Ile Ser Cys Lys Gly Ser Gly Arg Thr Phe Thr Ser Tyr Asn Met His Trp Val Arg | |
| Gln Met Pro Gly Lys Gly Leu Glu Trp Met Gly Ala Ile Tyr Pro Leu Thr Gly Asp | |
| Thr Ser Tyr Asn Gln Lys Ser Lys Leu Gln Val Thr Ile Ser Ala Asp Lys Ser Ile | |
| Ser Thr Ala Tyr Leu Gln Trp Ser Ser Leu Lys Ala Ser Asp Thr Ala Met Tyr Tyr | |
| Cys Ala Arg Ser Thr Tyr Val Gly Gly Asp Trp Gln Phe Asp Val Trp Gly Lys Gly | |
| Thr Thr Val Thr Val Ser Ser | |

See also FIGS. 2-3 as well as SEQ ID NOS:59-62 of US 2005/0025764 and US 2006/0251652, the disclosures of which are specifically incorporated herein by reference, for light- and heavy-chain variable region nucleotide and amino acid AME 33 sequences.

### AME 133:

The polynucleotide encoding the light-chain variable region of the AME 133 antibody has the following sequence:

| | |
|---|---|
| GAA ATT GTG TTG ACG CAG TCT CCA GGC ACC CTG TCT TTG TCT CCA GGG | (SEQ ID NO: 16) |
| GAA AGA GCC ACC CTC TCC TGC AGG GCC AGC TCA AGT GTA CCG TAC | |
| ATC CAC TGG TAC CAG CAG AAA CCT GGC CAG GCT CCC AGG CTC CTC | |
| ATC TAT GCC ACA TCC GCT CTG GCT TCT GGC ATC CCA GAC AGG TTC AGT | |
| GGC AGT GGG TCT GGG ACA GAC TTC ACT CTC ACC ATC AGC AGA CTG | |
| GAG CCT GAA GAT TTT GCA GTG TAT TAC TGT CAG CAG TGG CTG AGT | |
| AAC CCA CCC ACT TTT GGC CAG GGG ACC AAG CTG GAG ATC AAA CGA | |
| ACT GTG GCT GCA CCA TCT GTC TTC ATC TTC CCG CCA ICT GAT GAG CAG | |
| TTG AAA TCT GGA ACT GCC TCT GTT GTG TGC CTG CTG AAT AAC TTC TAT | |
| CCC AGA GAG GCC AAA GTA CAG TGG AAG GTG GAT AAC GCC CTC CAA | |
| TCG GGT AAC TCC CAG GAG AGT GTC ACA GAG CAG GAC AGC AAG GAC | |
| AGC ACC TAC AGC CTC AGC AGC ACC CTG ACG CTG AGC AAA GCA GAC | |
| TAC GAG AAA CAC AAA GTC TAC GCC TGC GAA GTC ACC CAT CAG GGC | |
| CTG AGC TCG CCC GTC ACA AAG AGC TTC AAC AGG GGA GAG TGT TAG | |

The polypeptide representing the light-chain variable region of the AME 133 antibody has the following sequence:

| | |
|---|---|
| Glu Ile Val Leu Thr Gln Ser Pro Gly Thr Leu Ser Leu Ser Pro Gly Glu Arg Ala | (SEQ ID NO: 17) |
| Thr Leu Ser Cys Arg Ala Ser Ser Ser Val Pro Tyr Ile His Trp Tyr Gln Gln Lys | |
| Pro Gly Gln Ala Pro Arg Leu Leu Ile Tyr Ala Thr Ser Ala Leu Ala Ser Gly Ile | |
| Pro Asp Arg Phe Ser Gly Ser Gly Ser Gly Thr Asp Phe Thr Leu Thr Ile Ser Arg | |
| Leu Glu Pro Glu Asp Phe Ala Val Tyr Tyr Cys Gln Gln Trp Leu Ser Asn Pro | |
| Pro Thr Phe Gly Gln Gly Thr Lys Leu Glu Ile Lys Arg Thr Val Ala Ala Pro Ser | |
| Val Phe Ile Phe Pro Pro Ser Asp Glu Gln Leu Lys Ser Gly Thr Ala Ser Val Val | |
| Cys Leu Leu Asn Asn Phe Tyr Pro Arg Glu Ala Lys Val Gln Trp Lys Val Asp | |
| Asn Ala Leu Gln Ser Gly Asn Ser Gln Glu Ser Val Thr Glu Gln Asp Ser Lys | |
| Asp Ser Thr Tyr Ser Leu Ser Ser Thr Leu Thr Leu Ser Lys Ala Asp Tyr Glu | |
| Lys His Lys Val Tyr Ala Cys Glu Val Thr His Gln Gly Leu Ser Ser Pro Val | |
| Thr Lys Ser Phe Asn Arg Gly Glu Cys | |

The polypeptide representing the heavy-chain variable region of the AME 133 antibody has the following sequence:

| | |
|---|---|
| Glu Val Gln Leu Val Gln Ser Gly Ala Glu Val Lys Lys Pro Gly Glu Ser Leu | (SEQ ID NO: 18) |
| Lys Ile Ser Cys Lys Gly Ser Gly Arg Thr Phe Thr Ser Tyr Asn Met His Trp Val | |
| Arg Gln Met Pro Gly Lys Gly Leu Glu Trp Met Gly Ala Ile Tyr Pro Leu Thr | |
| Gly Asp Thr Ser Tyr Asn Gln Lys Ser Lys Leu Gln Val Thr Ile Ser Ala Asp Lys | |
| Ser Ile Ser Thr Ala Tyr Leu Gln Trp Ser Ser Leu Lys Ala Ser Asp Thr Ala Met | |
| Tyr Tyr Cys Ala Arg Ser Thr Tyr Val Gly Gly Asp Trp Gln Phe Asp Val Trp | |
| Gly Lys Gly Thr Thr Val Thr Val Ser Ser | |

See also the nucleotide and amino acid sequences for the light-chain variable region of AME 133 set forth as SEQ ID NOS:197 and 198, respectively, in US 2005/0136044, the disclosure of which is specifically incorporated herein by reference.

### AME 133v:

The polypeptide representing AME 133v, a fusion protein prepared from the AME 133 Fab region fused to modified BChE variant L530, has the following sequence:

| | |
|---|---|
| Glu Val Gln Leu Val Gln Ser Gly Ala Glu Val Lys Lys Pro Gly Glu Ser Leu | (SEQ ID NO: 19) |
| Lys Ile Ser Cys Lys Gly Ser Gly Arg Thr Phe Thr Ser Tyr Asn Met His Trp Val | |
| Arg Gln Met Pro Gly Lys Gly Leu Glu Trp Met Gly Ala Ile Tyr Pro Leu Thr | |
| Gly Asp Thr Ser Tyr Asn Gln Lys Ser Lys Leu Gln Val Thr Ile Ser Ala Asp Lys | |
| Ser Ile Ser Thr Ala Tyr Leu Gln Trp Ser Ser Leu Lys Ala Ser Asp Thr Ala Met | |
| Tyr Tyr Cys Ala Arg Ser Thr Tyr Val Gly Gly Asp Trp Gln Phe Asp Val Trp | |
| Gly Lys Gly Thr Thr Val Thr Val Ser Ser Ala Ser Thr Lys Gly Pro Ser Val Phe | |
| Pro Leu Ala Pro Ser Ser Lys Ser Thr Ser Gly Gly Thr Ala Ala Leu Gly Cys Leu | |
| Val Lys Asp Tyr Phe Pro Glu Pro Val Thr Val Ser Trp Asn Ser Gly Ala Leu Thr | |
| Ser Gly Val His Thr Phe Pro Ala | |
| Val Leu Gln Ser Ser Gly Leu Tyr Ser Leu Ser Ser Val Val Thr Val Pro Ser Ser | |
| Ser Leu Gly Thr Gln Thr Tyr Ile Cys Asn Val Asn His Lys Pro Ser Asn Thr Lys | |
| Val Asp Lys Lys Ala Glu Pro Lys Ser Cys Asp Lys Thr His Thr Cys Pro Pro | |
| Cys Pro Lys Leu Glu Asp Asp Ile Ile Ile Ala Thr Lys Asn Gly Lys Val Arg Gly | |
| Met Asn Leu Thr Val Phe Gly Gly Thr Val Thr Ala Phe Leu Gly Ile Pro Tyr Ala | |
| Gln Pro Pro Leu Gly Arg Leu Arg Phe Lys Lys Pro Gln Ser Leu Thr Lys Trp | |
| Ser Asp Ile Trp Asn Ala Thr Lys Tyr Ala Asn Ser Cys Cys Gln Asn Ile Asp Gln | |
| Ser Phe Pro Gly Phe Phe Gly Ser Glu Met Trp Asn Pro Asn Thr Asp Leu Ser | |
| Glu Asp Cys Leu Tyr Leu Asn Val Trp Ile Pro Ala Pro Lys Pro Lys Asn Ala | |
| Thr Val Leu Ile Trp Ile Tyr Gly Gly Gly Phe Gln Thr Gly Thr Ser Ser Leu His | |
| Val Tyr Asp Gly Lys Phe | |
| Leu Ala Arg Val Glu Arg Val Ile Val Val Ser Met Asn Tyr Arg Val Gly Ala | |
| Leu Gly Phe Leu Ala Leu Pro Gly Asn Pro Glu Ala Pro Gly Asn Met Gly Leu | |
| Phe Asp Gln Gln Leu Ala Leu Gln Trp Val Gln Lys Asn Ile Ala Ala Phe Gly | |
| Gly Asn Pro Lys Ser Val Thr Leu Phe Gly Glu Ser Ala Gly Ala Ala Ser Val Ser | |
| Leu His Leu Leu Ser Pro Gly Ser His Ser Leu Phe Thr Arg Ala Ile Leu Gln Ser | |
| Gly Ser Ala Asn Ala Pro Trp Ala Val Thr Ser Leu Tyr Glu Ala Arg Asn Arg | |
| Thr Leu Asn Leu Ala Lys Leu Thr Gly Cys Ser Arg Glu Asn Glu Thr Glu Ile Ile | |
| Lys Cys Leu Arg Asn Lys Asp Pro Gln Glu Ile Leu Leu Asn Glu Ala Phe Val | |
| Val Pro Tyr Gly Thr Asn Leu Ser Val Asn Phe Gly Pro Thr Val Asp Gly Asp | |
| Phe Leu Thr Asp Met Pro Asp Ile Leu Leu Glu Leu Gly Gln Phe Lys Lys Thr | |
| Gln Ile Leu Val Gly Val Asn Lys Asp Glu Gly Thr Ala Phe Leu Ala Tyr Gly | |
| Ala Pro Gly Phe Ser Lys Asp Asn Asn Ser Ile Ile Thr Arg Lys Gln Phe Gln Glu | |
| Gly Leu Lys Ile | |
| Phe Phe Pro Gly Val Ser Glu Phe Gly Lys Glu Ser Ile Leu Phe His Tyr Thr Asp | |
| Trp Val Asp Asp Gln Arg Pro Glu Asn Tyr Arg Glu Ala Leu Gly Asp Val Val | |
| Gly Asp Tyr Asn Phe Ile Cys Pro Ala Leu Glu Phe Thr Lys Lys Phe Ser Glu | |
| Trp Gly Asn Asn Ala Phe Phe Tyr Tyr Phe Glu His Arg Ser Ser Lys Leu Pro | |
| Trp Pro Glu Trp Met Gly Val Met His Gly Tyr Glu Ile Glu Phe Val Phe Gly Leu | |
| Pro Leu Glu Arg Arg Asp Asn Tyr Tin Lys Ala Glu Glu Ile Leu Ser Arg Ser Ile | |
| Val Lys Arg Trp Ala Asn Phe Ala Lys Tyr Gly Asn Pro Asn Glu Thr Gln Asn | |
| Asn Ser Thr Ser Trp Pro Val Phe Lys Ser Thr Glu Gln Lys Tyr Leu Thr Leu | |
| Asn Thr Glu Ser Thr Arg Ile Met Thr Lys Leu Arg Ala Gln Gln Cys Arg Phe | |
| Trp Thr Ser Phe Phe Pro Lys Val | |

See also SEQ ID NO:202 and FIG. 19 from US 2005/0136044, the disclosure of which is specifically incorporated herein by reference.

### Humanized Type II Anti-CD20 IgG1 Antibody With a Glycoengineered Fc Region:

The polynucleotide encoding the light-chain variable region of the humanized type II anti-CD20 IgG1 antibody (GA101) has the following sequence:

| | |
|---|---|
| GATATCGTGA TGACCCAGAC TCCACTCTCC CTGCCCGTCA CCCCTGGAGA | (SEQ ID NO: 20) |
| GCCCGCCAGC ATTAGCTGCA GGTCTAGCAA GAGCCTCTTG CACAGCAATG | |
| GCATCACTTA TTTGTATTGG TACCTGCAAA AGCCAGGGCA GTCTCCACAG CTCCTGATT | |
| ATCAAATGTC CAACCTTGTC TCTGGCGTCC CTGACCGGTT CTCCGGATCC GGGTCAGGC | |
| CTGATTTCAC ACTGAAAATC AGCAGGGTGG AGGCTGAGGA TGTTGGAGTT | |
| TATTACTGCG CTCAGAATCT AGAACTTCCT | |
| TACACCTTCG GCGGAGGGAC CAAGGTGGAG ATCAAACGTA CGGTG | |

The polypeptide representing the light-chain variable region of the humanized type II anti-CD20 IgG1 antibody (GA101) has the following sequence:

(SEQ ID NO: 21)
Asp Ile Val Met Thr Gln Thr Pro Leu Ser Leu Pro Val Thr Pro Gly Glu Pro Ala Ser Ile Ser
Cys Arg Ser Ser Lys Ser Leu Leu His Ser Asn Gly Ile Thr Tyr Leu Tyr Trp Tyr Leu Gln Lys
Pro Gly Gln Ser Pro Gln Leu Leu Ile Tyr Gln Met Ser Asn Leu Val Ser Gly Val Pro Asp Arg
Phe Ser Gly Ser Gly Ser Gly Thr Asp Phe Thr Leu Lys Ile Ser Arg Val Glu Ala Glu Asp Val
Gly Val Tyr Tyr Cys Ala Gln Asn Leu Glu Leu Pro Tyr Thr Phe Gly Gly Gly Thr Lys Val
Glu Ile Lys Arg Thr Val

The polynucleotide encoding the heavy-chain variable region of the humanized type II anti-CD20 IgG1 antibody (GA101) has the following sequence:

(SEQ ID NO: 22)

CAGGTGCAAT TGGTGCAGTC TGGCGCTGAA GTTAAGAAGC
CTGGGAGTTC AGTGAAGGTC TCCTGCAAGG CTTCCGGATA
CGCCTTCAGC TATTCTTGGA TGAACTGGGT GCGGCAGGCC
CCTGGACAAG GGCTCGAGTG GATGGGACGG ATCTTTCCCG
GCGATGGGGA TACTGACTAC AATGGGAAAT TCAAGGGCAG
AGTCACAATT ACCGCCGACA AATCCACTAG CACAGCCTAT
ATGGAGCTGA GCAGCCTGAG ATCTGAGGAC ACGGCCGTGT
ATTACTGTGC AAGAAATGTC TTTGATGGTT ACTGGCTTGT
TTACTGGGGC CAGGGAACCC TGGTCACCGT CTCCTCA

The polypeptide representing the heavy-chain variable region of the humanized type II anti-CD20 IgG1 antibody (GA101) has the following sequence:

| | |
|---|---|
| Gln Val Gln Leu Val Gln Ser Gly Ala Glu Val Lys Lys Pro Gly Ser Ser Val Lys Val Ser | (SEQ D NO: 23) |
| Cys Lys Ala Ser Gly Tyr Ala Phe Ser Tyr Ser Trp Met Asn Trp Val Arg Gln Ala Pro Gly | |
| Gln Gly Leu Glu Trp Met Gly Arg Ile Phe Pro Gly Asp Gly Asp Thr Asp Tyr Asn Gly Lys | |
| Phe Lys Gly Arg Val Thr Ile Thr Ala Asp Lys Ser Thr Ser Thr Ala Tyr Met Glu Leu Ser | |
| Ser Leu Arg Ser Glu Asp Thr Ala Val Tyr Tyr Cys Ala Arg Asn Val Phe Asp Gly Tyr Trp | |
| Leu Val Tyr Trp Gly Gln Gly Thr Leu Val Thr Val Ser Ser | |

See also US 2005/0123546, the disclosure of which is specifically incorporated herein by reference, regarding BHH2-KV1-GE (GA101), which was humanized by grafting CDR sequences from murine B-ly1 on framework regions with fully human IgG1-kappa germline sequences. FIG. 7 of US 2005/0123546 lists a selection of predicted CDR regions of B-ly1. The sequence for the BHH2 component of GA101 (the heavy-chain variable region) is presented in Tables 2 and 3 as SEQ ID NOS:31 (nucleotide) and 32 (amino acid). The KV1 component (the light-chain variable region) is presented in Tables 2 and 3 as SEQ ID NOS:75 (nucleotide) and 76 (amino acid). The apparent variable heavy-chain and light-chain signal sequences are also set forth in these Tables as SEQ ID NOS:73 (variable heavy-chain, nucleotide), 74 (variable heavy-chain, amino acid), 77 (variable light-chain, nucleotide), and 76 (variable light-chain, amino acid).

The overall sequence information of BHH2-KV1-GE (GA101) is predicted as:

### Variable Light Region (Overall):

SEQ ID NOS:75 and 76, nucleotide and amino acid, respectively

### Variable Light Region (Predicted) CDR1:

SEQ ID NOS:8 and 18, nucleotide and amino acid, respectively

### Variable Light Region (Predicted) CDR2:

SEQ ID NOS:9 and 19, nucleotide and amino acid, respectively

### Variable Light Region (Predicted) CDR3:

SEQ ID NOS:10 and 20, nucleotide and amino acid, respectively

### Variable Heavy Region (Overall):

SEQ ID NOS:31 and 32, nucleotide and amino acid, respectively

### Variable Heavy Region (Predicted) CDR 1:

Any one of SEQ ID NOS:5-7 and 15-17, nucleotide and amino acid, respectively

### Variable Heavy Region (Predicted) CDR2:

Any one of SEQ ID NOS:21-23 and 25-27, nucleotide and amino acid, respectively

### Variable Heavy Region (Predicted) CDR3:

SEQ ID NOS:24 and 28, nucleotide and amino acid, respectively

### Summary of Anti-CD20 Antibodies and Specific Embodiments Thereof:

Thus, in summary, the anti-CD20 antibody that can be used in the treatment methods herein are: (1) ofatumumab comprising the variable light amino acid sequence in SEQ ID NO:2 and the variable heavy amino acid sequence in SEQ ID NO:4 or in SEQ ID NO:5; (2) veltuzumab comprising the variable light amino acid sequence in SEQ ID NO:7 and the variable heavy amino acid sequence in SEQ ID NO:8 or in SEQ ID NO:9; (3) an immunopharmaceutical comprising SEQ ID NO:11; (4) a CD20-binding antibody comprising the variable light amino acid sequence in SEQ ID NO:13 and the variable heavy amino acid sequence in SEQ ID NO:15, or comprising the variable light amino acid sequence in SEQ ID NO:17 and the variable heavy amino acid sequence in SEQ ID NO:18, or comprising SEQ ID NO:19; or (5) a humanized type II anti-CD20 IgG1 antibody with bisected afucosylated carbohydrates in its Fc region and comprising the variable light amino acid sequence in SEQ ID NO:21 and the variable heavy amino acid sequence in SEQ ID NO:23.

In one embodiment, the anti-CD20 antibody is ofatumumab. Most preferably, the ofatumumab comprises the variable heavy amino acid sequence in SEQ ID NO:4. However, in another embodiment it comprises the variable heavy amino acid sequence in SEQ ID NO:5.

In another embodiment, the anti-CD20 antibody is veltuzumab. In one particular aspect, the veltuzumab comprises the variable heavy amino acid sequence in SEQ ID NO:8. In another specific aspect, the veltuzumab comprises the variable heavy amino acid sequence in SEQ ID NO:9.

In a further embodiment, the anti-CD20 antibody is the immunopharmaceutical (equivalent to TRU-015).

In a still further embodiment, the anti-CD20 antibody is the CD20-binding antibody comprising SEQ ID NOS:13 and 15 or 17 and 18, or SEQ ID NO:19. In a first aspect, the CD20-binding antibody comprises the variable light amino acid sequence in SEQ ID NO:13 and the variable heavy amino acid sequence in SEQ ID NO:15 (AME 33).

In another aspect, the CD20-binding antibody comprises the variable light amino acid sequence in SEQ ID NO:17 and the variable heavy amino acid sequence in SEQ ID NO:18 (AME 133). In a further aspect, the CD20-binding antibody comprises SEQ ID NO:19 (AME 133v).

In a fifth overall embodiment, the anti-CD20 antibody is the humanized type II anti-CD20 IgG1 antibody.

These anti-CD20 antibodies with which the patient or subject may be treated may be produced using any suitable method, including those described below and in the examples herein.

### IV. Production of Anti-CD20 Antibodies

The anti-CD20 antibodies herein are generally manufactured as follows.

### Ofatumumab (HUMAX-CD20™)

Ofatumumab (2F2) may be prepared, for example, in accordance with the procedures described in US 2004/0167319, the disclosure of which is specifically incorporated herein by reference. The first variable heavy-region sequence given herein for ofatumumab is depicted in SEQ ID NOS:1 and 2 (nucleotide and amino acid, respectively) and the variable light-region sequence given herein for ofatumumab is depicted in SEQ ID NOS:3 and 4 (nucleotide and amino acid, respectively) of this patent application. The amino acid sequences of the second heavy-chain variable region and the light-chain variable region are also depicted in FIG. 53 of US 2004/0167319 with their designated CDR regions.

Examples 1-3 of US 2004/0167319 disclose the specifics of preparation of 2F2. Specifically, fully human monoclonal antibodies to CD20 were prepared using HCo7 and KM mice that express human antibody genes.

In the KM mouse strain, the endogenous mouse kappa light chain gene has been homozygously disrupted as described in Chen et al., EMBO J., 12:811-820 (1993) and the endogenous mouse heavy chain gene has been homozygously disrupted as described in Example 1 of WO 2001/09187. This mouse strain carries a human kappa light chain transgene, KCo5, as described in Fishwild et al., Nature Biotechnology, 14:845-851 (1996). This mouse strain also carries a human heavy chain transchromosome composed of chromosome 14 fragment hCF (SC20) as described in WO 2002/43478.

The HCo7 mice have a JKD disruption in their endogenous light chain (kappa) genes (as described in Chen et al., supra), a CMD disruption in their endogenous heavy chain genes (as described in Example 1 of WO 2001/14424), a KCo5 human kappa light chain transgene (as described in Fishwild et al., supra), and a HCo7 human heavy chain transgene (as described in U.S. Pat. No. 5,770,429).

HCo7 and KM mice were immunized with human CD20 transfected NS/0 cells. For the first immunization, per mouse, 1×10⁷ cells in 150 μl PBS were mixed 1:1 with Complete Freund's Adjuvant and injected intra-peritoneally (i.p.). Subsequent i.p. immunizations were done using a similar amount of cells without adjuvant. Three and two days prior to fusion the mice were intravenously boosted with 0.5×10⁷ cells suspended in phosphate-buffered saline (PBS).

The presence of antibodies directed against human CD20 in the serum of the mice was monitored by flow cytometry using FACS analysis, using human CD20 transfected NS/0 cells as well as CD20 negative parental NS/0 cells.

The mouse splenocytes were isolated from the HCo7 and KM mice and fused with PEG to a mouse myeloma cell line based upon standard protocols. The resulting hybridomas were then screened for human IgGκ production by ELISA and for CD20 specificity using human CD20 transfected NS/O and SKBR3 cells by FACS analysis. Single cell suspensions of splenic lymphocytes from immunized mice were fused to one-fourth the number of SP2/0 nonsecreting mouse myeloma cells (ATCC, CRL 1581) with 50% PEG (Sigma). Cells were plated at approximately 1×10⁵/well in flat bottom microtiter plate, followed by about two week incubation in selective medium containing 10% fetal bovine serum, 10% P388D1 (ATCC, CRL TIB-63) conditioned medium, 3-5% origin (IGEN) in DMEM (Mediatech, CRL 10013, with high glucose, L-glutamine and sodium pyruvate) plus 5 mM HEPES, 0.055 mM 2-mercaptoethanol, 50 mg/mil gentamycin and 1×HAT (Sigma, CRL P-7185). After 1-2 weeks, cells were cultured in medium in which the HAT was replaced with HT. Individual wells were then screened by flow cytometry for human anti-CD20 monoclonal IgG antibodies. Once extensive hybridoma growth occurred, medium was monitored usually after 10-14 days. The antibody-secreting hybridomas were replated, screened again and, if still positive for human IgG, anti-CD20 monoclonal antibodies were subcloned by limiting dilution. The stable subclones were then cultured in vitro to generate small amounts of antibody in tissue culture medium for characterization. One clone was chosen from each hybridoma, which retained the reactivity of parent cells (by FACS). 5-10 vial cell banks were generated for each clone and stored in liquid nitrogen.

The isotype of the antibodies was determined by performing an isotype ELISA. Wells of microtiter plates were coated with 1 μg/ml of mouse anti-human kappa light chain, 50 μl/well in PBS incubated 4° C. overnight. After blocking with 5% chicken serum, the plates were reacted with supernatant and purified isotype control. Plates were then incubated at ambient temperature for 1-2 hours. The wells were then reacted with human IgG1, IgG2, IgG3 or IgG4-specific horseradish peroxidase-conjugated probes. Plates were developed and analyzed as described above.

One of the hybridoma cell lines generated expressed 2F2, a human monoclonal IgG1,κ antibody with the nucleotide sequences SEQ ID NOS:1 and 3 and the amino acid sequences SEQ ID NOS:2 and 4 of US 2004/0167319.

The V_{L} and V_{H} regions were then sequenced using primers and oligonucleotides in polymerase chain reactions (PCR) as disclosed in US 2004/0167319.

After the PCR products were analyzed on an agarose gel, the products were purified with the QIAEX II Gel Extraction Kit (Qiagen, Westburg, Leusden, Netherlands). Two independently amplified PCR products of each V_{H} and V_{L} region were cloned in pGEMT-Vector System II (Promega) according to manufacturer's protocol (1999, version 6).

After transformation to E. coli JM109, individual colonies were screened by colony PCR using T7 and SP6 primers, 30 annealing cycles at 55° C. Plasmid DNA from colonies was purified using the QIAPREP SPIN MINIPREP™ kit (Qiagen). The V_{H} and V_{L} regions were further analyzed by digesting a Nco1/Not1 (NE Biolabs, Westburg, Leusden, Netherlands) and analyzed on agarose gel.

The V-regions regions were sequenced after cloning in the pGEMT-Vector System II. Sequencing was performed at Baseclear (Leiden, Netherlands). The sequences were analyzed by aligning germline V-gene sequences in Vbase (www.mrc-cpe.cam.ac.uk/imt-doc/public/intro.htm).http://www.mrc-cpe.cam.ac.uk/vbase-ok.php?menu=901.

The heavy-chain and light-chain variable regions of the 2F2 antibody were amplified, using PCR, from a standard cloning vector, pGem-5Zf (Promega), using primers that included an optimal Kozak sequence and suitable restriction sites to clone the fragments in the GS constant region vectors pCONγ1f and PCONκ (Lonza).

After amplification, the fragments were purified and digested with the restriction enzymes for cloning and ligated in the two vectors. The heavy chain variable fragment was digested with HindIII and BsiWI and ligated into the pCONγ1f vector, which had been digested with HindIII and BsiWI, and dephosphorylated with alkaline phosphatase. The light-chain variable fragment was digested with HindIII and ApaI and ligated into the PCONκ vector, which had been digested with HindIII and ApaI, and dephosphorylated with alkaline phosphatase. Transformed E. coli colonies were checked by colony PCR, and two positive colonies of each heavy-chain (HC) and light-chain (LC) construct were grown for plasmid isolation. Isolated plasmid of these four clones was sequenced to confirm the sequence. Both of the HC clones and one of the LC clones were found to have the correct sequences.

The two HC and one LC constructs were combined to give two combinations of LC-HC and transiently co-transfected in CHO-KL cells to check the constructs for proper production of 2F2 antibody. Normal production levels were reached for all combinations in this expression experiment and one clone of each of the HC and LC constructs was chosen for construction of a double-gene vector.

Standard cloning procedures were used to combine the HC and LC constructs in a double-gene cloning vector, designated pCONγ1f/κ 2F2, by ligating the complete expression cassette from the heavy chain vector, pCONγ1f/variable-heavy, into the light chain vector, pCONκ/variable-light.

This construct was again functionally tested in a transient transfection in CHO-K1 cells and showed normal expression levels. The variable regions of the pCONγ1f/κ2F2 plasmid were sequenced to reconfirm the correct sequences.

Linear plasmid was prepared for stable transfections by digesting pCONγ1f/2F2 with a unique restriction enzyme, PvuI, cutting outside regions vital for expression. Complete linearization was confirmed by agarose gel electrophoresis and the DNA was purified and stored at −20° C. until use.

Six transfections of NS/0 host cells were performed, by electroporation with plasmid DNA, using the above linear DNA plasmid. Following transfection, the cells were distributed into 96-wells plates and incubated. S elective medium (containing 10% dialyzed fetal calf serum (dFCS) and 10 μM of the GS-inhibitor L-methionine sulphoximine but lacking glutamine) was added and the plates were monitored to determine when the non-transfected cells died to leave foci of transfected cells. For details concerning GS vector systems, see WO 1987/04462. The transfected plates were incubated for approximately three weeks to allow colony formation. The resulting colonies were examined microscopically to verify that the colonies were of a suitable size for assay (covering greater than 60% of the bottom of the well), and that only one colony was present in each well. Cell supernatants from 436 transfectants were screened for assembled antibody by IgG-K-ELISA.

Using this data, 111 transfectants were selected for progression and further assessment in static culture. Cultures of the selected cell lines were expanded and adapted to low-serum-containing medium (containing bovine serum albumin (BSA) and added 1% dFCS) and a further assessment of productivity in static culture was undertaken (ELISA and measurement of percentage confluence). The 65 highest ranking cell lines were selected for progression. A preliminary assessment of the productivity of the selected cell lines was made in batch shake flask suspension culture in low serum-containing medium (containing BSA and added 1% dFCS). Based upon harvest antibody concentration (by ELISA) and acceptable growth characteristics, 30 cell lines were selected for further evaluation in serum-free medium using a batch shake flask suspension culture.

The ten cell lines that produced the highest antibody concentrations were further evaluated in duplicate fed-batch shake flask suspension cultures in serum-free medium. Product concentrations at harvest were determined by protein A high performance liquid chromatography (HPLC), according to well-known standard methods.

IMMU-106 (hA20 or Veltuzumab)

FIG. 5 of US 2003/0219433 discloses the nucleotide sequences of hA20 light chain V genes, (hA20Vk) (FIG. 5A), and heavy chain V genes, hA20VH1 (FIG. 5B) and hA20VH2 (FIG. 5C), as well as the adjacent flanking sequences of the VKpBR2 (FIG. 5A) and VHpBS2 (FIGS. 5B and 5C) staging vectors, respectively. The non-translated nucleotide sequences are shown in lower-case letters. The restriction sites used for subcloning are underlined and indicated. The secretion signal peptide sequence is indicated by a double underline. Amino acid sequences are given as single-letter codes below the corresponding nucleotide sequence. The Kabat numbering scheme was used for amino acid residues. Amino acid residues numbered by a letter represent the insertion residue according to Kabat, and have the same number as that of the previous residue.

Methods for constructing veltuzumab are described, for example, in US 2003/0219433, the disclosure of which is specifically incorporated herein by reference. In one particular preparation method, for example, the antibody is constructed and prepared as follows:

Each variable chain is optionally constructed in two parts, a 5′- and 3′-half, designated as “A” and “B” respectively. Each half is optionally produced by PCR amplification of a single-strand synthetic oligonucleotide template with two short flanking primers, using Taq polymerase. The amplified fragments may be first cloned into the pCR4 TA cloning vector from Invitrogen (Carlsbad, Calif.) and subjected to DNA sequencing. The templates and primer pairs are listed in US 2003/0219433. Assembly of the full-length Vκ light chain may be accomplished by restriction enzyme digestion of each fragment with the appropriate 5′- and 3′-enzymes and ligation into the VKpBR2 vector previously digested with PvuII and BclI (BclI-digested end is compatible with that of BglII). The resulting ligated product contains the A fragment ligated to the PvuII site, the B fragment ligated to the BclI site, and the A and B fragments joined together at the BstBI site. VKpBR2 is a modified staging vector of VKpBR (Leung et al., Hybridoma, 13:469 (1994)), into which a XbaI restriction site is introduced at 14 bases upstream of the translation initiation codon. Upon confirmation of a correct open reading frame by DNA sequencing, the intact chain is removed from VKpBR2 as a XbaI-to-BamHI fragment and ligated into the pdHL2 expression vector. The vector containing only the Vκ sequence is designated as hA20VκpdHL2. The vector pdHL2 contains the expression cassettes for both human IgG1 C1, C2, C3, and hinge regions and the human kappa chain Ck under the control of the IgH enhancer and MT₁ promoter, as well as a mouse dhfr gene, controlled by a weak SV40 promoter, as a marker for selection of transfectants and co-amplification of the trans-genes (Gillies et al., J. Immunol. Methods, 125:191 (1989) and Losman et al., Cancer, 80:2660 (1997)). By replacing the Vκ and VH segments of pdHL2, different chimeric or humanized Abs can be expressed.

For the construction of the heavy chain, the appropriate oligonucleotides are PCR-amplified by their respective primer pairs. The same construction method as done for Vκ is carried out for both types of V_{H}, with the following modifications: the 5′-end restriction site of the A fragment is PstI and the 3′-end restriction site of the B fragment is BstEII. These fragments are joined together upon ligation into the VHpBS2 vector at a common NciI site, resulting in full-length VH sequences (shown in FIGS. 1B and 2C of US 2003/0219433) and confirmed by DNA sequencing. VHpBS2 is a modified staging vector of VHpBS (Leung et al., Hybridoma, 13:469 (1994)), into which a XhoI restriction site is introduced at 16 bases upstream of the translation initiation codon. The assembled VH genes are subcloned as XhoI-BamHI restriction fragments into the expression vector containing the Vκ sequence, hA20Vκ pdHL2. Since the heavy-chain region of pdHL2 lacks a BamHI restriction site, this ligation requires use of the HNB linker (the sequence of which is set forth in US 2003/0219433) to provide a bridge between the BamHI site of the variable chain and the HindIII site present in the pdHL2 vector.

Transfection and expression of the hA20 antibodies is optionally carried out as follows. Approximately 30 μg of the expression vectors for hA20 are linearized by digestion with SalI and transfected into Sp2/0-Ag14 cells by electroporation (450V and 25 μF). The transfected cells are plated into 96-well plates for two days and then selected for drug resistance by adding MTX into the medium at a final concentration of 0.025 μM. MTX-resistant colonies are expected to emerge in the wells in 2-3 weeks. Supernatants from colonies surviving selection are screened for human antibody secretion by ELISA assay.

Briefly, 1001 supernatants are added into the wells of a microtiter plate precoated with a goat anti-human IgG (GAH-IgG), F(ab′)₂ fragment-specific antibody and incubated for one hour at room temperature. Unbound proteins are removed by washing three times with wash buffer (PBS containing 0.05% POLYSORBATE 20™ surfactant). Horse-radish peroxidase (HRP)-conjugated GAH-IgG, Fc fragment-specific antibody is added to the wells. Following an incubation of one hour, the plate is washed. The bound HRP-conjugated antibody is revealed by reading A490 nm after the addition of a substrate solution containing 4 mM o-phenylenediamine dihydrochloride (OPD) and 0.04% H₂O₂. Positive cell clones are expanded and hA20 antibodies are purified from the cell-culture supernatant by affinity chromatography on a Protein A column.

### Immunopharmaceutical

CD20-specific SMIPs are described generally in US 2003/133939, 2003/0118592, and 2005/0136049, the disclosures of which are specifically incorporated herein by reference. Production of an exemplary CD20-specific SMIP, TRU-015, is described, for example, in US 2007/0059306, the disclosure of which is specifically incorporated herein by reference, and below.

TRU-015 is a recombinant (murine/human) single-chain protein that binds to the CD20 antigen. The binding domain was based on a publicly available human anti-CD20 antibody sequence. The binding domain is connected to the effector domain, the CH2 and CH3 domains of human IgG1, through a modified CSS hinge region. TRU-015 exists as a dimer in solution and the dimer has a theoretical molecular weight of approximately 106,000 daltons. The nucleotide sequence encoding TRU-015 and the amino acid sequence of TRU-015 are respectively set out in SEQ ID NOS:10 and 11 set forth above.

Referring to the amino acid sequence set out in SEQ ID NO:11, TRU-015 comprises the 2e12 leader peptide cloning sequence from amino acids 1-23; the 2H7 murine anti-human CD20 light-chain variable region with a lysine to serine (VHL11S) amino acid substitution at residue 11 in the variable region, which is reflected at position 34; a linker beginning at residue 129, with the linker having an additional serine at the end to incorporate the SacI restriction site for cassette shuffling; the 2H7 murine anti-human CD20 heavy-chain variable region, which lacks a serine residue at the end of the heavy-chain region; a human IgG1 Fc domain, including a modified hinge region comprising a (CSS) sequence; and wild-type CH2 and CH3 domains.

TRU-015 may be cultured in a bioreactor using appropriate media and then purified using a series of chromatography and filtration steps, including, for example, a step employing a virus reduction filter. The material may then be concentrated and formulated with suitable excipients such as, for example, sodium phosphate (e.g., 20 mM) and sucrose (e.g., 240 mM) at an appropriate physiologically acceptable pH, for example, pH 6-7, more preferably 6.0. The composition may then be filtered before filling into vials, such as glass vials, at a concentration, for example, of 10 mg/mL. Each glass vial may contain, for example, 5 mL of TRU-015 (50 mg/vial).

### CD20-Binding Antibodies

The CD20-binding antibody AME 33 is prepared as described, for example, in US 2005/0025764 and US 2006/0251652, the disclosures of which are specifically incorporated herein by reference. The polynucleotide and amino acid sequences for the heavy- and light-chain variable regions of AME 33 are presented in both these applications as FIGS. 2-3 (SEQ ID NOS:59-62). The amino acid sequences for the light- and heavy-chain variable regions of AME 33 are respectively set forth above as SEQ ID NOS:13 and 15.

Example 1 of US 2005/0025764 describes the preparation of AME 33 in detail, including setting forth the CDR regions for each variable domain. The light- and heavy-chain variable regions for the CD20-binding molecule AME 33 may be combined with light- and heavy-chain constant regions and expressed as Fabs or full antibodies (e.g., IgG). For example, FIGS. 10 and 11 of US 2005/0025764 show the complete light and heavy chains for AME 33, which include the light- and heavy-chain constant regions, which are underlined in FIGS. 10A and 11A. Alternatively, AME 33 may contain the heavy-chain constant regions shown in those two figures except with an amino acid substitution in the Fc region. In particular, the heavy-chain constant region shown in FIG. 11 of that patent application may contain a D280H mutation or a K290S mutation (FIG. 11A shows positions 280 and 290 in bold, without the mutations). FIG. 11B shows a bold and underlined “GAC.”

In particular, FIG. 10A shows that the AME 33 complete light-chain amino acid sequence is as follows, with the constant region underlined:

(SEQ ID NO: 41)

EIVLTQSPGTLSLSPGERATLSCRASSSVPYIHWYQQKPGQAPRLLIYAT
SALASGIPDRFSGSGSGTDFTLTISRLEPEDFAVYYCQQWLSNPPTFGQG
TKLEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC

FIG. 11A shows that the AME 33 complete heavy-chain amino acid sequence is as follows, with the constant region underlined:

(SEQ ID NO: 42)

EVQLVQSGAEVKKPGESLKISCKGSGRTFTSYNMHWVRQMPGKGL
EWMGAIYPLTGDTSYNQKSKLQVTISADKSISTAYLQWSSLKASDTAMYY
CARSTYVGGDWQFDVWGKGTTVTVSSASTKGPSVEPLAPSSKSTSGGTAA
LGCLVKDYFPEPVTVSWNSGALTSGVHTFPAVLQSSGLYSLSSVVTVPSS
SLGTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKSL
SLSPGK

The CD20-binding antibody AME 133 is prepared as disclosed, for example, in US 2005/0136044, the disclosure of which is specifically incorporated herein by reference, including Example VII. The polynucleotide and amino acid sequences for the light-chain variable region of AME 133 are set forth as SEQ ID NOS:197 and 198, respectively, in US 2005/0136044. The amino acid sequence for the light-chain variable region of AME 133 is set forth above as SEQ ID NO:17. The amino acid sequence for the heavy-chain variable region of AME 133 is set forth above as SEQ ID NO:18.

The CD20-binding antibody AME 133v (LY2469298) is prepared, for example, as disclosed in US 2005/0136044, the disclosure of which is specifically incorporated herein by reference. The generation of this antibody is described, for example, on page 24, paragraph 175. It is a fusion protein prepared from the AME 133 Fab region, fused to a modified BCHE variant L530. The polynucleotide and amino acid sequences of AME 133v are shown in FIG. 19 of US 2005/0136044, and the polypeptide sequence is SEQ ID NO:202 of US 2005/0136044, and SEQ ID NO:19 herein.

Regarding AME 133 and AME 133v, antibody-enzyme fusion proteins incorporating optimized variant residues were identified by library screening and the feasibility of using optimized BChE in ADEPT with CPT-11 was confirmed by targeting CD20. CD20 is a useful target antigen to test the feasibility of using optimized BChE in ADEPT as the antigen is abundantly expressed on human B lymphoma lines (3.2×10⁵ molecules/cell) and does not undergo significant internalization upon antibody binding. AME133 is a humanized anti-CD20 with fully human germline framework regions, generated at Applied Molecular Evolution using directed evolution strategies, and the monovalent Fab binds to CD20 with extremely high affinity (1×10⁻⁹ M). An exemplary model fusion protein (anti-CD20-BChE.4-1 (SEQ ID NO:202) is composed of AME133 Fab fused at the C-terminal end of the CH1 heavy chain domain to the N-terminus of modified BCHE variant L530. The BChE variant was truncated at amino acid 530 to abrogate its normal assembly into tetramers. The monomeric version of the enzyme exhibits the equivalent activity of each subunit of the naturally occurring tetrameric form, with no loss of activity due to allosteric effects as described by Blong et al., Biochem. J., 327 (Pt 3): 747-57 (1997).

For AME 133v, non-adherent HEK 293 cells were adapted to suspension culture in serum-free low protein media (UltraCULTURE, BioWhittaker) and transiently transfected with the fusion protein AME 133v, producing yields of 2-4 mg/L. A two-step purification 6 procedure was developed using anion-exchange chromatography on SEPHAROSE Q™ medium followed by a hydrophobic-interaction chromatography step on phenyl SEPHAROSE™ medium. This resulted in product of >90% purity with one major contaminant band detected by SDS-PAGE.

Many procedures known in the art may be used to express the CD20-binding molecules herein. For example, the three CD20-binding antibodies can be expressed as Fabs or IgGs in mammalian or other (including bacterial, fungal and plant) expression systems using either a single-vector or double-vector system. For expression in a single vector, both heavy and light chains are manufactured or cloned within an expression cassette, which contains all required regulatory elements for expression. Expression using two vectors uses two expression cassettes in separate plasmids. The single plasmid or combined plasmids are generally transfected into a host cell line such as Chinese Hamster Ovary (CHO) cells or the retinal cell line PerC6, selected for, and then expanded and cultured to express the Fab or IgG proteins as is known to those skilled in the art.

A bacterial expression system is preferably for producing Fabs. An example is to insert and express Fabs within a M13 viral expression system. Fabs so expressed are secreted into the periplasmic space of the bacteria and may be released therefrom by various methods, including hypotonic shock and freeze-thaw procedures known in the art. Fabs are also optionally generated from intact IgG by proteolytic cleavage using a protease such as papain. The Fab portion of the cleavage product can be purified from the Fc portion of the cleavage product.

Fabs and antibodies can be purified using a wide number of chromatographic and specific adsorption techniques known in the art (see, e.g., Harlow et al., supra). For example, antibodies can be readily purified from cellular supernatants by specific binding using Protein A affinity chromatography followed by MONO ST cation-exchange chromatography.

Humanized Type II Anti-CD20 IgG1 Antibody with Glycoengineered Fc Region

The molecule GA101 is a humanized type II anti-CD20 IgG1 antibody. It is humanized by grafting CDR sequences from the murine monoclonal antibody B-ly1 onto framework regions with fully human IgG1-kappa germline sequences. Also, the Fc region-carbohydrates of this antibody are glycoengineered using GLYCOMAB™ technology described in WO 2004/065540 (the disclosure of which is specifically incorporated herein by reference), leading to bisected afucosylated Fc region-carbohydrates. GA101 is BHH2-KV1-GE, the preparation of which is described, for example, in US 2005/0123546, the disclosure of which is specifically incorporated herein by reference. See especially Example 2 thereof.

The sequences for the BHH2 component (the heavy-chain variable region) is presented in Tables 2 and 3 of US 2005/0123546 as SEQ ID NOS:31 (nucleotide) and 32 (amino acid). The KV1 component (the light-chain variable region) is presented in Tables 2 and 3 as SEQ ID NOS:75 (nucleotide) and 76 (amino acid). The molecule also contains a human IgG1 constant region.

For GA101 production the high-homology antibody acceptor framework search was performed by aligning the mouse B-Ly1 protein sequence to a collection of human germ-line sequences and selecting that human sequence that showed the highest sequence identity. The sequence VH1 . . . 10 from the VBase database was chosen as the heavy chain framework acceptor sequence, and the VK . . . 2 . . . 40 sequence was chosen to be the framework acceptor for the light chain. Onto these two acceptor frameworks, the three CDRs of the mouse heavy and light variable domains were grafted. Since the framework 4 region is not part of the variable region of the germ line V gene, the alignment for that position was done individually. The JH4 region was chosen for the heavy chain, and the JK4 region was chosen for the light chain. Molecular modeling of the designed immunoglobulin domain revealed one spot potentially requiring the murine amino acid residues instead of the human ones outside of the CDR. Reintroducing murine amino acid residues into the human framework would generate the so-called back mutations. For example, the human acceptor amino acid residue at Kabat position 27 was back mutated to a tyrosine residue. Humanized antibody variants were designed that either included or omitted the back mutations. The humanized antibody light chain did not require any back mutations. After having designed the protein sequences, DNA sequences encoding these proteins were synthesized as detailed below.

To avoid introducing back mutations at critical amino acid residue positions (critical to retain good antigen binding affinity or antibody functions) of the human acceptor framework, it was investigated whether either the whole framework region 1 (FR1), or framework regions 1 (FR1) and 2 (FR2) together, could be replaced by human antibody sequences already having donor residues, or functionally equivalent ones, at those important positions in the natural human germline sequence. For this purpose, the VH frameworks 1 and 2 of the mouse Bly1 sequence were aligned individually to human germ-line sequences. Here, highest sequence identity was not important, and was not used, for choosing acceptor frameworks, but instead matching of several critical residues was assumed to be more important. Those critical residues comprise residues 24, 71, and 94 (Kabat numbering), and also those residues at position 27, 28, and 30 (Kabat numbering), which lie outside of the CDR1 definition by Kabat, but often are involved in antigen binding. The IMGT sequence VH . . . 3 . . . 15 was chosen as a suitable one. After having designed the protein sequences, DNA sequences encoding these proteins were synthesized as detailed below. Using this approach no back mutations were required either for the light or heavy chain, to retain good levels of antigen binding.

After the amino acid sequence of the humanized antibody V region was designed, the DNA sequence was generated. The DNA sequence data of the individual framework regions was found in the databases for human germline sequences. The DNA sequence of the CDR regions was taken from the corresponding murine cDNA data. With these sequences, the whole DNA sequence was virtually assembled. Having this DNA sequence data, diagnostic restriction sites were introduced in the virtual sequence, by introducing silent mutations, creating recognition sites for restriction endonucleases. To obtain the physical DNA chain, gene synthesis was performed, wherein oligonucleotides are designed from the genes of interest, such that a series of oligonucleotides is derived from the coding strand, and one other series is from the non-coding strand. The 3′ and 5′ ends of each oligonucleotide (except the very first and last in the row) always show complementary sequences to two primers derived from the opposite strand. When putting these oligonucleotides into a reaction buffer suitable for any heat stable polymerase, and adding Mg²⁺, dNTPs and a DNA polymerase, each oligonucleotide is extended from its 3′ end. The newly formed 3′ end of one primer then anneals with the next primer of the opposite strand, and extending its sequence further under conditions suitable for template dependant DNA chain elongation. The final product was cloned into a conventional vector for propagation in E. coli.

Human heavy- and light-chain leader sequences (for secretion) were added upstream of the above variable region sequences and these were then joined upstream of human IgG1 kappa constant heavy and light chain sequences, respectively, using standard molecular biology techniques. The resulting fill antibody heavy and light chain DNA sequences were subcloned into mammalian expression vectors (one for the light chain and one for the heavy chain) under the control of the MPSV promoter and upstream of a synthetic polyA site, each vector carrying an EBV OriP sequence. Antibodies were produced by co-transfecting HEK293-EBNA with the mammalian antibody heavy and light chain expression vectors, harvesting the conditioned culture medium 5 to 7 days post-transfection, and purifying the secreted antibodies by Protein A affinity chromatography, followed by cation-exchange chromatography and a final size-exclusion chromatographic step to isolate pure monomeric IgG1 antibodies. The antibodies were formulated in a 25 mM potassium phosphate, 125 mM sodium chloride, 100 mM glycine solution of pH 6.7. Glycoengineered variants of the humanized antibody variants were produced by co-transfection of the antibody expression vectors together with a GnT-III glycosyltransferase expression vectors, or together with a GnT-III expression vector plus a Golgi mannosidase II expression vector. The oligosaccharides attached to the Fc region of the antibodies were analyzed by MALDI/TOF-MS.

For oligosaccharide release for antibodies in solution, between 40 and 50 μg of antibody were mixed with 2.5 mU of PNGaseF (Glyko, U.S.A.) in 2 mM TRIS buffer, pH 7.0 in a final volume of 25 microliters, and the mix was incubated for 3 hours at 37° C.

The enzymatic digests containing the released oligosaccharides were incubated for a further 3 hours at room temperature after the addition of acetic acid to a final concentration of 150 mM, and were subsequently passed through 0.6 ml of cation-exchange resin (AG50W-X8 resin, hydrogen form, 100-200 mesh, BioRad, Switzerland) packed into a micro-bio-spin chromatography column (BioRad, Switzerland) to remove cations and proteins. One microliter of the resulting sample was applied to a stainless steel target plate, and mixed on the plate with 1 μl of sDHB matrix. sDHB matrix was prepared by dissolving 2 mg of 2,5-dihydroxybenzoic acid plus 0.1 mg of 5-methoxysalicylic acid in 1 ml of ethanol/10 mM aqueous sodium chloride 1:1 (v/v). The samples were air dried, 0.2 μl ethanol was applied, and the samples were finally allowed to re-crystallize under air.

The MALDI-TOF mass spectrometer used to acquire the mass spectra was a Voyager Elite (Perspective Biosystems). The instrument was operated in the linear configuration, with an acceleration of 20 kV and 80 ns delay. External calibration using oligosaccharide standards was used for mass assignment of the ions. The spectra from 200 laser shots were summed to obtain the final spectrum.

The purified, monomeric humanized antibody variants were tested for binding to human CD20 on Raji B-cell lymphoma target cells using a flow cytometry-based assay.

Human NK cells were isolated from freshly isolated peripheral blood mononuclear cells (PBMC) applying a negative selection enriching for CD16- and CD56-positive cells (MACS system, Miltenyi Biotec GmbH, Bergisch Gladbach/Germany). The purity determined by CD56 expression was between 88-95%. Freshly isolated NK cells were incubated in PBS without calcium and magnesium ions (3×10⁵ cells/ml) for 20 minutes at 37° C. to remove NK cell-associated IgG. Cells were incubated at 10⁶ cells/ml at different concentrations of anti-CD20 antibody (0, 0.1, 0.3, 1, 3, 10 μg/ml) in PBS, 0.1% BSA. After several washes antibody binding was detected by incubating with 1:200 FITC-conjugated F(ab′)₂ goat anti-human, F(ab′)₂ specific IgG (Jackson ImmunoResearch, West Grove, Pa./USA) and anti-human CD56-PE (BD Biosciences, Allschwil/Switzerland). The anti-FcgammaRIIIA 3G8 F(ab′)₂ fragments (Ancell, Bayport, Minn./USA) were added at a concentration of 10 μg/ml to compete binding of antibody glycovariants (3 μg/ml). The fluorescence intensity referring to the bound antibody variants was determined for CD56-positive cells on a FACSCALIBUR™ machine (BD Biosciences, Allschwil/Switzerland). CHO cells were transfected by electroporation (280 V, 950 μF, 0.4 cm) with an expression vector coding for the FcgammaRIIIA-Val158 α-chain and the γ-chain. Transfectants were selected by addition of 6 μg/ml puromycin and stable clones were analyzed by FACS using 10 μl FITC-conjugated-anti-FcgammaRIII 3G8 monoclonal antibody (BD Biosciences, Allschwil/Switzerland) for 10⁶ cells. Binding of IgG1 to FcgammaRIIIA-Va 1158-expressing CHO cells was performed analogously to the NK cell binding.

Important properties of the humanized B-Ly1 antibody are that it is a type II anti-CD20 antibody as defined in Cragg and Glennie, Blood, 103(7):2738-2743 (2004). It therefore did not induce, upon binding to CD20, any significant resistance to non-ionic detergent extraction of CD20 from the surface of CD20+human cells, using the assay described for this purposes in Polyak and Deans, Blood, 99(9):3256-3262 (2002). According to US 2005/0123546, the humanized B-Ly1 antibody induced less resistance to non-ionic detergent extraction of CD20 than the C2B8 antibody (another anti-CD20 antibody with identical sequence to rituximab (see US 2003/0003097, Reff). As expected of a type II anti-CD20 antibody, the humanized B-Ly1 did not have any significant complement-mediated lysis activity. The humanized B-Ly1 antibody was very potent in the homotypic aggregation assay. In this assay CD20-positive human cells, Daudi cells, were incubated in cell culture medium for up to 24 hours at 37° C. in a 5% CO₂ atmosphere in a mammalian cell incubator, with the antibody at a concentration of 1 microgram per ml and in parallel at a concentration of 5 micrograms per ml. The aggregates were reported to be larger that those induced by addition of the C2B8 control antibody. In addition, and consistent with the antibody being anti-CD20 type II, the humanized B-Ly1 antibody was reported to induce higher levels of apoptosis when CD20-positive human cells were incubated therewith, relative to a control under identical conditions using the C2B8 chimeric IgG1 antibody.

Glycoengineered variants of the humanized antibodies were produced by co-expression of GnTIII glycosyltransferase, together with the antibody genes, in mammalian cells. This led to an increase in the fraction of non-fucosylated oligosaccharides attached to the Fc region of the antibodies, including bisected non-fucosylated oligosaccharides, as has been described in WO 2004/065540 (FIGS. 17-19). The glycoengineered antibodies had significantly higher levels of binding to human FcgammaRIII receptors and ADCC activity as well, relative to the non-glycoengineered antibody and relative to the C2B8 antibody. The humanized B-Ly1 antibody was also more potent at inducing human B-cell depletion in a whole blood assay than the control C2B8 antibody. This was true both for the non-glycoengineered B-Ly1 antibody and for the glycoengineered version of it. The glycoengineered antibody was approximately 1000-fold more potent than the C2B8 control anti-CD20 antibody in depleting B-cells in the whole blood assay.

### Constant Regions

The light- and heavy-chain variable regions for the anti-CD20 antibodies herein that are identified by variable region sequences only are combined with constant regions as disclosed in any of the above patent applications describing such antibodies and incorporated herein by reference. Typically, the constant region consists of light- and heavy-chain constant regions and the antibodies are intact antibodies with an Fc. These sequences are preferably linked to a leader sequence, with an example of a leader given in the text of US 2005/0025764. Depending on the particular antibody, any human constant region allotype chain may be employed. For example, AME 33 may contain the light- and heavy-chain constant regions shown in FIGS. 10 and 11 of US 2005/0025764 (underlined residues in SEQ ID NOS:41 and 42 herein) except with an amino acid substitution in the Fc region. In particular, the heavy-chain constant region shown in FIG. 11 of US 2005/0025764 may contain a D280H mutation or a K290S mutation (FIG. 11A shows positions 280 and 290 in bold, without the mutations). FIG. 11B of US 2005/0025764 shows a bold and underlined “GAC.” This “GAC” may be changed to “CAT” so as to encode the D280H mutation.

### V. Pharmaceutical Formulations

Therapeutic formulations of the antibodies used in accordance with the present invention are prepared for storage by mixing the antibody having the desired degree of purity with optional pharmaceutically acceptable carriers, excipients, or stabilizers in the form of lyophilized formulations or aqueous solutions. For general information concerning formulations, see, e.g., Gilman et al., (eds.) (1990), The Pharmacological Bases of Therapeutics, 8th Ed., Pergamon Press; A. Gennaro (ed.), Remington's Pharmaceutical Sciences, 18th Edition, (1990), Mack Publishing Co., Eastori, Pa.; Avis et al., (eds.) (1993) Pharmaceutical Dosage Forms: Parenteral Medications Dekker, New York; Lieberman et al., (eds.) (1990) Pharmaceutical Dosage Forms: Tablets Dekker, New York; and Lieberman et al., (eds.) (1990), Pharmaceutical Dosage Forms: Disperse Systems Dekker, New York, Kenneth A. Walters (ed.) (2002) Dermatological and Transdermal Formulations (Drugs and the Pharmaceutical Sciences), Vol 119, Marcel Dekker.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low-molecular-weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™, or polyethylene glycol (PEG).

Exemplary anti-CD20 antibody formulations are described in the patent applications cited above that describe the antibodies herein, including those cited in the background section herein, the disclosures of all of which are specifically incorporated by reference herein.

Lyophilized formulations adapted for subcutaneous administration are described, for example, in U.S. Pat. No. 6,267,958 (Andya et al.). Such lyophilized formulations may be reconstituted with a suitable diluent to a high protein concentration and the reconstituted formulation may be administered subcutaneously to the mammal to be treated herein.

Crystallized forms of the antibodies are also contemplated. See, for example, US 2002/0136719A1 (Shenoy et al.).

The formulation herein may also contain more than one active compound (a second medicament as defined above), preferably those with complementary activities that do not adversely affect each other. The type and effective amounts of such medicaments depend, for example, on the amount and type of anti-CD20 antibody present in the formulation, and clinical parameters of the subjects. The preferred such second medicaments are noted herein.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed, for example, in Remington's Pharmaceutical Sciences 16th edition, Osol, A. Ed. (1980).

Examples of sustained-release preparations applicable herein include semi-permeable matrices of solid hydrophobic polymers containing the anti-CD20 antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

### VI. Treatment with the Antibody

The anti-CD20 antibodies herein may be administered in any dose, provided it is effective to treat the patient. A physician having ordinary skill in the art can readily determine and prescribe the effective amount of the pharmaceutical composition required, depending on such factors as the particular anti-CD20 antibody employed, prior clinical experience published in the literature on the anti-CD20 antibody employed, the patient's characteristics and clinical history, the type and severity of RA and joint damage, other medicines being given, and any side effects predicted. For example, the physician could start with doses of an anti-CD20 antibody, employed in the pharmaceutical composition at levels lower than that required in order to achieve the desired therapeutic effect and gradually increase the dosage until the desired effect is achieved. The effectiveness of a given dose or treatment regimen of the anti-CD20 antibody can be determined, for example, by assessing signs and symptoms and/or assessing inhibition of structural damage or of radiographic progression in the patient using the standard RA measures of efficacy.

The dose may be by weight or a fixed dose, preferably a fixed dose regardless of weight. An example of a weighted dose is 375 mg/m² weekly×4. As a general proposition, the effective amount of the antibody administered parenterally per dose will be in the range of about 20 mg to about 5000 mg, by one or more dosages, which can be translated to a dose by weight. Preferably the total dose is between about 50 and 4000 mg, preferably about 75 and 3000 mg, more preferably about 100 and 2000 mg, more preferably about 100 and 1000 mg, more preferably about 150 and 1000 mg, more preferably about 200 and 1000 mg, including doses of about 200, 300, 400, 500, 600, 700, 800, 900, 1000 mg, and 2000 mg. These doses may be given as a single dose or as multiple doses, for example, two to four doses. Such doses may be done by infusions, for example. More preferably, an anti-CD20 antibody herein is administered at a dose of between about 200 and 1000 mg as a single dose or as two doses (preferably the doses are infusions). In a more preferred embodiment, the anti-CD20 antibody is administered at about 200 mg×1 or 2, 300 mg×1 or 2, 400 mg×1 or 2, 500 mg×1 or 2, 600 mg×1 or 2, 700 mg×1 or 2, 800 mg×1 or 2, 900 mg×1 or 2, or 1000 mg×1 or 2. If administered in two doses, the drug in one embodiment is given on days 1 and 15, preferably intravenously, at the start of treatment.

Preferably, the frequency of dosings, if given in a multidose form, is about two to four doses within a period of about one month, or about two to three doses administered within a period of about 2 to 3 weeks.

As noted above, however, these suggested amounts of antibody are subject to a great deal of therapeutic discretion. The key factor in selecting an appropriate dose and scheduling is the result obtained, as indicated above. For example, relatively higher doses may be needed initially for the treatment of ongoing and acute RA or joint damage. To obtain the most efficacious results, the antibody is administered as close to the first sign, diagnosis, appearance, or occurrence of the RA or joint damage as possible or during remissions of the RA or joint damage.

In all the methods herein, the RA is preferably early or incipient RA. The subject or patient herein may be rheumatoid factor (RF) or anti-CCP positive or negative. These autoantibodies are strongly correlated, but may represent distinct clinical subsets of RA. Preferably, the subject or patient is positive for one or both of these autoantibodies, most preferably positive for both.

In all the inventive methods set forth herein, the anti-CD20 antibody may be unconjugated, such as a naked antibody, or may be conjugated with another molecule for further effectiveness, such as, for example, to improve half-life.

In another embodiment of all the methods herein, the anti-CD20 antibody herein is the only medicament administered to the subject to treat the RA or joint damage.

In an alternative aspect, one may administer a second medicament, as noted above, with the antibodies herein. The combined administration includes co-administration, using separate formulations or a single pharmaceutical formulation, and consecutive administration in either order, wherein preferably there is a time period while both (or all) active agents simultaneously exert their biological activities.

The second medicament includes, for example, an immunosuppressive agent, an antibody against CD20 other than the first medicament (that is the anti-CD20 antibody being the first medicament), cytokine antagonist such as a cytokine antagonist, integrin antagonist (e.g., antibody), corticosteroid, or any combination thereof. The type of such second medicament depends on various factors, including the type of RA and/or joint damage, the severity of the RA and/or joint damage, the condition and age of the subject, the type and dose of first medicament employed, etc.

Examples of such additional medicaments include an immunosuppressive agent (such as mitoxantrone (NOVANTRONE®), MTX, cyclophosphamide, chlorambucil, leflunomide, and azathioprine), intravenous immunoglobulin (gamma globulin), lymphocyte-depleting therapy (e.g., mitoxantrone, cyclophosphamide, CAMPATH™ antibodies, anti-CD4, cladribine, rituximab, a 2H7 antibody, a polypeptide construct with at least two domains comprising a de-immunized, autoreactive antigen or its fragment that is specifically recognized by the Ig receptors of autoreactive B-cells (WO 2003/68822), total body irradiation, bone marrow transplantation), integrin antagonist or antibody (e.g., an LFA-1 antibody such as efalizumab/RAPTIVA® commercially available from Genentech, or an a 4 integrin antibody such as natalizumab/ANTEGREN® available from Biogen, or others as noted above), drugs that treat symptoms secondary or related to RA and/or joint damage such as those noted herein, steroids such as corticosteroid (e.g., prednisolone, methylprednisolone such as SOLU-MEDROL™ methylprednisolone sodium succinate for injection, prednisone such as low-dose prednisone, dexamethasone, or glucocorticoid, e.g., via joint injection, including systemic corticosteroid therapy), non-lymphocyte-depleting immunosuppressive therapy (e.g., MMF or cyclosporine), a TNF-α inhibitor such as an antibody to TNF-α, DMARD, NSAID, plasmapheresis or plasma exchange, trimethoprim-sulfamethoxazole (BACTRIM™, SEPTRA™), mycophenolate mofetil, H2-blockers or proton-pump inhibitors (during the use of potentially ulcerogenic immunosuppressive therapy), levothyroxine, cyclosporin A (e.g. SANDIMMUNE®), somatostatin analogue, a DMARD or NSAID, cytokine antagonist such as antibody, anti-metabolite, immunosuppressive agent, rehabilitative surgery, radioiodine, thyroidectomy, anti-IL-6 receptor antagonist/antibody (e.g., ACTEMRA™ (tocilizumab)), or another B-cell antagonist such as BR3-Fc, TACI-Ig, anti-BR3 antibody, anti-CD40 receptor or anti-CD40 ligand (CD154), agent blocking CD40-CD40 ligand, epratuzumab (anti-CD22 antibody), lumiliximab (anti-CD23 antibody), or an antibody directed against human CD20 other than rituximab or the anti-CD20 antibodies used herein, such as a 2H7 antibody.

Preferred such medicaments include gamma globulin, an integrin antagonist, anti-CD4, cladribine, trimethoprimsulfamethoxazole, an H2-blocker, a proton-pump inhibitor, cyclosporine, a TNF-α inhibitor, a DMARD, an NSAID (to treat, for example, musculoskeletal symptoms), levothyroxine, a cytokine antagonist (including cytokine-receptor antagonist), an anti-metabolite, an immunosuppressive agent such as MTX or a corticosteroid, a bisphosphonate, and another antagonist to a B-cell surface marker, such as, for example, a small molecule to CD20, a CD22 antibody, a BR3 antibody, lumiliximab (anti-CD23 antibody), BR3-Fc, or TACI-Ig.

The more preferred such medicaments are an immunosuppressive agent such as MTX or a corticosteroid, a DMARD, a different antibody against CD20 than the first medicament, an integrin antagonist, a NSAID, a cytokine antagonist, a bisphosphonate, or a combination thereof.

In one particularly preferred embodiment, the second medicament is a DMARD, which is preferably selected from the group consisting of auranofin, chloroquine, D-penicillamine, injectable gold, oral gold, hydroxychloroquine, sulfasalazine, myocrisin, and MTX.

In another such embodiment, the second medicament is a NSAID, which is preferably selected from the group consisting of: fenbufen, naprosyn, diclofenac, etodolac and indomethacin, aspirin and ibuprofen.

In a further such embodiment, the second medicament is an immunosuppressive agent, which is preferably selected from the group consisting of etanercept, infliximab, adalimumab, leflunomide, anakinra, azathioprine, MTX, and cyclophosphamide.

In other preferred embodiments, the second medicament is selected from the group consisting of anti-α4, etanercept, infliximab, etanercept, adalimumab, kinaret, efalizumab, OPG, RANK-Fc, anti-RANKL, pamidronate, alendronate, actonel, zolendronate, rituximab, a 2H7 antibody, clodronate, MTX, azulfidine, hydroxychloroquine, doxycycline, leflunomide, SSZ, prednisolone, interleukin-1 receptor antagonist, prednisone, and methylprednisolone.

In still preferred embodiments, the second medicament is selected from the group consisting of MTX, infliximab, a combination of infliximab with MTX, etanercept, a corticosteroid, cyclosporin A, azathioprine, auranofin, hydroxychloroquine (HCQ), a combination of prednisolone with MTX and SSZ, a combination of MTX with SSZ and HCQ, a combination of cyclophosphamide with azathioprine and HCQ, and a combination of adalimumab with MTX. If the second medicament is a corticosteroid, preferably it is prednisone, prednisolone, methylprednisolone, hydrocortisone, or dexamethasone. Also, preferably, the corticosteroid is administered in lower amounts than are used if the anti-CD20 antibody is not administered to a subject treated with a corticosteroid. Most preferably, the second medicament is MTX.

All these second medicaments may be used in combination with each other or by themselves with the first medicament, so that the expression “second medicament” as used herein does not mean it is the only medicament besides the first medicament, respectively. Thus, the second medicament need not be one medicament, but may constitute or comprise more than one such drug.

These second medicaments as set forth herein are generally used in the same dosages and with administration routes as used hereinbefore or about from 1 to 99% of the heretofore-employed dosages. If such second medicaments are used at all, preferably, they are used in lower amounts than if the first medicament were not present, especially in subsequent dosings beyond the initial dosing with the first medicament, so as to eliminate or reduce side effects caused thereby.

For the re-treatment methods described herein, where a second medicament is administered in an effective amount with an antibody exposure, it may be administered with any exposure, for example, only with one exposure, or with more than one exposure. In one embodiment, the second medicament is administered with the initial exposure. In another embodiment, the second medicament is administered with the initial and second exposures. In a still further embodiment, the second medicament is administered with all exposures. It is preferred that after the initial exposure, such as of steroid, the amount of such second medicament is reduced or eliminated so as to reduce the exposure of the subject to an agent with side effects such as prednisone, prednisolone, methylprednisolone, and cyclophosphamide.

The combined administration of a second medicament includes co-administration (concurrent administration), using separate formulations or a single pharmaceutical formulation, and consecutive administration in either order, wherein preferably there is a time period while both (or all) active agents (medicaments) simultaneously exert their biological activities.

The anti-CD20 antibody herein is administered by any suitable means, including parenteral, topical, intraperitoneal, intrapulmonary, intranasal, and/or intralesional administration. Parenteral infusions include intramuscular, intravenous (i.v.), intraarterial, intraperitoneal, or subcutaneous (s.c.) administration. Intrathecal administration is also contemplated (see, e.g., US 2002/0009444 (Grillo-Lopez) concerning intrathecal delivery of an anti-CD20 antibody). In addition, the anti-CD20 antibody may suitably be administered by pulse infusion, e.g., with declining doses of the anti-CD20 antibody. Preferably, the dosing is given by i.v. or s.c. Whether the administration is i.v. or s.c. will depend on many factors, including the type of anti-CD20 antibody employed, the clinical history of the patient, the particular dosing and scheduling, etc. In some cases it may be preferable to give the antibody by s.c. rather than i.v. administration.

If multiple exposures of the anti-CD20 antibody are provided, each exposure may be provided using the same or a different administration means. In one embodiment, each exposure is by i.v. administration. In another embodiment, each exposure is given by s.c. administration. In yet another embodiment, the exposures are given by both i.v. and s.c. administration.

In one embodiment, the anti-CD20 antibody is administered as a slow i.v. infusion rather than an i.v. push or bolus. For example, a steroid such as prednisolone or methylprednisolone (e.g., about 80-120 mg i.v., more specifically about 100 mg i.v.) is administered about 30 minutes prior to any infusion of the anti-CD20 antibody. The anti-CD20 antibody is, for example, infused through a dedicated line.

For the initial dose of a multi-dose exposure to the anti-CD20 antibody, or for the single dose if the exposure involves only one dose, such infusion is preferably commenced at a rate of about 50 mg/hour. This may be escalated, e.g., at a rate of about 50 mg/hour increments every about 30 minutes to a maximum of about 400 mg/hour. However, if the subject is experiencing an infusion-related reaction, the infusion rate is preferably reduced, e.g., to half the current rate, e.g., from 100 mg/hour to 50 mg/hour. Preferably, the infusion of such dose of anti-CD20 antibody (e.g., an about 1000-mg total dose) is completed at about 255 minutes (4 hours 15 min.). Optionally, the subjects receive a prophylactic treatment of acetaminophen/paracetamol (e.g., about 1 g) and diphenhydramine HCl (e.g., about 50 mg or equivalent dose of similar agent) by mouth about 30 to 60 minutes prior to the start of an infusion.

If more than one infusion (dose) of anti-CD20 antibody is given to achieve the total exposure, the second or subsequent anti-CD20 antibody infusions in this infusion embodiment are preferably commenced at a higher rate than the initial infusion, e.g., at about 100 mg/hour. This rate may be escalated, e.g., at a rate of about 100 mg/hour increments every about 30 minutes to a maximum of about 400 mg/hour. Subjects who experience an infusion-related reaction preferably have the infusion rate reduced to half that rate, e.g., from 100 mg/hour to 50 mg/hour. Preferably, the infusion of such second or subsequent dose of anti-CD20 antibody (e.g., an about 1000-mg total dose) is completed by about 195 minutes (3 hours 15 minutes).

Aside from administration of one of the anti-CD20 antibodies herein to the patient by traditional routes as noted above, the present invention includes administration by gene therapy. Such administration of nucleic acids encoding the antibody is encompassed by the expression “administering an effective amount of an antibody.” See, for example, WO 1996/07321 concerning the use of gene therapy to generate intracellular antibodies.

There are two major approaches to getting the nucleic acid (optionally contained in a vector) into the patient's cells; in vivo and ex vivo. For in vivo delivery the nucleic acid is injected directly into the patient, usually at the site where the antibody is required. For ex vivo treatment, the patient's cells are removed, the nucleic acid is introduced into these isolated cells and the modified cells are administered to the patient either directly or, for example, encapsulated within porous membranes which are implanted into the patient (see, e.g. U.S. Pat. Nos. 4,892,538 and 5,283,187). There are a variety of techniques available for introducing nucleic acids into viable cells. The techniques vary depending upon whether the nucleic acid is transferred into cultured cells in vitro or in vivo in the cells of the intended host. Techniques suitable for the transfer of nucleic acid into mammalian cells in F vitro include the use of liposomes, electroporation, microinjection, cell fusion, DEAE-dextran, the calcium phosphate precipitation method, etc. A commonly used vector for ex vivo delivery of the gene is a retrovirus.

The currently preferred in vivo nucleic acid transfer techniques include transfection with viral vectors (such as adenovirus, Herpes simplex I virus, or adeno-associated virus) and lipid-based systems (useful lipids for lipid-mediated transfer of the gene are DOTMA, DOPE and DC-Chol, for example). In some situations it is desirable to provide the nucleic acid source with an agent specific for the target cells, such as an antibody specific for a cell-surface membrane protein on the target cell, a ligand for a receptor on the target cell, etc. Where liposomes are employed, proteins that bind to a cell-surface membrane protein associated with endocytosis may be used for targeting and/or to facilitate uptake, e.g. capsid proteins or fragments thereof tropic for a particular cell type, antibodies for proteins that undergo internalization in cycling, and proteins that target intracellular localization and enhance intracellular half-life. The technique of receptor-mediated endocytosis is described, for example, by Wu et al., J. Biol. Chem., 262:4429-4432 (1987); and Wagner et al., Proc. Natl. Acad. Sci. USA, 87:3410-3414 (1990). Gene-marking and gene-therapy protocols are described, for example, in Anderson et al., Science, 256:808-813 (1992) and WO 1993/25673.

Once the patient population most responsive to treatment with the anti-CD20 antibody has been identified, treatment with the antibody herein, alone or in combination with other medicaments, results in an improvement in the RA or joint damage, including signs or symptoms thereof. For instance, such treatment may result in an improvement in ACR measurements relative to a patient treated with the second medicament only (e.g., an immunosuppressive agent such as MTX), and/or may result in an objective response (partial or complete, preferably complete) as measured by ACR. Moreover, treatment with the combination of an antibody herein and at least one second medicament(s) preferably results in an additive, more preferably synergistic (or greater than additive) therapeutic benefit to the patient. Preferably, in this combination method the timing between at least one administration of the second medicament and at least one administration of the antibody herein is about one month or less, more preferably, about two weeks or less.

For purposes of the methods herein, success of treatment is determined as set forth above. Clinical improvement is preferably determined by assessing the number of tender or swollen joints, conducting a global clinical assessment of the patient, assessing erythrocyte sedimentation rate, assessing the amount of C-reactive protein level, or using composite measures of disease activity (disease response) such as the DAS-28, ACR-20, -50, or -70 scores.

For the treatment methods herein, an optional step is included to test for the subject's response to treatment after the administration step to determine that the level of response is effective to treat the RA or joint damage. For example, a step is optionally included to test the imaging (radiographic and/or MRI) score after administration and compare it to baseline imaging results obtained before administration to determine if treatment is effective by measuring if, and by how much, it has been changed. This test may be repeated at various scheduled or unscheduled time intervals after the administration to determine maintenance of any partial or complete remission.

Alternatively, the methods herein comprise a step of testing the subject, before administration, to see if one or more biomarkers or symptoms are present for RA or joint damage. In another method, a step may be included to check the subject's clinical history, as detailed above, for example, to rule out infections or malignancy as causes, for example, primary causes, of the subject's condition, prior to administering the antibody to the subject. Preferably, the joint damage is primary (i.e., the leading disease), and is not secondary, such as secondary to infection or malignancy, whether solid or liquid tumors.

In a further embodiment of all the methods herein, the subject has never been previously administered one or more drug(s) other than rituximab, such as an anti-TNF-α inhibitor, e.g., an anti-TNF-α or anti-TNF-α receptor antibody, to treat RA, or an immunosuppressive agent(s) to treat the RA or joint damage, and/or has never been previously treated with an antagonist to a B-cell surface marker other than rituximab (e.g., never been previously treated with a 2H7 antibody or a CD22 or BR3 antibody).

In a specific embodiment of this aspect, the subject has never been previously treated with an integrin antagonist such as anti-a4 integrin antibody or co-stimulation modulator, an immunosuppressive agent, a cytokine antagonist, an anti-inflammatory agent such as a NSAID, a DMARD other than MTX, except for azathioprine and/or leflunomide, a cell-depleting therapy, including investigational agents (e.g., CAMPATH, anti-CD4, anti-CD5, anti-CD3, anti-CD19, anti-CD11a, anti-CD22, or BLys/BAFF), a live/attenuated vaccine within 28 days prior to baseline, or a corticosteroid such as an intra-articular or parenteral glucocorticoid within four weeks prior to baseline.

More preferably, the subject has never been treated with an immunosuppressive agent, cytokine antagonist, integrin antagonist, corticosteroid, analgesic, a DMARD, or a NSAID. Still more preferably, the subject has never been treated with an immunosuppressive agent, cytokine antagonist, integrin antagonist, corticosteroid, DMARD, or NSAID. Most preferably, in all the methods of the invention herein, the subject has not been previously treated with an immunosuppressive agent before the administration of a first dose of anti-CD20 antibody.

Alternatively, the subject or patient has been previously administered other drugs besides rituximab, and in one embodiment the subject or patient was not responsive to such drugs for treating the RA or joint damage. Such drugs (other than rituximab) to which the subject may be non-responsive include, for example, chemotherapeutic agents, immunosuppressive agents, cytokine antagonists, integrin antagonists, corticosteroids, analgesics, or antagonists to B-cell surface markers other than an antibody directed against CD20. Preferably, such antagonists to B-cell surface markers are not antibodies or immunoadhesins, and are, for example, small-molecule inhibitors, or anti-sense oligonucleotides, or antagonistic peptides. More particularly, the drugs to which the subject may be non-responsive include immunosuppressive agents such as MTX, DMARDs, or TNF-alpha inhibitors.

In particularly preferred embodiments of the above-identified methods, the subject has exhibited an inadequate response to one or more anti-TNF-α inhibitors, and/or MTX is administered to the subject along with the anti-CD20 antibody.

In another embodiment, the subject or patient is responsive to therapy other than rituximab for the RA or joint damage.

In another preferred aspect, the subject was administered MTX prior to the baseline or start of treatment. More preferably, the MTX was administered at a dose of about 10-25 mg/week. Also, preferably, the MTX was administered for at least about 12 weeks prior to the baseline, and still more preferably the MTX was administered at a stable dose the last four weeks prior to the baseline. In other embodiments, the MTX was administered perorally or parenterally.

In a still further aspect, other than being non-responsive to rituximab, the subject may have had a relapse with the RA or joint damage or suffered organ damage such as kidney damage before being treated in any of the methods above, including after an initial exposure or later exposure to an anti-CD20 antibody herein. However, preferably, the subject has not relapsed with the RA or joint damage with any drug other than rituximab and more preferably has not had such a relapse before at least the initial treatment.

In a further embodiment, the subject does not have a malignancy, including a B-cell malignancy, solid tumors, hematologic malignancies, or carcinoma in situ (except basal cell and squamous cell carcinoma of the skin that have been excised and cured).

In a still further embodiment, the subject does not have rheumatic autoimmune disease other than RA, or significant systemic involvement secondary to RA (including but not limited to vasculitis, pulmonary fibrosis, or Felty's syndrome). In another embodiment, the subject does not have functional class IV as defined by the ACR Classification of Functional Status in RA. In a further embodiment, the subject does not have inflammatory joint disease other than RA (including, but not limited to, gout, reactive arthritis, psoriatic arthritis, seronegative spondyloarthropathy, Lyme disease), or other systemic autoimmune disorder (including, but not limited to, SLE, inflammatory bowel disease, scleroderma, inflammatory myopathy, mixed connective tissue disease, or any overlap syndrome). In another embodiment, the subject does not have JIA, JRA, and/or RA before age 16. In another embodiment, the subject does not have significant and/or uncontrolled cardiac or pulmonary disease (including obstructive pulmonary disease), or significant concomitant disease, including but not limited to, nervous system, renal, hepatic, endocrine or gastrointestinal disorders., nor primary or secondary immunodeficiency (history of, or currently active), including known history of HIV infection. In another aspect, the subject does not have any neurological (congenital or acquired), vascular or systemic disorder which could affect any of the efficacy assessments, in particular, joint pain and swelling (e.g., Parkinson's disease, cerebral palsy, diabetic neuropathy). In a still further embodiment, the subject does not have MS. In a yet further embodiment, the subject does not have lupus or Sjögren's syndrome. In still another embodiment, the subject does not have an autoimmune disease other than RA. In yet another aspect of the invention, any joint damage in the subject is not associated with an autoimmune disease or with an autoimmune disease other than RA, or with a risk of developing an autoimmune disease or an autoimmune disease other than RA.

In another embodiment, the subject does have secondary Sjögren's syndrome or secondary limited cutaneous vasculitis, but not the primary forms thereof.

### VII. Articles of Manufacture

Articles of manufacture containing materials useful for the treatment of the RA or joint damage described above are provided herein. The article of manufacture comprises a container and a label or package insert on or associated with the container. In this aspect, the package insert is on or associated with the container. Suitable containers include, for example, bottles, vials, syringes, etc. The containers may be formed from a variety of materials such as glass or plastic. The container holds or contains the anti-CD20 antibody that is effective for treating the RA or joint damage and may have a sterile access port (for example, the container may be an i.v. solution bag or a vial having a stopper pierceable by a hypodermic injection needle). At least one active agent in the composition is the anti-CD20 antibody. The label or package insert indicates that the composition is used for treating joint damage or RA in a subject eligible for treatment with specific guidance regarding dosing amounts and intervals of antibody and any other medicament being provided.

The article of manufacture may further comprise a second container comprising a pharmaceutically acceptable diluent buffer, such as bacteriostatic water for injection (BWFI), phosphate-buffered saline, Ringer's solution, and dextrose solution. The article of manufacture may further include other materials desirable from a commercial and user standpoint, including other buffers, diluents, filters, needles, and syringes.

The kits and articles of manufacture of the present invention also include information, for example in the form of a package insert or label, indicating that the composition is used for treating RA or joint damage where the patient is not responsive to treatment with rituximab. The insert or label may take any form, such as paper or electronic media, for example, a magnetically recorded medium (e.g., floppy disk) or a CD-ROM. The label or insert may also include other information concerning the pharmaceutical compositions and dosage forms in the kit or article of manufacture.

Generally, such information aids patients and physicians in using the enclosed pharmaceutical compositions and dosage forms effectively and safely. For example, the following information regarding the antibody may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references and patent information.

A specific embodiment of the invention is an article of manufacture comprising, packaged together, a pharmaceutical composition comprising one of the anti-CD20 antibodies herein and a pharmaceutically acceptable carrier and a label stating that the pharmaceutical composition is indicated for treating patients with RA who are not responsive to rituximab treatment. The same label applies to treatment of patients with joint damage.

In a preferred embodiment the article of manufacture herein further comprises a container comprising a second medicament, wherein the antibody is a first medicament, and which article further comprises instructions on the package insert for treating the patient with the second medicament, in an effective amount. The second medicament may be any of those set forth above, with an exemplary second medicament being those set forth above, including an immunosuppressive agent, a corticosteroid, a DMARD, a different antibody against CD20 than the first medicament, an integrin antagonist, a NSAID, a cytokine antagonist, a bisphosphonate, or a combination thereof, more preferably a DMARD, NSAID, cytokine antagonist, integrin antagonist, or immunosuppressive agent. Most preferably, the second medicament is MTX.

In another aspect, the invention provides a method for manufacturing an anti-CD20 antibody herein or a pharmaceutical composition thereof comprising combining in a package the antibody or pharmaceutical composition and a label stating that the anti-CD20 antibody or pharmaceutical composition is indicated for treating patients with RA or joint damage who are not responsive to rituximab.

### VIII. Methods of Advertising

The invention herein also encompasses a method for advertising a pharmaceutically acceptable composition of one of the anti-CD20 antibodies herein comprising promoting, to a target audience, the use of the composition in an effective amount for treating a patient with RA or joint damage who is non-responsive to rituximab.

Advertising is generally paid communication through a non-personal medium in which the sponsor is identified and the message is controlled. Advertising for purposes herein includes publicity, public relations, product placement, sponsorship, underwriting, and sales promotion. This term also includes sponsored informational public notices appearing in any of the print communications media designed to appeal to a mass audience to persuade, inform, promote, motivate, or otherwise modify behavior toward a favorable pattern of purchasing, supporting, or approving the invention herein.

The advertising and promotion of the treatment methods herein may be accomplished by any means. Examples of advertising media used to deliver these messages include television, radio, movies, magazines, newspapers, the internet, and billboards, including commercials, which are messages appearing in the broadcast media. Advertisements also include those on the seats of grocery carts, on the walls of an airport walkway, and on the sides of buses, or heard in telephone hold messages or in-store PA systems, or anywhere a visual or audible communication can be placed. More specific examples of promotion or advertising means include television, radio, movies, the internet such as webcasts and webinars, interactive computer networks intended to reach simultaneous users, fixed or electronic billboards and other public signs, posters, traditional or electronic literature such as magazines and newspapers, other media outlets, presentations or individual contacts by, e.g., e-mail, phone, instant message, postal, courier, mass, or carrier mail, in-person visits, etc.

The type of advertising used will depend on many factors, for example, on the nature of the target audience to be reached, e.g., hospitals, insurance companies, clinics, doctors, nurses, and patients, as well as cost considerations and the relevant jurisdictional laws and regulations governing advertising of medicaments and diagnostics. The advertising may be individualized or customized based on user characterizations defined by service interaction and/or other data such as user demographics and geographical location.

Many alternative experimental methods known in the art may be successfully substituted for those specifically described herein in the practice of this invention, as. for example. described in manuals, textbooks and websites available in the areas of technology relevant to this invention (e.g., Using Antibodies, A Laboratory Manual, Harlow, E. and Lane, D., eds. (Cold Spring Harbor Laboratory Press, New York, 1999); Roe et. al., DNA Isolation and Sequencing (Essential Techniques Series) (John Wiley & Sons, 1996); Methods in Enzymology: Chimeric Genes and Proteins, Abelson et al., eds. (Academic Press, 2000); Molecular Cloning: a Laboratory Manual, 3rd Edition, by Sambrook and MacCallum, (Cold Spring Harbor Laboratory Press, New York, 2001); Current Protocols in Molecular Biology, Ausubel et. al., eds. (John Wiley & Sons, 1987) and periodic updates; PCR: The Polymerase Chain Reaction, (Mullis et al., ed., 1994); Current Protocols in Protein Science, Coligan, ed. (John Wiley & Sons, 2003); and Methods in Enzymology: Guide to Protein Purification, Vol. 182, Deutscher, ed. (Academic Press, Inc., 1990)).

Further details of the invention are illustrated by the following non-limiting Examples. The disclosures of all citations in the specification are expressly incorporated herein by reference.

### Example 1

### Treatment of Rituximab-Refractory RA

A patient with active RA who has an inadequate response to rituximab is treated with another anti-CD20 antibody, wherein this anti-CD20 antibody is ofatumumab, veltuzumab, the immunopharmaceutical with SEQ ID NO:11 (i.e., TRU-015), a CD20-binding antibody (i.e., AME 33, AME 133, or AME 133v), or GA101.

Candidates for therapy according to this example include those who have experienced an inadequate response to previous or current treatment with rituximab because of toxicity or inadequate efficacy. In this previous or current treatment, rituximab is given to the patient in one or two doses of 1 g each; if two doses, they are given two weeks apart.

Patients may have swollen joint count (SJC)≧8 (66 joint count), and tender joint count (TJC)≧8 (68 joint count) at screening and randomization; either C-reactive protein (CRP)≧1.5 mg/dl (15 mg/L) or erythrocyte sedimentation rate (ESR)≧28 mm/h; and/or radiographic evidence of at least one joint with definite erosion attributable to RA as determined by the central reading site (any joint of the hands, wrists or feet can be considered with the exception of the distal interphalangeal joint (DIP) joints of the hands).

Patients are treated with the ofatumumab, veltuzumab, TRU-015, AME 33, AME 133, AME 133v, or GA101 using a dosage regimen selected from: 100 mg×1-4, 200 mg×1-4, 300×1-4, 400 mg×1-4, 500 mg×1-4, 600 mg×1-4, 700 mg×1-4, 800 mg×1-4, 900 mg×1-4, 1000 mg×1-4, or 2000 mg×1-4. Multidoses are given one or two weeks apart within a month's time.

Patients may also receive concomitant MTX (10-25 mg/week per oral (p.o.) or parenteral), together with a corticosteroid regimen consisting of methylprednisolone 100 mg i.v. 30 minutes prior to infusions of the anti-CD20 antibody and prednisone 60 mg p.o. on Days 2-7, 30 mg p.o. Days 8-14, returning to baseline dose by Day 16. Patients may also receive folate (5 mg/week) given as either a single dose or as divided daily doses. Patients optionally continue to receive any background corticosteroid (<10 mg/d prednisone or equivalent) throughout the treatment period. The patients may also receive rituximab (for example, 1 g×1-2 (every other week if 2 doses) of rituximab) with the anti-CD20 antibody.

The primary endpoint may be the proportion of patients with an ACR20 response at Week 24 using a Cochran-Mantel-Haenszel (CMH) test for comparing group differences, adjusted for RF and region.

Potential secondary endpoints include:
1. Proportion of patients with ACR50 and 70 responses at Week 24. These may be analyzed as specified for the primary endpoint.
2. Change in Disease Activity Score (DAS) from screening to Week 24. These may be assessed using an ANOVA model with baseline DAS, RF, and treatment as terms in the model.
3. Categorical DAS responders (EULAR response) at Week 24. These may be assessed using a CMH test adjusted for RF.
4. Changes from screening in ACR core set (SJC, TJC, patient's and physician's global assessments, Health Assessment Questionnaire (HAQ), pain, CRP, and ESR). Descriptive statistics may be reported for these parameters.
5. Changes from screening in SF-36. Descriptive statistics may be reported for the eight domain scores and the mental and physical component scores. In addition, the mental and physical component scores may be further categorized and analyzed.
6. Change in modified Sharp radiographic total score, erosion score, and joint space narrowing score. These may be analyzed using continuous or categorical methodology, as appropriate.

Exploratory endpoints and analysis:

ACR(20/50/70 and ACR n) and change in DAS responses over Weeks 8, 12, 16, 20, 24 and beyond will be assessed using a binary or continuous repeated measures model, as appropriate. Exploratory radiographic analyses including proportion of patients with no erosive progression may be assessed at weeks 24 and beyond.

Further exploratory endpoints (e.g., complete clinical response and disease-free period) will be analyzed descriptively as part of the extended observation period. Changes from Screen in FACIT-F fatigue will be analyzed with descriptive statistics.

RA patients with an inadequate response to rituximab therapy as described above who are treated with the ofatumumab, veltuzumab, TRU-015, AME 33, AME 133, AME 133v, or GA101 as detailed above are expected to exhibit a beneficial clinical response according to any one or more of the endpoints noted herein.

What is claimed is:

1. A method of treating a rheumatoid arthritis (RA) patient who is not responsive to rituximab comprising administering an anti-CD20 antibody to the patient in an amount effective to treat the RA, wherein the anti-CD20 antibody is (1) ofatumumab comprising the variable light amino acid sequence in SEQ ID NO:2 and the variable heavy amino acid sequence in SEQ ID NO:4 or in SEQ ID NO:5; (2) veltuzumab comprising the variable light amino acid sequence in SEQ ID NO:7 and the variable heavy amino acid sequence in SEQ ID NO:8 or in SEQ ID NO:9; (3) an immunopharmaceutical comprising SEQ ID NO:11; (4) a CD20-binding antibody comprising the variable light amino acid sequence in SEQ ID NO:13 and the variable heavy amino acid sequence in SEQ ID NO:15, or comprising the variable light amino acid sequence in SEQ ID NO:17 and the variable heavy amino acid sequence in SEQ ID NO:18, or comprising SEQ ID NO:19; or (5) a humanized type II anti-CD20 IgG1 antibody with bisected afucosylated carbohydrates in its Fc region and comprising the variable light amino acid sequence in SEQ ID NO:21 and the variable heavy amino acid sequence in SEQ ID NO:23.

2. The method of claim 1 wherein the anti-CD20 antibody is ofatumumab.

3. The method of claim 2 wherein the ofatumumab comprises the variable heavy amino acid sequence in SEQ ID NO:4.

4. The method of claim 2 wherein the ofatumumab comprises the variable heavy amino acid sequence in SEQ ID NO:5.

5. The method of claim 1 wherein the anti-CD20 antibody is veltuzumab.

6. The method of claim 5 wherein the veltuzumab comprises the variable heavy amino acid sequence in SEQ ID NO:8.

7. The method of claim 5 wherein the veltuzumab comprises the variable heavy amino acid sequence in SEQ ID NO:9.

8. The method of claim 1 wherein the anti-CD20 antibody is the immunopharmaceutical.

9. The method of claim 1 wherein the anti-CD20 antibody is the CD20-binding antibody.

10. The method of claim 9 wherein the CD20-binding antibody comprises the variable light amino acid sequence in SEQ ID NO:13 and the variable heavy amino acid sequence in SEQ ID NO:15.

11. The method of claim 9 wherein the CD20-binding antibody comprises the variable light amino acid sequence in SEQ ID NO:17 and the variable heavy amino acid sequence in SEQ ID NO:18.

12. The method of claim 9 wherein the CD20-binding antibody comprises SEQ ID NO:19.

13. The method of claim 1 wherein the anti-CD20 antibody is the humanized type II anti-CD20 IgG1 antibody.

14. The method of claim 1 wherein the anti-CD20 antibody is not conjugated with a cytotoxic agent.

15. The method of claim 1 wherein the anti-CD20 antibody is administered intravenously.

16. The method of claim 1 wherein the anti-CD20 antibody is administered subcutaneously.

17. The method of claim 1 wherein the effective amount of the anti-CD20 antibody results in a clinical improvement as determined by assessing the number of tender or swollen joints, conducting a global clinical assessment of the patient, assessing erythrocyte sedimentation rate, assessing the amount of C-reactive protein level, or using composite measures of disease activity.

18. The method of claim 1 wherein the anti-CD20 antibody is administered in a dose of between about 50 and 4000 mg.

19. The method of claim 18 wherein the dose is between about 75 and 3000 mg.

20. The method of claim 18 wherein the dose is between about 100 and 2000 mg.

21. The method of claim 18 wherein the dose is between about 100 and 1000 mg.

22. The method of claim 18 wherein the dose is between about 150 and 1000 mg.

23. The method of claim 18 wherein the dose is between about 200 and 1000 mg.

24. The method of claim 18 wherein the dose is about 200, 300, 400, 500, 600, 700, 800, 900, 1000 mg, or 2000 mg.

25. The method of claim 1 wherein the anti-CD20 antibody is administered at a frequency of one to four doses within a period of about one month.

26. The method of claim 1 wherein the anti-CD20 antibody is administered in two to three doses.

27. The method of claim 1 wherein the anti-CD20 antibody is administered within a period of about 2 to 3 weeks.

28. The method of claim 1 further comprising administering an effective amount of one or more second medicaments with the anti-CD20 antibody, wherein the anti-CD20 antibody is a first medicament.

29. The method of claim 28 wherein the second medicament is more than one medicament.

30. The method of claim 27 wherein the second medicament is an immunosuppressive agent, a disease-modifying anti-rheumatic drug (DMARD), a different antibody against CD20 than the first medicament, a pain-control agent, an integrin antagonist, a non-steroidal anti-inflammatory drug (NSAID), a cytokine antagonist, a bisphosphonate, or a combination thereof.

31. The method of claim 30 wherein the second medicament is a DMARD.

32. The method of claim 31 wherein the DMARD is selected from the group consisting of auranofin, chloroquine, D-penicillamine, injectable gold, oral gold, hydroxychloroquine, sulfasalazine, myocrisin and methotrexate.

33. The method of claim 30 wherein the second medicament is a NSAID.

34. The method of claim 33 wherein the NSAID is selected from the group consisting of: fenbufen, naprosyn, diclofenac, etodolac, indomethacin, aspirin, and ibuprofen.

35. The method of claim 30 wherein the immunosuppressive agent is selected from the group consisting of etanercept, infliximab, adalimumab, leflunomide, anakinra, azathioprine, and cyclophosphamide.

36. The method of claim 30 wherein the second medicament is selected from the group consisting of anti-a4, etanercept, infliximab, etanercept, adalimumab, kinaret, efalizumab, osteoprotegerin (OPG), anti-receptor activator of NFκB ligand (anti-RANKL), anti-receptor activator of NFκB-Fc (RANK-Fc), pamidronate, alendronate, actonel, zolendronate, clodronate, methotrexate, azulfidine, hydroxychloroquine, doxycycline, leflunomide, sulfasalazine (SSZ), prednisolone, rituximab, a 2H7 antibody, interleukin-1 receptor antagonist, prednisone, and methylprednisolone.

37. The method of claim 30 wherein the second medicament is selected from the group consisting of infliximab, methotrexate (MTX), a combination of infliximab with MTX, etanercept, a corticosteroid, cyclosporin A, azathioprine, auranofin, hydroxychloroquine (HCQ), a combination of prednisolone with MTX and SSZ, a combination of MTX with SSZ and HCQ, a combination of cyclophosphamide with azathioprine and HCQ, and a combination of adalimumab with MTX.

38. The method of claim 37 wherein the corticosteroid is prednisone, prednisolone, methylprednisolone, hydrocortisone, or dexamethasone.

39. The method of claim 37 wherein the second medicament is MTX.

40. The method of claim 39 wherein the MTX is administered perorally or parenterally.

41. The method of claim 1 wherein the patient has exhibited an inadequate response to one or more anti-tumor necrosis factor-alpha inhibitors.

42. The method of claim 41 wherein the antibody administered as a single dose or as two doses, with each dose being between about 200 mg and 1000 mg.

43. The method of claim 42 wherein the antibody is administered at a dose of about 200 mg×2, about 300 mg×2, about 500 mg×2, about 700 mg×2, or about 1000 mg×2 on days 1 and 15 at the start of the treatment.

44. The method of claim 1 wherein the RA is early RA or incipient RA.

45. The method of claim 1 further comprising re-treating the patient by administering an effective amount of the antibody to the patient.

46. The method of claim 45 wherein the re-treatment is commenced at least about 24 weeks after the first administration of the antibody.

47. The method of claim 45 wherein a further re-treatment is commenced.

48. The method of claim 47 wherein the further re-treatment is commenced at least about 24 weeks after the second administration of the anti-CD20 antibody.

49. The method of claim 45 wherein joint damage has been reduced after the re-treatment.

50. The method of claim 45 wherein clinical improvement is observed in the patient before re-treatment.

51. The method of claim 50 wherein the clinical improvement is determined by assessing the number of tender or swollen joints, conducting a global clinical assessment of the patient, assessing erythrocyte sedimentation rate, assessing the amount of C-reactive protein level, or using composite measures of disease activity.

52. A method for treating joint damage in a subject who is not responsive to rituximab comprising administering to the subject an anti-CD20 antibody that is (1) ofatumumab comprising the variable light amino acid sequence in SEQ ID NO:2 and the variable heavy amino acid sequence in SEQ ID NO:4 or in SEQ ID NO:5; (2) veltuzumab comprising the variable light amino acid sequence in SEQ ID NO:7 and the variable heavy amino acid sequence in SEQ ID NO:8 or in SEQ ID NO:9; (3) an immunopharmaceutical comprising SEQ ID NO:11; (4) a CD20-binding antibody comprising the variable light amino acid sequence in SEQ ID NO:13 and the variable heavy amino acid sequence in SEQ ID NO:15, or comprising the variable light amino acid sequence in SEQ ID NO:17 and the variable heavy amino acid sequence in SEQ ID NO:18, or comprising SEQ ID NO:19; or (5) a humanized type II anti-CD20 IgG1 antibody with bisected afucosylated carbohydrates in its Fc region and comprising the variable light amino acid sequence in SEQ ID NO:21 and the variable heavy amino acid sequence in SEQ ID NO:23, wherein the amount of anti-CD20 antibody administered is effective in achieving a reduction in the joint damage.

53. The method of claim 52 wherein radiographic testing is used to determine the extent of joint damage reduction.

54. The method of claim 53 wherein the test is done at least about one month after administering the antibody.

55. The method of claim 54 wherein the test is done at least about two months after administering the antibody.

56. The method of claim 52 wherein the anti-CD20 antibody is ofatumumab.

57. The method of claim 56 wherein the ofatumumab comprises the variable heavy amino acid sequence in SEQ ID NO:4.

58. The method of claim 56 wherein the ofatumumab comprises the variable heavy amino acid sequence in SEQ ID NO:5.

59. The method of claim 52 wherein the anti-CD20 antibody is veltuzumab.

60. The method of claim 59 wherein the veltuzumab comprises the variable heavy amino acid sequence in SEQ ID NO:8.

61. The method of claim 59 wherein the veltuzumab comprises the variable heavy amino acid sequence in SEQ ID NO:9.

62. The method of claim 52 wherein the anti-CD20 antibody is the immunopharmaceutical.

63. The method of claim 52 wherein the anti-CD20 antibody is the CD20-binding antibody.

64. The method of claim 63 wherein the CD20-binding antibody comprises the variable light amino acid sequence in SEQ ID NO:13 and the variable heavy amino acid sequence in SEQ ID NO:15.

65. The method of claim 63 wherein the CD20-binding antibody comprises the variable light amino acid sequence in SEQ ID NO:17 and the variable heavy amino acid sequence in SEQ ID NO:18.

66. The method of claim 63 wherein the CD20-binding antibody comprises SEQ ID NO:19.

67. The method of claim 52 wherein the anti-CD20 antibody is the humanized type II anti-CD20 IgG1 antibody.

68. The method of claim 52 further comprising administering an effective amount of one or more second medicaments with the anti-CD20 antibody, wherein the anti-CD20 antibody is a first medicament.

69. The method of claim 68 wherein the second medicament is more than one medicament.

70. The method of claim 67 wherein the second medicament is an immunosuppressive agent, a disease-modifying anti-rheumatic drug (DMARD), a different antibody against CD20 than the first medicament, an integrin antagonist, a non-steroidal anti-inflammatory drug (NSAID), a cytokine antagonist, a bisphosphonate, or a combination thereof.

71. A method for advertising an anti-CD20 antibody or a pharmaceutically acceptable composition thereof comprising promoting, to a target audience, the use of an anti-CD20 antibody that is (1) ofatumumab comprising the variable light amino acid sequence in SEQ ID NO:2 and the variable heavy amino acid sequence in SEQ ID NO:4 or in SEQ ID NO:5; (2) veltuzumab comprising the variable light amino acid sequence in SEQ ID NO:7 and the variable heavy amino acid sequence in SEQ ID NO:8 or in SEQ ID NO:9; (3) an immunopharmaceutical comprising SEQ ID NO:11; (4) a CD20-binding antibody comprising the variable light amino acid sequence in SEQ ID NO:13 and the variable heavy amino acid sequence in SEQ ID NO:15, or comprising the variable light amino acid sequence in SEQ ID NO:17 and the variable heavy amino acid sequence in SEQ ID NO:18, or comprising SEQ ID NO:19; or (5) a humanized type II anti-CD20 IgG1 antibody with bisected afucosylated carbohydrates in its Fc region and comprising the variable light amino acid sequence in SEQ ID NO:21 and the variable heavy amino acid sequence in SEQ ID NO:23, or a pharmaceutical composition thereof for treating a rheumatoid arthritis patient who is not responsive to rituximab.


SEQUENCE LISTING (42 sequences)

SEQ ID NO: 1 - DNA, 382 bp, Artificial Sequence
  Sequence is synthesized.
atggaagccccagctcagcttctcttcctcctgctactctggctcccagataccaccggagaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggccgatcaccttcggccaagggacacgactggagattaaac

SEQ ID NO: 2 - protein, 127 aa, Artificial sequence
  Sequence is synthesized.
MEAPAQLLFLLLLWLPDTTGEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 3 - DNA, 424 bp, Artificial sequence
  Sequence is synthesized.
atggagttgggactgagctggattttccttttggctattttaaaaggtgtccagtgtgaagtgcagctggtggagtctgggggaggcttggtacagcctggcaggtccctgagactctcctgtgcagcctctggattcacctttaatgattatgccatgcactgggtccggcaagctccagggaagggcctggagtgggtctcaactattagttggaatagtggttccataggctatgcggactctgtgaagggccgattcaccatctccagagacaacgccaagaagtccctgtatctgcaaatgaacagtctgagagctgaggacacggccttgtattactgtgcaaaagatatacagtacggcaactactactacggtatggacgtctggggccaagggaccacggtcaccgtctcctcag

SEQ ID NO: 4 - protein, 141 aa, Artificial sequence
  Sequence is synthesized.
MELGLSWIFLLAILKGVQCEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 5 - protein, 141 aa, Artificial sequence
  Sequence is synthesized.
MFLGLSWIFLLAILKGVQCEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 6 - protein, 19 aa, Artificial sequence
  Sequence is synthesized.
MGWSCIILFLVATATGVHS

SEQ ID NO: 7 - protein, 106 aa, Artificial sequence
  Sequence is synthesized.
DIQLTQSPSSLSASVGDRVTMTCRASSSVSYIHWFQQKPGKAPKPWIYATSNLASGVPVRFSGSGSGTDYTFTISSLQPEDIATYYCQQWTSNPPTFGGGTKLEIK

SEQ ID NO: 8 - protein, 120 aa, Artificial sequence
  Sequence is synthesized.
QVQLQQSGAEVKKPGSSVKVSCKASGYTFTSYNMHWVKQAPGQGLEWIGAIYPGNGDTSYNQKFKGKATLTADESTNTAYMELSSLRSEDTAFYYCARSTYYGGDWYFDVWGQGTTVTVS

SEQ ID NO: 9 - protein, 120 aa, Artificial sequence
  Sequence is synthesized.
QVQLQQSGAEVKKPGSSVKVSCKASGYTFSSYNMHWVRQAPGQGLEWMGAIYPGNGDTSYNQKFKGRATITADESTNTAYMELSSLRSEDTAFYFCARSTYYGGDWYFDVWGQGTTVTVS

SEQ ID NO: 10 - DNA, 1518 bp, Artificial sequence
  Sequence is synthesized.
aagcttgccgccatggattttcaagtgcagattttcagcttcctgctaatcagtgcttcagtcataatgtccagaggacaaattgttctctcccagtctccagcaatcctgtctgcatctccaggggagaaggtcacaatgacttgcagggccagctcaagtgtaagttacatgcactggtaccagcagaagccaggatcctcccccaaaccctggatttatgccccatccaacctggcttctggagtccctgctcgcttcagtggcagtgggtctgggacctcttactctctcacaatcagcagagtggaggctgaagatgctgccacttattactgccagcagtggagttttaacccacccacgttcggtgctgggaccaagctggagctgaaagatggcggtggctcgggcggtggtggatctggaggaggtgggagctctcaggcttatctacagcagtctggggctgagtcggtgaggcctggggcctcagtgaagatgtcctgcaaggcttctggctacacatttaccagttacaatatgcactgggtaaagcagacacctagacagggcctggaatggattggagctatttatccaggaaatggtgatacttcctacaatcagaagttcaagggcaaggccacactgactgtagacaaatcctccagcacagcctacatgcagctcagcagcctgacatctgaagactctgcggtctatttctgtgcaagagtggtgtactatagtaactcttactggtacttcgatgtctggggcacagggaccacggtcaccgtctctgatcaggagcccaaatcttgtgacaaaactcacacatctccaccgtgctcagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgatctaga

SEQ ID NO: 11 - protein, 499 aa, Artificial sequence
  Sequence is synthesized.
MDFQVQIFSFLLISASVIMSRGQIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKDGGGSGGGGSGGGGSSQAYLQQSGAESVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSDQEPKSCDKTHTSPPCSAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - DNA, 318 bp, Artificial sequence
  Sequence is synthesized.
gaaattgtgttgacgcagtctccaggcaccctgtctttgtctccaggggaaagagccaccctctcctgcagggccagctcaagtgtaccgtacatccactggtaccagcagaaacctggccaggctcccaggctcctcatctatgccacatccgctctggcttctggcatcccagacaggttcagtggcagtgggtctgggacagacttcactctcaccatcagcagactggagcctgaagattttgcagtgtattactgtcagcagtggctgagtaacccacccacttttggccaggggaccaagctggagatcaaa

SEQ ID NO: 13 - protein, 106 aa, Artificial sequence
  Sequence is synthesized.
EIVLTQSPGTLSLSPGERATLSCRASSSVPYIHWYQQKPGQAPRLLIYATSALASGIPDRFSGSGSGTDFTLTISRLEPEDFAVYYCQQWLSNPPTFGQGTKLEIK

SEQ ID NO: 14 - DNA, 363 bp, Artificial sequence
  Sequence is synthesized.
gaggtgcagctggtgcagtctggagcagaggtgaaaaagcccggggagtctctgaagatctcctgtaagggttctggccgtacatttaccagttacaatatgcactgggtgcgccagatgcccgggaaaggcctggagtggatgggggctatttatcccttgacgggtgatacttcctacaatcagaagtcgaaactccaggtcaccatctcagccgacaagtccatcagcaccgcctacctgcagtggagcagcctgaaggcctcggacaccgccatgtattactgtgcgagatcgacttacgtgggcggtgactggcagttcgatgtctggggcaaggggaccacggtcaccgtctcctca

SEQ ID NO: 15 - protein, 121 aa, Artificial sequence
  Sequence is synthesized.
EVQLVQSGAEVKKPGESLKISCKGSGRTFTSYNMHWVRQMPGKGLEWMGAIYPLTGDTSYNQKSKLQVTISADKSISTAYLQWSSLKASDTAMYYCARSTYVGGDWQFDVWGKGTTVTVSS

SEQ ID NO: 16 - DNA, 642 bp, Artificial sequence
  Sequence is synthesized.
gaaattgtgttgacgcagtctccaggcaccctgtctttgtctccaggggaaagagccaccctctcctgcagggccagctcaagtgtaccgtacatccactggtaccagcagaaacctggccaggctcccaggctcctcatctatgccacatccgctctggcttctggcatcccagacaggttcagtggcagtgggtctgggacagacttcactctcaccatcagcagactggagcctgaagattttgcagtgtattactgtcagcagtggctgagtaacccacccacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 17 - protein, 213 aa, Artificial sequence
  Sequence is synthesized.
EIVLTQSPGTLSLSPGERATLSCRASSSVPYIHWYQQKPGQAPRLLIYATSALASGIPDRFSGSGSGTDFTLTISRLEPEDFAVYYCQQWLSNPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 121 aa, Artificial sequence; identical to SEQ ID NO: 15
  Sequence is synthesized.

SEQ ID NO: 19 - protein, 765 aa, Artificial sequence [sequence omitted]

SEQ ID NO: 20 - DNA, 345 bp, Artificial sequence
  Sequence is synthesized.
gatatcgtgatgacccagactccactctccctgcccgtcacccctggagagcccgccagcattagctgcaggtctagcaagagcctcttgcacagcaatggcatcacttatttgtattggtacctgcaaaagccagggcagtctccacagctcctgatttatcaaatgtccaaccttgtctctggcgtccctgaccggttctccggatccgggtcaggcactgatttcacactgaaaatcagcagggtggaggctgaggatgttggagtttattactgcgctcagaatctagaacttccttacaccttcggcggagggaccaaggtggagatcaaacgtacggtg

SEQ ID NO: 21 - protein, 115 aa, Artificial sequence
  Sequence is synthesized.
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTV

SEQ ID NO: 22 - DNA, 357 bp, Artificial sequence
  Sequence is synthesized.
caggtgcaattggtgcagtctggcgctgaagttaagaagcctgggagttcagtgaaggtctcctgcaaggcttccggatacgccttcagctattcttggatgaactgggtgcggcaggcccctggacaagggctcgagtggatgggacggatctttcccggcgatggggatactgactacaatgggaaattcaagggcagagtcacaattaccgccgacaaatccactagcacagcctatatggagctgagcagcctgagatctgaggacacggccgtgtattactgtgcaagaaatgtctttgatggttactggcttgtttactggggccagggaaccctggtcaccgtctcctca

SEQ ID NO: 23 - protein, 119 aa, Artificial sequence
  Sequence is synthesized.
QVQLVQSGAEVKKPGSSVKVSCKASGYAFSYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 24 - protein, 107 aa, Artificial sequence
  Sequence is synthesized.
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKR

SEQ ID NO: 25 - protein, 122 aa, Artificial sequence [sequence omitted]

SEQ ID NO: 26 - protein, 107 aa, Artificial sequence
  Sequence is synthesized.
DIQMTQSPSSLSASVGDRVTITCRASSSVSYLHWYQQKPGKAPKPLIYAPSNLASGVPSRFSGSGSGTDFTLTISSLQPEDFATYYCQQWAFNPPTFGQGTKVEIKR

SEQ ID NO: 27 - protein, 122 aa, Artificial sequence
  Sequence is synthesized.
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSS

SEQ ID NO: 28 - protein, 122 aa, Artificial sequence
  Sequence is synthesized.
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSYRYWYFDVWGQGTLVTVSS

SEQ ID NO: 29 - protein, 213 aa, Artificial sequence
  Sequence is synthesized.
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 451 aa, Artificial sequence
  Sequence is synthesized.
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 451 aa, Artificial sequence
  Sequence is synthesized.
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - protein, 213 aa, Artificial sequence
  Sequence is synthesized.
DIQMTQSPSSLSASVGDRVTITCRASSSVSYLHWYQQKPGKAPKPLIYAPSNLASGVPSRFSGSGSGTDFTLTISSLQPEDFATYYCQQWAFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 451 aa, Artificial sequence
  Sequence is sythesized.
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - protein, 451 aa, Artificial sequence
  Sequence is synthesized.
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCAVSNKALPAPIEATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 35 - protein, 451 aa, Artificial sequence
  Sequence is synthesized.
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - protein, 451 aa, Artificial sequence
  Sequence is synthesized.
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 37 - protein, 451 aa, Artificial sequence
  Sequence is synthesized.
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSYRYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 38 - protein, 451 aa, Artificial sequence
  Sequence is synthesized.
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 39 - protein, 106 aa, Artificial sequence [sequence omitted]

SEQ ID NO: 40 - protein, 121 aa, Artificial sequence
  Sequence is synthesized.
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVS

SEQ ID NO: 41 - protein, 213 aa, Artificial sequence; identical to SEQ ID NO: 17
  Sequence is synthesized.

SEQ ID NO: 42 - protein, 451 aa, Artificial sequence
  Sequence is sythesized.
EVQLVQSGAEVKKPGESLKISCKGSGRTFTSYNMHWVRQMPGKGLEWMGAIYPLTGDTSYNQKSKLQVTISADKSISTAYLQWSSLKASDTAMYYCARSTYVGGDWQFDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK